(19) United States
(12) Patent Application Publication
(10) Pub. No.: US 2024/0219400 A1
(43) Pub. Date: Jul. 4, 2024

(54) PEPTIDE DECORATED NANOPARTICLES FOR ENRICHMENT OF SPECIFIC PROTEIN SUBSETS

(71) Applicant: Seer, Inc., Redwood City, CA (US)

(72) Inventors: Asim Siddiqui, San Francisco, CA (US); Daniel Hornburg, Foster City, CA (US); Craig Stolarczyk, San Mateo, CA (US); Yina Li, Mountain View, CA (US); Hongwei Xia, Fremont, CA (US)

(21) Appl. No.: 18/557,753

(22) PCT Filed: Apr. 29, 2022
(86) PCT No.: PCT/US2022/027080

§ 371 Date: Oct. 27, 2023

Related U.S. Application Data
(60) Provisional application No. 63/181,765, filed on Apr. 29, 2021.

(51) Int. Cl. G01N 33/68; G01N 33/531; G01N 33/543

(52) U.S. Cl. CPC G01N 33/6851; G01N 33/531; G01N 33/54346; G01N 2333/976

(57) ABSTRACT

The present disclosure provides a range of compositions and methods for enriching subsets of complex biological samples. Aspects of the present disclosure provide peptide-functionalized particles comprising affinities for subsets of biomolecules from complex biological samples. The present disclosure further provides methods for utilizing functionalized particles to fractionate and analyze complex biological samples.

### CROSS REFERENCE

This application claims the benefit of U.S. Provisional Patent Application No. 63/181,765 filed Apr. 29, 2021, which is incorporated herein by reference in the entirety for all purposes.

### BACKGROUND

Proteomic analysis is challenged by the wide protein concentration ranges of biological systems. Many organisms comprise proteins over a range of greater than ten orders of magnitude in concentration, rendering many present proteomic methods infeasible for directly detecting low abundance proteins without prior enrichment steps. While a number of enrichment methods enable single-protein collection from samples, such methods are blind to wider proteomic variations and latent consortia-affected complexities of broader expression patterns.

### SUMMARY

Proteomic profiling methods often face tradeoffs between profiling depth and resolution. For many analytical methods (e.g., mass spectrometric protein detection), analyzing proteins over a wide dynamic range can limit the ability to distinguish similar proteins or accurately resolve protein abundances. Thus, enrichment methods that collect hundreds or thousands of proteins may not always be optimal for identifying subtle differences between similar samples. Conversely, highly targeted enrichment methods, such as histological methods, often comprise restrictive binding specificities that miss variations outside of a targeted sub-population. Recognized herein is a need for selective protein enrichment and analysis methods comprising both breadth and tailorable target specificity.

In some aspects, the present disclosure provides a system comprising: a surface; a peptide coupled to the surface, wherein the peptide comprises a binding site; and at least three different proteins bound to the peptide at the binding site.

In some embodiments, the peptide comprises a synthetic sequence.

In some embodiments, the peptide comprises one or more non-natural amino acids.

In some embodiments, the peptide comprises one or more natural amino acids.

In some embodiments, the at least three different proteins comprise at least 4, 5, 6, 7, 8, 9, or 10 different proteins.

In some embodiments, the at least three different proteins comprise at most 3, 4, 5, 6, 7, 8, 9, 10, 100, or 1000 different proteins.

In some embodiments, the at least three different proteins are bound to a single instance of the peptide.

In some embodiments, the at least three different proteins are individually bound to different instances of the peptide.

In some embodiments, the peptide comprises a variant of a given peptide configured to bind to a different biomolecule in the at least three different proteins.

In some embodiments, the binding site comprises at least a portion of the peptide.

In some embodiments, the binding site comprises the entirety of the peptide.

In some embodiments, the peptide comprises a portion of a given binding site of the given peptide.

In some embodiments, the peptide comprises a mutation of the given peptide such that the peptide comprises a modified form of the given binding site of the given peptide.

In some embodiments, the modified form comprises a different geometry than the given binding site of the given peptide.

In some embodiments, the binding site of the modified form comprises different vibrational modes of the binding site of the given peptide, as measured by IR spectra or Raman spectroscopy.

In some embodiments, the peptide comprises less specificity than a given binding site of the given peptide.

In some embodiments, a first free energy of binding between the peptide and a first protein in the at least three different proteins is substantially equal to a second free energy of binding between the peptide and a second protein in the at least three different proteins, as measured in an aqueous solution comprising an ITC buffer.

In some embodiments, the first free energy of binding between the peptide and the first protein in the at least three different proteins is substantially equal to a third free energy of binding between the peptide and a third protein in the at least three different proteins, as measured in the aqueous solution comprising the ITC buffer.

In some embodiments, a first equilibrium constant of binding between the peptide and a first protein in the at least three different proteins is substantially equal to a second equilibrium constant of binding between the peptide and a second protein in the at least three different proteins, as measured in an aqueous solution comprising an ITC buffer.

In some embodiments, the first equilibrium constant of binding between the peptide and the first protein in the at least three different proteins is substantially equal to a third equilibrium constant of binding between the peptide and a third protein in the at least three different proteins, as measured in an aqueous solution comprising an ITC buffer.

In some embodiments, the at least three different proteins comprise at least 4, 5, 6, 7, 8, 9, or 10 different proteins.

In some embodiments, the at least three different proteins comprise at least 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 different proteins.

In some embodiments, the at least three different proteins comprise at most 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 different proteins.

In some embodiments, a first amount of a first protein in the at least three different proteins bound to the peptide and a second amount of a second protein in the at least three different proteins bound to the peptide are within 1 magnitude of each other.

In some embodiments, the first amount of the first protein in the at least three different proteins bound to the peptide and a third amount of a third protein in the at least three different proteins bound to the peptide are within 1 magnitude of each other.

In some embodiments, the first amount and the second amount are within 2, 3, 4, or 5 magnitude of each other.

In some embodiments, the first amount and the third amount are within 2, 3, 4, or 5 magnitude of each other.

In some embodiments, the first amount and the second amount are intensity measured with mass spectrometry.

In some embodiments, the first amount and the third amount are intensity measured with mass spectrometry.

In some embodiments, the first amount and the second amount may be measured when the surface is in contact with water, a buffer, or an aqueous solution comprising a co-solvent.

In some embodiments, the first amount and the second amount may be measured when the surface has a temperature between 15 and 25 degrees Celsius.

In some embodiments, the peptide is coupled to the surface at a density of at least about 1 peptide per 5 nanometers, 1 peptide per 50 nanometers squared, or 1 peptide per 500 nanometers.

In some embodiments, the peptide comprises at least 20 amino acids.

In some embodiments, the peptide comprise a plurality modular units.

In some embodiments, the peptide comprises at least 2, 5, or 10 amino acids.

In some embodiments, the peptide comprises at least 30, 40, 50, 60, 70, 80, 90, or 100 amino acids.

In some embodiments, the peptide comprises at most 30, 40, 50, 60, 70, 80, 90, or 100 amino acids.

In some embodiments, the peptide comprises a substantially linear domain.

In some embodiments, the substantially linear domain may comprise an alpha-helix conformation.

In some embodiments, the surface further comprises a plurality of peptides coupled thereto, wherein each peptide in the plurality of peptides is configured to bind to at least three different proteins.

In some embodiments, a first peptide in the plurality of peptides is configured to bind to a first set of proteins, and a second peptide in the plurality of peptides is configured to bind to a second set of proteins, wherein the first set and the second are different.

In some embodiments, the at least three different proteins comprise the same epitope.

In some embodiments, the at least three different proteins comprise at least two proteoforms expressed at least partially from the same locus of exons.

In some embodiments, the at least three different proteins comprise at least 3, 4, 5, 6, 7, 8, 9, 10, 20, 30, 40, 50, 60, 70, 80, 90, or 100 proteoforms expressed at least partially from the same locus of exons.

In some embodiments, the system further comprises a plurality of biomolecules deposited on the surface, wherein the plurality of biomolecules comprises the at least three different proteins.

In some embodiments, the plurality of biomolecules comprises at least about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 proteins not specifically bound to the peptide.

In some embodiments, the plurality of biomolecules comprises a dynamic range of at least about 5, 6, 7, 8, 9, 10, 11, 12, 13, 14, 15, 16, 17, 18, 19, or 20.

In some embodiments, the surface is provided in a solution with at least about 0.05, 0.1, 0.2, 0.3, 0.4, or 0.5 cm² in surface area of the surface per μL of the solution.

In some embodiments, the surface is provided in the solution with at most about 0.1, 0.2, 0.3, 0.4, 0.5, or 1.0 in surface area of the surface per μL of the solution.

In some embodiments, the surface is provided in a solution with at least about 1, 2, 3, 4, 5, or 10 μg in mass of a substance comprising the surface per μL of the solution.

In some embodiments, the surface is provided in the solution with at most about 2, 3, 4, 5, or 20 μg in mass of the substance comprising the surface per μL of the solution.

In some embodiments, the surface is provided in a solution with at least about 1, 2, 3, 4, 5, or 10 μg in mass of a substance comprising the surface per mg of the solution.

In some embodiments, the surface is provided in the solution with at most about 2, 3, 4, 5, or 20 μg in mass of the substance comprising the surface per mg of the solution.

In some embodiments, the system further comprises a solvent.

In some embodiments, the solvent comprises one or more ions.

In some embodiments, the solvent comprising the one or more ions decreases solubility of nonpolar molecules.

In some embodiments, the solvent comprising the one or more ions increases solubility of nonpolar molecules.

In some embodiments, the one or more ions are selected from the group consisting of: F⁻, SO₄²⁻, HPO₄²⁻, C₂H₃O₂⁻, Cl⁻, Br⁻, NO₃⁻, ClO₃⁻, I⁻, ClO₄⁻, SCN⁻, NH₄⁺, K⁺, Na⁺, Li⁺, Mg²⁺, Ca²⁺, guanidium, or any combination thereof.

In some embodiments, the one or more ions have a Hofmeister effect on one or more biomolecules in the at least three proteins.

In some embodiments, the surface is disposed in a porous material.

In some embodiments, the surface is disposed on a particle.

In some embodiments, the particle comprises a paramagnetic material.

In some embodiments, the particle comprises a core comprising the paramagnetic material.

In some embodiments, the paramagnetic material comprises iron oxide.

In some embodiments, the particle comprises a nanoparticle or a microparticle.

In some embodiments, the particle forms a biomolecule corona comprising a plurality of biomolecules.

In some embodiments, the plurality of biomolecules is adsorbed on the surface.

In some embodiments, the plurality of biomolecules is non-specifically bound to the surface.

In some embodiments, the plurality of biomolecules is captured on the surface such that a ratio of abundance between at least two biomolecules in the plurality of biomolecules in solution is changed for the at least two biomolecules in the plurality of biomolecules captured on the surface.

In some embodiments, the plurality of biomolecules is increased in visibility in a downstream assay.

In some embodiments, the visibility of a biomolecule in the plurality of biomolecules is intensity measured with mass spectrometry.

In some embodiments, the visibility of a biomolecule in the plurality of biomolecules is quantity measured with nucleic acid quantitation.

In some embodiments, the plurality of biomolecules is improved in composition when captured on the surface such that the plurality of biomolecules is improved for identification in a downstream assay.

In some embodiments, the downstream assay is mass spectrometry.

In some embodiments, the downstream assay is nucleic acid sequencing.

In some embodiments, the peptide is covalently coupled to the surface.

In some embodiments, the surface comprises a silica layer comprising the surface.

In some embodiments, the silica layer comprises a linker that covalently couples the peptide to the surface.

In some embodiments, the linker comprises at least one of: a silanization chemistry, a PEGylation chemistry, a maleimide chemistry, a succinimidyl ester chemistry, an isothiocyanate chemistry, a click chemistry, a thiol chemistry, a trans-4-(Maleimidomethyl)cyclohexanecarboxylic Acid-NHS (SMCC) chemistry, a biorthogonal chemistry, or any combination thereof. In some embodiments, the biorthogonal chemistry comprises Staudinger ligation. In some embodiments, the biorthogonal chemistry comprises an azide chemistry. In some embodiments, the biorthogonal chemistry comprises extension by adding amino acids in additive steps and/or click chemistry.

In some embodiments, the linker comprises a density of at least about 1 linker per 1 nanometer squared, 1 linker per 3 nanometers squared, or 1 linker per 30 nanometers squared on the surface.

In some embodiments, the density is at most about 1 linker per nanometer squared, 1 linker per 3 nanometers squared, 1 linker per 30 nanometers squared on the surface, or 1 linker or 300 nanometers squared on the surface.

In some embodiments, the linker comprises a length of at least about 8, 16, 32, 64, 128, 256, 512, 1024 covalent bonds from the surface to the peptide.

In some embodiments, the length is at most about 8, 16, 32, 64, 128, 256, 512, 1024, or 2048 covalent bonds from the surface to the peptide.

In some embodiments, the linker comprises dimensions sufficient for the peptide to extend at least about 2 nm away from the surface.

In some embodiments, the linker comprises dimensions sufficient for the peptide to extend at least about 2, 5, 10, 20, 30, 40, 50, 60, 70, 80, 90, or 100 nm away from the surface.

In some embodiments, the linker comprises dimensions sufficient for the peptide to extend at most about 2, 5, 10, 20, 30, 40, 50, 60, 70, 80, 90, 100, or 200 nm away from the surface.

In some embodiments, the surface comprises a surface zeta potential of about 13-35 mV.

In some embodiments, the surface comprises a surface zeta potential of at least about 0.01 mV in magnitude.

In some embodiments, the surface comprises a surface zeta potential of at least about 0.1 mV in magnitude, wherein the surface comprises a surface zeta potential of at least about 1 mV in magnitude.

In some embodiments, the surface comprises a surface zeta potential of at least about 10 mV in magnitude.

In some embodiments, the surface comprises a surface zeta potential of at least about 100 mV in magnitude.

In some embodiments, the surface comprises a surface zeta potential of at least about 1000 mV in magnitude.

In some embodiments, the surface comprises a hydrophobic surface or a hydrophilic surface.

In some embodiments, the surface comprises an elemental carbon fraction of about 10-43% as measured by XPS.

In some embodiments, the surface comprises an elemental oxygen fraction of about 40-60% as measured by XPS.

In some embodiments, the surface comprises an elemental nitrogen fraction of about 1-4% as measured by XPS.

In some embodiments, the surface comprises an elemental sulfur composition of about 0.5-2% as measured by XPS.

In some embodiments, the surface comprises an elemental silicon composition of about 24-31% as measured by XPS.

In some embodiments, the surface comprises an elemental iron composition of about 0% as measured by XPS.

In some embodiments, the XPS is performed up to a depth of at most about 5 nm.

In some embodiments, the XPS is performed up to a depth of at most about 1 nm.

In some embodiments, the XPS is performed up to a depth of at most about 10 nm.

In some aspects, the present disclosure provides a system comprising an N number of surfaces, wherein an n-th surface in the N number of surfaces comprises: (a) an n-th peptide coupled to the n-th surface; and (b) an n-th plurality of distinct biomolecules non-specifically bound to the n-th peptide, wherein Nis at least 2, and n ranges from 1 to N.

In some embodiments, N is at least 3, 4, 5, 6, 7, 8, 9, or 10.

In some embodiments, N is at most 3, 4, 5, 6, 7, 8, 9, or 10.

In some embodiments, at least one n-th plurality of distinct biomolecules comprises at least 3, 4, 5, 6, 7, 8, 9, or 10 different biomolecules.

In some embodiments, at least one n-th plurality of distinct biomolecules comprises at most 3, 4, 5, 6, 7, 8, 9, or 10 different biomolecules.

In some embodiments, at least 2, 3, 4, 5, 6, 7, 8, 9, or 10 n-th plurality of distinct biomolecules each comprise at least 3, 4, 5, 6, 7, 8, 9, or 10 different biomolecules.

In some embodiments, at least 2, 3, 4, 5, 6, 7, 8, 9, or 10 n-th plurality of distinct biomolecules each comprise at most 3, 4, 5, 6, 7, 8, 9, or 10 different biomolecules.

In some embodiments, a first amount of a first biomolecule in at least one n-th plurality of distinct biomolecules and a second amount of a second biomolecule in the at least one n-th plurality of distinct biomolecules is within 1 magnitude of each other.

In some embodiments, the first amount of the first biomolecule in the at least one n-th plurality of distinct biomolecules and a third amount of a third biomolecule in the at least one n-th plurality of distinct biomolecules is within 1 magnitude of each other.

In some embodiments, the first amount and the second amount are within 2, 3, 4, or 5 magnitude of each other.

In some embodiments, the first amount and the third amount are within 2, 3, 4, or 5 magnitude of each other.

In some embodiments, the first amount and the second amount are intensity measured with mass spectrometry.

In some embodiments, the first amount and the third amount are intensity measured with mass spectrometry.

In some embodiments, the first amount and the second amount are measured with nucleic acid quantitation.

In some embodiments, the first amount and the third amount are measured with nucleic acid quantitation.

In some embodiments, the first amount and the second amount may be measured when the surface is in contact with water, a buffer, or an aqueous solution comprising a co-solvent.

In some embodiments, the first amount and the second amount may be measured when the surface has a temperature between 15 and 25 degrees Celsius.

In some embodiments, the first amount and the third amount may be measured when the surface is in contact with water, a buffer, or an aqueous solution comprising a co-solvent.

In some embodiments, the first amount and the third amount may be measured when the surface has a temperature between 15 and 25 degrees Celsius.

In some embodiments, at least one n-th peptide comprises a variant of a given peptide configured to bind to a different biomolecule in the at least three different proteins.

In some embodiments, the at least one n-th peptide comprises a portion of a given binding site of the given peptide.

In some embodiments, the at least one n-th peptide comprises a mutation of the given peptide such that the at least one n-th peptide comprises a modified form of the given binding site of the given peptide.

In some embodiments, the modified form comprises a different geometry than the given binding site of the given peptide.

In some embodiments, the modified form comprises different vibrational modes, as measured by IR spectra or Raman spectroscopy.

In some embodiments, the at least one n-th peptide comprises less specificity than a given binding site of the given peptide.

In some embodiments, a first free energy of binding between the at least one n-th peptide and a first biomolecule in at least one n-th plurality of distinct biomolecules and a second free energy of binding between the at least one n-th peptide and a second biomolecule in the at least one n-th plurality of distinct biomolecules is within 1 magnitude of each other.

In some embodiments, the first free energy of binding between the at least one n-th peptide and the first biomolecule in at least one n-th plurality of distinct biomolecules and a third free energy of binding between the at least one n-th peptide and a third biomolecule in the at least one n-th plurality of distinct biomolecules is within 1 magnitude of each other.

In some embodiments, the first free energy is within 200% of the second free energy.

In some embodiments, the first free energy is within 200% of the third free energy.

In some embodiments, a first equilibrium constant of binding between the at least one n-th peptide and a first biomolecule in at least one n-th plurality of distinct biomolecules is substantially equal to a second equilibrium constant of binding between the at least one n-th peptide and a second biomolecule in the at least one n-th plurality of distinct biomolecules, as measured in an aqueous solution comprising an ITC buffer.

In some embodiments, the first free energy of binding between the at least one n-th peptide and the first biomolecule in at least one n-th plurality of distinct biomolecules is substantially equal to a third free energy of binding between the at least one n-th peptide and a third biomolecule in the at least one n-th plurality of distinct biomolecules, as measured in an aqueous solution comprising an ITC buffer.

In some embodiments, the first equilibrium constant is within 200% of the second equilibrium constant.

In some embodiments, the second equilibrium constant is within 200% of the third equilibrium constant.

In some embodiments, the at least one n-th peptide is coupled to the surface at a density of at least about 1 peptide per 50 nanometers squared.

In some embodiments, the peptide is coupled to the surface at a density of at most about 1 peptide per 5, 50, or 500 nanometers squared.

In some embodiments, the peptide is coupled to the surface at a density of at least about 1 peptide per 5, 50, or 500 nanometers squared.

In some embodiments, the at least one n-th peptide comprises at least 20 amino acids.

In some embodiments, the at least one n-th peptide comprises at most 40 amino acids.

In some embodiments, the peptide comprise a plurality of modular units.

In some embodiments, the at least one n-th peptide comprises a substantially linear domain.

In some embodiments, at least one n-th surface further comprises a plurality of peptides coupled thereto, wherein each peptide in the plurality of peptides is configured to bind to at least three different proteins.

In some embodiments, the at least three different proteins in the at least one n-th plurality of distinct biomolecules comprise the same epitope.

In some embodiments, the at least three different proteins comprise at least two proteoforms expressed at least partially from the same locus of exons.

In some aspects, the present disclosure provides a method comprising: contacting a biological sample with a surface to bind a plurality of biomolecules to a peptide coupled to the surface, wherein the peptide is configured to bind to at least three different proteins; releasing the plurality of biomolecules or a portion thereof from the surface; and identifying at least the plurality of biomolecules or the portion thereof, wherein the plurality of biomolecules or the portion thereof comprises one or more biomolecules in the at least three different proteins in the biological sample.

In some embodiments, the identifying comprises performing mass spectrometry on the plurality of biomolecules.

In some embodiments, the identifying comprises performing nucleic acid sequencing on the plurality of biomolecules.

In some aspects, the present disclosure provides a method comprising: contacting a surface with a first composition, wherein: the surface comprises a peptide coupled thereto, wherein the peptide is configured to specifically bind to at least three different target biomolecules; and the first composition comprises a plurality of biomolecules, wherein the plurality of biomolecules comprises at least one target biomolecule in the at least three different target biomolecules; such that: the peptide binds to the at least one target biomolecule in the at least three different target biomolecules; and the plurality of biomolecules adsorbs on the surface; contacting the surface with a proteolytic enzyme to release: the at least one target biomolecule; at least a subset of the plurality of biomolecules adsorbed on the surface; and at least a portion of the peptide; thereby producing a second composition; performing mass spectrometry on the second composition to identify: the at least one target biomolecule; the at least the subset of the plurality of biomolecules; and the at least the portion of the peptide; thereby generating a composition measurement for the first composition; and removing, from the composition measurement, one or more signals originating from the at least the portion of the peptide, thereby generating a refined composition measurement for the first composition.

In some aspects, the present disclosure provides a means for some challenges disclosed herein by providing particles with broad and controlled protein enrichment profiles. In some aspects, the present disclosure provides a plurality of particles which (i) collect one or more given subsets of proteins each having a limited number of proteins (e.g., 10-500) from a sample and/or (ii) collect non-overlapping or substantially non-overlapping subsets of higher abundance proteins often generate higher resolution proteomic profiles. In some aspects, the present disclosure provides a range of strategies for maximizing proteomic profiling resolution.

Various aspects of the present disclosure provide a composition comprising a particle having on its surface less than 5 distinct types of binding molecules, said binding molecules having binding specificity for a subset of proteins in a sample, wherein said subset of proteins comprises about 10-500 proteins.

In some embodiments, said particle comprises a single type of binding molecule. In some embodiments, said particle comprises 2-5 distinct types of binding molecules. In some embodiments, said binding molecules comprise a peptide. In some embodiments, said peptide comprises between 7 and 20 amino acids. In some embodiments, said peptide comprises between 7 and 15 amino acids. In some embodiments, said peptide comprises between 8 and 12 amino acids.

In some embodiments, said peptide comprises amino acids selected from the group consisting of alanine, arginine, aspartic acid, glutamic acid, glutamine, glycine, histidine, isoleucine, leucine, lysine, proline, serine, asparagine, threonine, and valine. In some embodiments, said peptide is free of one or more of cysteine, methionine, tryptophan, tyrosine, and phenylalanine. In some embodiments, said peptide comprises a non-proteinogenic amino acid. In some embodiments, said non-proteinogenic amino acid is a chemically modified proteinogenic amino acid. In some embodiments, said chemical modification comprises acylation, alkylation, amidation, deamidation, carbamylation, carbonylation, carboxylation, decarboxylation, citrullination, flavination, glycosylation, halogenation, hydroxylation, nitrosylation, oxidation, phosphorylation, prenylation, racemization, reduction, succinylation, sulfation, or any combination thereof.

In some embodiments, said peptide comprises an isoelectric point (pI) of between 5 and 10. In some embodiments, said particle comprises an isoelectric point of within 0.5 of said peptide. In some embodiments, a conformation of said peptide comprises a dependence on pH, temperature, ionic strength, dielectric constant, viscosity, or any combination thereof.

In some embodiments, said peptide is coupled to said surface of said particle. In some embodiments, said peptide is coupled directly to said surface of said particle. In some embodiments, a C-terminus of said peptide is coupled to said surface of said particle. In some embodiments, an N-terminus of said peptide is coupled to said surface of said particle. In some embodiments, an internal amino acid of said peptide is coupled to said surface of said particle. In some embodiments, said peptide is coupled to a chemical linker coupled to said surface of said particle. In some embodiments, a C-terminus of said peptide is coupled to said chemical linker. In some embodiments, an N-terminus of said peptide is coupled to said chemical linker. In some embodiments, an internal amino acid of said peptide is coupled to said chemical linker. In some embodiments, said N-terminus or said internal amino acid comprises an amide bond to said chemical linker. In some embodiments, said chemical linker comprises a maleimide group.

In some embodiments, said particle comprises 2 to 5 distinct types of peptides. In some embodiments, said 2 to 5 distinct peptides comprise at least two peptides having different lengths. In some embodiments, said 2 to 5 distinct peptides have substantially similar isoelectric points. In some embodiments, said 2 to 5 distinct peptides have at least two different isoelectric points. In some embodiments, said particle comprises specific molar ratios of said 2 to 5 distinct peptides.

In some embodiments, said sample is a biological sample. In some embodiments, said biological sample comprises whole blood, plasma, buffy coat, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, said biological sample comprises plasma or serum. In some embodiments, said biological sample comprises a liquid. In some embodiments, said biological sample comprises greater than 1000 types of proteins. In some embodiments, said biological sample comprises a protein concentration dynamic range of at least 8 orders of magnitude.

In some embodiments, said subset of proteins comprises 60-200 proteins. In some embodiments, said subset of proteins comprises 80-160 proteins. In some embodiments, said subset of proteins comprises 100-140 proteins. In some embodiments, said subset of proteins comprises 10-50 proteins. In some embodiments, said subset of proteins comprises 10-30 proteins. In some embodiments, said subset of proteins comprises a dynamic range of at least 4 orders of magnitude. In some embodiments, said subset of proteins comprises a dynamic range of at least 6 orders of magnitude. In some embodiments, said subset of proteins comprises a high abundance protein. In some embodiments, said high abundance protein comprises at least 0.1% of the protein mass of said sample. In some embodiments, said high abundance protein comprises a concentration of at least 1 micromolar (μM).

In some embodiments, said composition comprises a plurality of particles comprising said particle. In some embodiments, individual particles of said plurality of particles comprise the same binding molecules. In some embodiments, said binding specificity comprises at most 1 micromolar (μM) dissociation constants. In some embodiments, said binding specificity comprises at most 1 nanomolar (nM) dissociation constants. In some embodiments, said binding molecules comprise an average spacing of at least 4 nanometers (nm) on said surface of said particle. In some embodiments, said binding molecules comprise an average spacing of at least 10 nanometers (nm) on said surface of said particle.

Various aspects of the present disclosure provide a method for enriching a subset of proteins in a sample, comprising: contacting said sample with a composition comprising a particle, said particle having on its surface less than 5 distinct types of binding molecules which have binding specificity for said subset of proteins in said sample; and capturing said subset of proteins using said particle, to thereby enrich said subset of proteins, wherein said subset of proteins comprises about 10-500 proteins.

In some embodiments, said contacting comprises adding between 100 picomolar (pM) and 100 nanomolar (nM) of said particle to said sample.

In some embodiments, said capturing comprises incubating said particle in said sample for at least 1 hour. In some embodiments, said capturing comprises incubating said particle in said sample at a temperature of at least 37° C. In some embodiments, said capturing comprises collecting at least 10⁻⁹ mg of said subset of said proteins per square millimeter (mm²) of surface area of said particle. In some embodiments, said enriching comprises narrowing a dynamic range of said subset of proteins.

In some embodiments, said particle comprises a single type of binding molecule. In some embodiments, said particle comprises 2-5 distinct types of binding molecules. In some embodiments, said binding molecules comprise a peptide. In some embodiments, said peptide comprises between 7 and 20 amino acids. In some embodiments, said peptide is free of one or more of cysteine, methionine, tryptophan, tyrosine, and phenylalanine. In some embodiments, said peptide comprises an isoelectric point (pI) of between 5 and 10. In some embodiments, said particle comprises an isoelectric point of within 0.5 of said peptide. In some embodiments, a conformation of said peptide comprises a dependence on pH, temperature, ionic strength, dielectric constant, viscosity, or any combination thereof. In some embodiments, said peptide comprises 2 to 5 distinct types of peptides. In some embodiments, said 2 to 5 distinct peptides comprise at least two different isoelectric points. In some embodiments, said particle comprises specific molar ratios of said 2 to 5 distinct peptides.

In some embodiments, said sample is a biological sample. In some embodiments, said biological sample comprises whole blood, plasma, buffy coat, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, said biological sample comprises plasma or serum. In some embodiments, said biological sample comprises a liquid. In some embodiments, said biological sample comprises greater than 1000 types of proteins. In some embodiments, said biological sample comprises greater than 5000 types of proteins.

In some embodiments, said subset of proteins comprises 60-200 proteins. In some embodiments, said subset of proteins comprises 10-50 proteins. In some embodiments, said subset of proteins comprises a high abundance protein. In some embodiments, said composition comprises a plurality of particles comprising said particle.

Various aspects of the present disclosure provide a composition for enriching proteins from a sample, said composition comprising at least two distinct particle types each comprising surface bound binding molecules having binding specificity for a different set of proteins of said sample, wherein a set of proteins of a first particle type and a second set of proteins of a second particle type have between 10%-85% overlap.

In some embodiments, said first set of proteins and said second set of proteins comprise at most 70% overlap. In some embodiments, said first set of proteins or said second set of proteins has a dynamic range of at least 4 orders of magnitude. In some embodiments, said dynamic range is at most 6 orders of magnitude. In some embodiments, said surface bound binding molecules comprise no more than 5 distinct types of binding molecules.

In some embodiments, said surface bound binding molecules comprise peptides. In some embodiments, said peptides comprise between 7 and 20 amino acids. In some embodiments, said peptides comprise between 7 and 15 amino acids. In some embodiments, said peptides comprise between 8 and 12 amino acids. In some embodiments, said peptides are free of one or more of cysteine, methionine, tryptophan, tyrosine, and phenylalanine. In some embodiments, said peptides comprise a non-proteinogenic amino acid. In some embodiments, said peptides comprise isoelectric points between 5 and 10. In some embodiments, said peptides comprise 2 to 5 types of peptides per particle type of said at least two distinct particle types. In some embodiments, said 2 to 5 distinct peptides comprise substantially similar isoelectric points. In some embodiments, said 2 to 5 distinct peptides comprise at least two different isoelectric points.

In some embodiments, said given sets of proteins each comprise between 5 and 500 proteins. In some embodiments, said given sets of proteins each comprise between 10 and 500 proteins. In some embodiments, said given sets of proteins each comprise between 10 and 200 proteins. In some embodiments, said given sets of proteins each comprise between 10 and 50 proteins. In some embodiments, said given sets of proteins each comprise between 10 and 30 proteins. In some embodiments, dynamic ranges of first set of proteins and said second set of proteins substantially overlap with each other.

Various aspects of the present disclosure provide a method for assaying a sample, comprising: contacting said sample with a composition comprising a first surface-modified particle configured to enrich for a first subset of proteins in said sample, and a second surface-modified particle configured to enrich for a second subset of proteins in said sample; and enriching for said first subset and said second subset of proteins in said sample using said composition, wherein said first subset of proteins and said second subset of proteins each comprises no more than three proteins that have a concentration of greater than or equal to about 10 μg/mL in said sample, and differ from each other in at least one of such proteins.

In some embodiments, said composition comprises between 100 picomolar (pM) and 100 nanomolar (nM) of said first surface-modified particle and said second surface-modified particle. In some embodiments, said enriching comprises incubating said first surface-modified particle and said second surface-modified particle in said sample for at least 1 hour. In some embodiments, said enriching comprises incubating said first surface-modified particle and said second surface-modified particle in said sample at a temperature of at least 37° C. In some embodiments, said enriching comprises collecting at least 10⁻⁹ mg of said subsets of proteins per square millimeter (mm²) of surface area of said first surface-modified particle and/or said second surface-modified particle.

In some embodiments, said first surface-modified particle and said second surface-modified particle each comprise between 1 and 5 distinct types of surface bound binding molecules. In some embodiments, said first surface-modified particle and said second surface-modified particle each comprise between 2 and 5 distinct types of surface bound binding molecules. In some embodiments, said first surface-modified particle and said second surface-modified particle each comprise at most 2 distinct types of surface bound binding molecules. In some embodiments, said first surface-modified particle or said second surface-modified particle comprises exactly 1 type of surface bound binding molecule.

In some embodiments, said first surface-modified particle or said second surface-modified particle comprises surface-bound peptides. In some embodiments, said surface-bound peptides comprise between 7 and 20 amino acids. In some embodiments, said surface-bound peptides are free of one or more of cysteine, methionine, tryptophan, tyrosine, and phenylalanine. In some embodiments, said surface-bound peptides comprise isoelectric points (pI) between 5 and 10. In some embodiments, said first surface-modified particle and said second surface-modified particle comprise different peptides. In some embodiments, said first surface-modified particle and said second surface-modified particle comprise different isoelectric points. In some embodiments, said first surface-modified particle and said second surface-modified particle each comprise between 1 and 5 different peptides.

In some embodiments, said sample is a biological sample. In some embodiments, said biological sample comprises whole blood, plasma, buffy coat, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, said biological sample comprises plasma or serum. In some embodiments, said biological sample comprises greater than 1000 types of proteins.

Various aspects of the present disclosure provide a composition comprising a plurality of particles having surfaces comprising oligopeptides having 7 or more amino acid residues, said oligopeptides having binding specificity for a subset of proteins in a sample, wherein said subset of proteins comprises less than or equal to about 500 proteins.

In some embodiments, said oligopeptides comprise between 7 and 20 amino acid residues. In some embodiments, said oligopeptides comprise between 7 and 15 amino acid residues. In some embodiments, said binding specificity is at most 100 micromolar (μM). In some embodiments, said binding specificity is at least 1 micromolar (μM). In some embodiments, said binding specificity has a pH dependence.

In some embodiments, said plurality of particles comprises at least a first particle having on its surface a first set of oligopeptides and a second particle having on its surface a second set of oligopeptides different from said first set of oligopeptides. In some embodiments, an isoelectric point of said first particle differs from an isoelectric point of said second particle. In some embodiments, said first set and second set of oligopeptides have binding specificity for a first subset of proteins and a second subset of proteins in said sample, respectively, and wherein said first subset of proteins is different from said second subset of proteins. In some embodiments, said first subset of proteins and said second subset of proteins comprise at most 85% overlap. In some embodiments, said subset of proteins comprises a high abundance protein. In some embodiments, said first subset of proteins and said second subset of proteins comprise at most 85% overlap between their high abundance proteins. In some embodiments, said subset of proteins comprises less than or equal to about 200 proteins.

Various aspects of the present disclosure provide method for enriching a subset of proteins in a sample, comprising: contacting said sample with a composition comprising a plurality of particles having surfaces comprising oligopeptides having 7 or more amino acid residues, said oligopeptides having binding specificity for said subset of proteins in said sample; and capturing said subset of proteins using said plurality of particles to thereby enrich said subset of proteins, wherein said subset of proteins comprises less than or equal to about 500 proteins.

In some embodiments, said contacting comprises adding between 100 picomolar (pM) and 100 nanomolar (nM) of said plurality of particles to said sample. In some embodiments, said capturing comprises incubating said plurality of particles in said sample for at least 1 hour. In some embodiments, said capturing comprises incubating said plurality of particles in said sample at a temperature of at least 37° C. In some embodiments, said capturing comprises collecting at least 10⁻⁹ mg of said subset of said proteins per square millimeter (mm²) of surface area of said plurality of particles.

In some embodiments, said oligopeptides comprise between 1 and 5 distinct types of oligopeptides. In some embodiments, said oligopeptides comprise between 7 and 20 amino acids. In some embodiments, said oligopeptides are free of one or more of cysteine, methionine, tryptophan, tyrosine, and phenylalanine.

In some embodiments, said sample is a biological sample. In some embodiments, said biological sample comprises plasma or serum.

In some embodiments, said subset of proteins comprises less than about 200 proteins. In some embodiments, said subset of proteins comprises greater than about 60 proteins. In some embodiments, said subset of proteins comprises about 10 to about 100 proteins. In some embodiments, said subset of proteins comprises about 10 to about 50 proteins.

Various aspects of the present disclosure provide a surface modified particle comprising one or more binding molecules attached to a surface thereof, said one or more binding molecules having a plurality of segments each of which is independently addressable.

In some embodiments, individual segments of said plurality of segments are separated from one another by a linking element. In some embodiments, said linking element is not a peptide-derived linking element. In some embodiments, individual segments of at least a subset of said plurality of segments are interchangeable. In some embodiments, individual segments of said plurality of segments are capable of being removed, modified, or substituted. In some embodiments, said one or more binding molecules comprise polymers, peptides, or a combination thereof. In some embodiments, said polymers are biodegradable. In some embodiments, said peptides comprise oligopeptides having about 7-20 amino acid residues. In some embodiments, said plurality of segments comprise oligopeptides having about 1-6 amino acid residues. In some embodiments, said particle is a nanoparticle having a diameter between 10 nanometers (nm) and 500 nm.

In some aspects, the present disclosure provides a kit comprising a substance comprising a surface and a peptide coupled thereto, wherein the peptide comprises a binding site configured to bind to a set of biomolecules, wherein the set of biomolecules comprises at least three proteins or at least five biomolecules.

In some embodiments, the kit further comprises a second substance comprising a second surface and a second peptide coupled thereto, wherein the second peptide comprises a binding site configured to bind to a second set of biomolecules, wherein the second set of biomolecules is different from the first set of biomolecules.

In some embodiments, the kit further comprises a proteolytic enzyme.

In some embodiments, the proteolytic enzyme is trypsin or lysin.

In some embodiments, the kit further comprises a buffer.

In some embodiments, the buffer comprises a lyse buffer.

In some embodiments, the substance is disposed in a chamber comprising a first cap, and the second substance is disposed in a chamber comprising a second cap, wherein the first cap and the second cap are different in color.

In some embodiments, the substance is disposed in a chamber comprising a first barcode, and the second substance is disposed in a chamber comprising a second barcode, wherein the first barcode and the second barcode are different.

In some embodiments, the kit further comprises a container for containing the substance, wherein the container comprises a barcode.

In some aspects, the present disclosure provides a method comprising: (a) contacting a biological sample with a composition of the present disclosure to capture a plurality of biomolecules to the composition; (b) releasing the plurality of biomolecules or a portion thereof from the composition; (c) identifying at least the plurality of biomolecules or the portion thereof, wherein the plurality of biomolecules or the portion thereof comprises one or more biomolecules in the at least three different proteins in the biological sample.

In some embodiments, the identifying comprises performing mass spectrometry or nucleic acid sequencing on the plurality of biomolecules or the portion thereof released in (b).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a computer system that is programmed or otherwise configured to implement a method of the present disclosure, in accordance with some embodiments.

FIG. 2 provides an example workflow for collecting biomolecules from a biological sample onto particles, in accordance with some embodiments.

FIG. 3 provides an example workflow for a particle-based assay for analyzing biomolecules from a biological sample, in accordance with some embodiments.

FIG. 4 provides an example workflow for assaying biomolecules from a biological sample with magnetic particles, in accordance with some embodiments.

FIG. 5 illustrates the number of proteins collected on and subsequently identified by mass spectrometry following collection on particle panels comprising from 1 to 12 particles, in accordance with some embodiments.

FIG. 6 provides an example of a method for coupling a peptide to a particle, in accordance with some embodiments.

FIG. 7 provides examples of linkers for coupling a binding molecule and a particle, in accordance with some embodiments.

FIG. 8A provides a diagram of a modular unit which may be used for peptide synthesis, in accordance with some embodiments.

FIG. 8B provides an example of a binding molecule comprising modular units and configured for intermolecular or intramolecular exchange, in accordance with some embodiments.

FIG. 9 provides examples of linear and cyclic peptides which may be coupled to a particle surface, in accordance with some embodiments.

FIG. 10 provides examples of flexible and rigid linkers consistent with the present disclosure, in accordance with some embodiments.

FIG. 11A shows a schematic of a peptide functionalized surface binding with a target, in accordance with some embodiments.

FIG. 11B depicts a cartoon of a free energy surface, in accordance with some embodiments.

FIG. 11C depicts a cartoon of a free energy surface, in accordance with some embodiments.

FIG. 11D shows a schematic of a peptide functionalized surface binding with multiple targets, in accordance with some embodiments.

FIG. 12A shows a schematic of a peptide functionalized surface capturing a plurality of biomolecules, in accordance with some embodiments.

FIG. 12B shows a schematic of a peptide functionalized surface capturing a plurality of biomolecules, in accordance with some embodiments.

FIG. 12C shows a schematic of a peptide functionalized surface capturing a plurality of biomolecules, in accordance with some embodiments.

FIG. 13 schematically illustrates a peptide design pipeline, in accordance with some embodiments.

FIG. 14A shows a surface, in accordance with some embodiments. A surface may be functionalized at one or more regions for capturing biomolecules.

FIG. 14B shows a surface, in accordance with some embodiments. A surface may comprise one or more wells or depressions for capturing biomolecules. For example, a functionalized surface may be disposed in a 96 well plate or a 384 well plate.

FIG. 14C shows a surface, in accordance with some embodiments. A surface may be disposed on one or more particles. In some embodiments, the one or more particles may be disposed in one or more wells or depressions.

FIG. 14D shows a surface, in accordance with some embodiments. A surface may be disposed on a plurality particles packed in a channel or a porous material disposed in a channel.

FIG. 14E shows a surface, in accordance with some embodiments. A surface may be disposed on an inner surface of a channel.

FIGS. 14F-14I show surfaces, in accordance with some embodiments. A surface may comprise 1, 2, 3, 4 or any number of distinct surface regions. In some embodiments, a surface may be disposed on a particle. In some embodiments, a particle may be a porous particle.

FIG. 15A shows a chemical structure of a peptide functionalized surface, in accordance with some embodiments.

FIG. 15B shows a chemical structure of a linker, in accordance with some embodiments.

FIG. 16A shows an ethanolic silane route for synthesis, in accordance with some embodiments.

FIG. 16B shows a silica-based semi two-step route for synthesis, in accordance with some embodiments.

FIG. 16C shows a chemical structure of thiol-functionalized particles, in accordance with some embodiments.

FIG. 16D shows carboxylate and amine functionalized particles, in accordance with some embodiments. Carboxylates and amines can enable coupling using Hexafluorophosphate Azabenzotriazole Tetramethyl Uronium (HATU) reagent or 1-Ethyl-3-(3-dimethylaminopropyl)carbodiimide (EDC) reagent.

FIG. 16E shows a synthesis route using a succinate reagent, in accordance with some embodiments.

FIG. 17 shows the number of protein groups identified using particles of the present disclosure, in accordance with some embodiments.

FIG. 18 shows click chemistry synthesis route for functionalizing peptides to surfaces, in accordance with some embodiments.

FIGS. 19A-19P show peptide chemical structures for functionalizing on surfaces, in accordance with some embodiments.

FIG. 20A shows statistics of protein groups detected with S-437 vs S-348, in accordance with some embodiments.

FIGS. 20B-20D show identified protein groups detected with S-437, with both S-437 and S-348, and with S-348, respectively, in accordance with some embodiments.

FIGS. 20E-20G show intensity of protein groups detected with S-437, with both S-437 and S-348, and with S-348, respectively, in accordance with some embodiments.

FIGS. 20H-J show the protein group intensity CV detected with S-437, with both S-437 and S-348, and with S-348, respectively, in accordance with some embodiments.

FIGS. 20K-20M show the number of peptides associated with protein groups detected with S-437, with both S-437 and S-348, and with S-348, respectively, in accordance with some embodiments.

FIG. 20N depicts mapping to UniProt functional keywords of consistent protein groups with S-437, with both S-437 and S-348, and with S-348, respectively, in accordance with some embodiments.

FIGS. 200-20Q show physicochemical characteristics of consistent protein groups detected with S-437, with both S-437 and S-348, and with S-348, respectively, in accordance with some embodiments.

FIG. 21A shows statistics of protein groups detected with S-439 vs S-348, in accordance with some embodiments.

FIGS. 21B-21D show identified protein groups detected with S-439, with both S-439 and S-348, and with S-348, respectively, in accordance with some embodiments.

FIGS. 21E-21G show intensity of protein groups detected with S-439, with both S-439 and S-348, and with S-348, respectively, in accordance with some embodiments.

FIGS. 21H-J show the protein group intensity CV detected with S-439, with both S-439 and S-348, and with S-348, respectively, in accordance with some embodiments.

FIGS. 21K-21M show the number of peptides associated with protein groups detected with S-439, with both S-437 and S-348, and with S-348, respectively, in accordance with some embodiments.

FIG. 21N depicts mapping to UniProt functional keywords of consistent protein groups with S-439, with both S-439 and S-348, and with S-348, respectively, in accordance with some embodiments.

FIGS. 21O-21Q show physicochemical characteristics of consistent protein groups detected with S-439, with both S-439 and S-348, and with S-348, respectively, in accordance with some embodiments.

FIG. 22A shows statistics of protein groups detected with S-348 vs a 5-particle panel, in accordance with some embodiments.

FIGS. 22B-22D show identified protein groups detected with S-348, with both S-348 and a 5-particle panel, and with a 5-particle panel, respectively, in accordance with some embodiments.

FIGS. 22E-22G show intensity of protein groups detected with S-348, with both S-348 and a 5-particle panel, and with a 5-particle panel, respectively, in accordance with some embodiments.

FIGS. 22H-J show the protein group intensity CV detected with S-348, with both S-348 and a 5-particle panel, and with a 5-particle panel, respectively, in accordance with some embodiments.

FIGS. 22K-22M show the number of peptides associated with protein groups detected with S-348, with both S-348 and a 5-particle panel, and with a 5-particle panel, respectively, in accordance with some embodiments.

FIG. 22N depicts mapping to UniProt functional keywords of consistent protein groups with S-348, with both S-348 and a 5-particle panel, and with a 5-particle panel, respectively, in accordance with some embodiments.

FIGS. 220-22Q show physicochemical characteristics of consistent protein groups detected with S-348, with both S-348 and a 5-particle panel, and with a 5-particle panel, respectively, in accordance with some embodiments.

FIG. 23 shows a kit, in accordance with some embodiments.

### DETAILED DESCRIPTION

Biological samples are often complex mixtures comprising vast arrays of biomolecules with disparate properties. In some aspects, the present disclosure provides a range of methods for fractionating, collecting, enriching, and depleting biomolecules from complex biological samples, thereby enabling deep analysis, profiling, and biomolecule detection.

Aspects of the present disclosure provide compositions, systems, and methods for collecting biomolecules on surfaces, porous materials, particles, surface panels and particle panels of multiple distinct surface or particle types, which enrich proteins from a sample. In some embodiments, biomolecules may be collected onto surfaces. In some embodiments, they may form distinct biomolecule coronas on the surfaces of the distinct particle types. In some embodiments, the particle panels disclosed herein can be used in methods of corona analysis to detect thousands of proteins across a wide dynamic range in the span of hours.

Some proteins exhibit high selectivity and high specificity towards binding a target ligand (e.g., another protein). Various biomolecule assays are designed to leverage this principle, for example, in immunoassays where antigen-antibody reactions are disposed on an array to detect the present of antigens in a sample. These methods can have high selectivity and specificity, although they may be intolerable to small or modest variations in the chemical structures of a target protein. This presents a challenge, since specific antibodies may need to be designed for each new target that one seeks to detect the presence of in a sample, even if the new target may have structural or chemical similarities to a target with a known binding partner.

In living cells, various proteoforms for a given protein group may exist. Variations in the amino acid sequence may be introduced, for example, with mutations in the genetic code of an individual. In some embodiments, variations in amino acid sequence may also be introduced with splicing variations. Further, a protein may undergo experience one or more post-translation modifications (e.g., phosphorylation, a misfold, or chaperone assisted folding). Proteoforms, in some cases, may have some chemical, structural, and/or functional characteristics that are preserved across one another. For example, some proteoforms may comprise a same epitope for binding.

In some aspects, the present disclosure provides systems, compositions, and methods for targeting numerous target biomolecules using a peptide. In some embodiments, a peptide of the present disclosure may comprise a chemical structure that is based at least partially on a chemical structure of a predetermined peptide that is known to target a specific binding target. The chemical structure of a peptide of the present disclosure may comprise mutations, chemical modifications, truncations, and other modifications based on the predetermined peptide. Various modifications are contemplated and described in further detail elsewhere in the present disclosure.

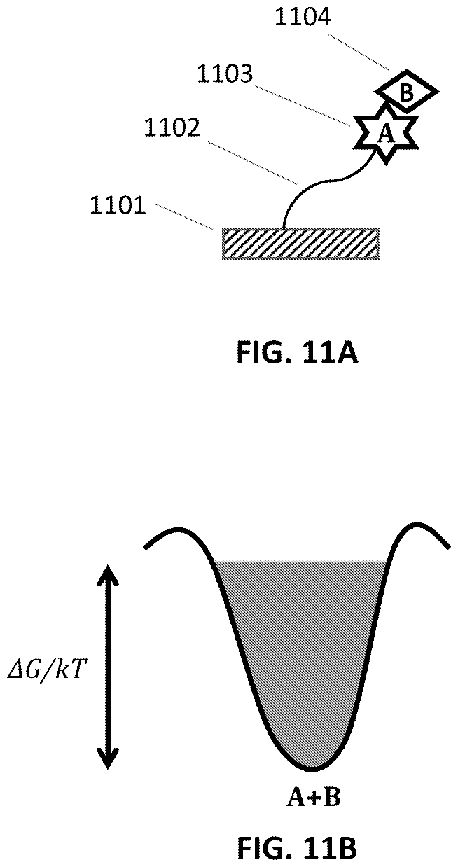

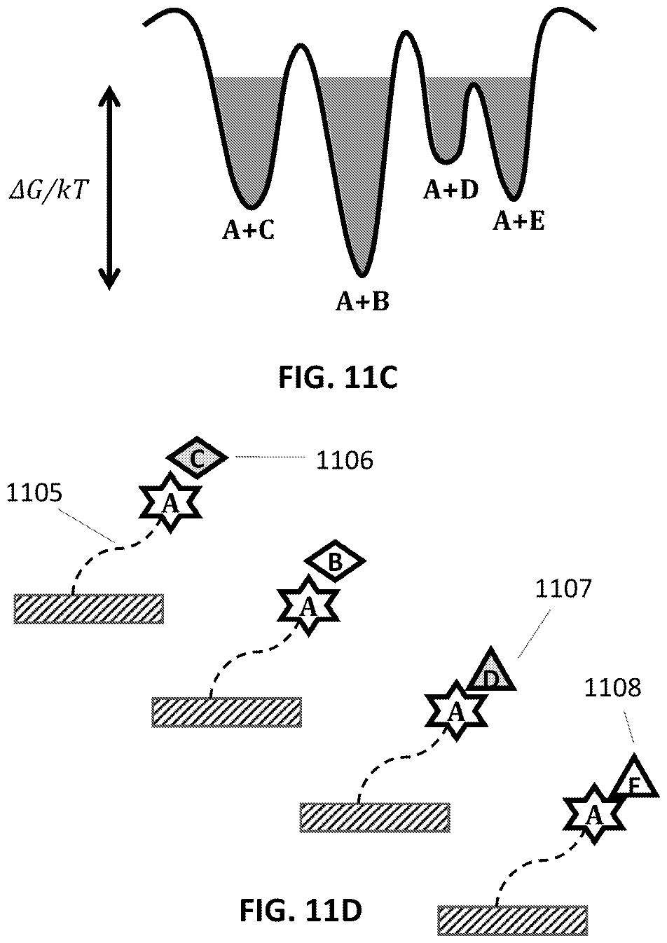

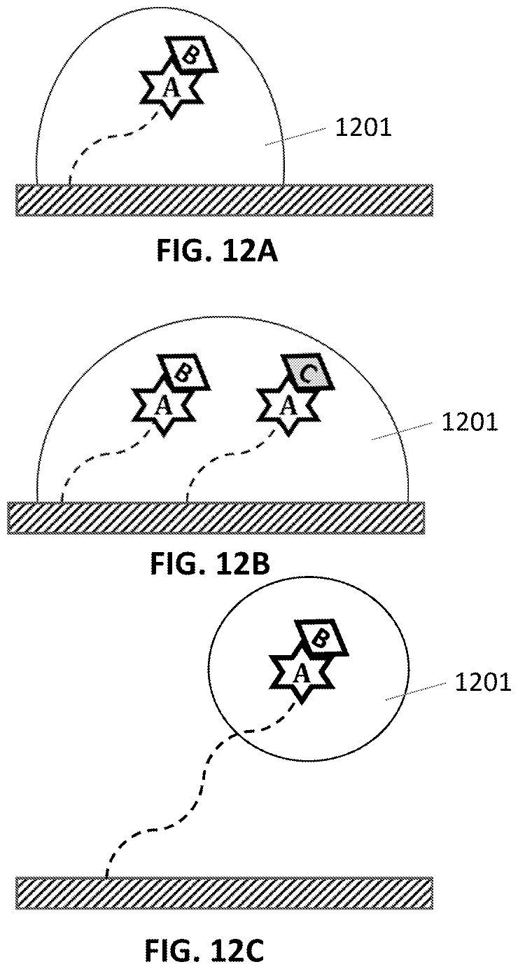

Modifications to a predetermined peptide structure may configure the peptide of the present disclosure to target a plurality of targets. For example, FIG. 11A graphically illustrates a predetermined peptide (1103) functionalized on a surface (1101) via a linker (1102) that is configured to bind to a single target (1104). FIG. 11B depicts a cartoon of a free energy surface for binding the predetermined peptide (1103) and the target (1104). FIG. 11A and FIG. 11B illustrate that a predetermined peptide may have high specificity and high selectivity for one binding targets. In some embodiments, the predetermined peptide may exhibit no significant binding with other targets.

A modification to the chemical structure of the predetermined peptide may lead to a new chemical structure for the peptide that allows binding of numerous targets. FIG. 11C depicts a cartoon of a free energy surface for the peptide that is configured to bind to at least four targets (B, C, D, and E), in accordance with some embodiments. Without being bound to a particular theory, certain modifications in the chemical structure of the predetermined peptide may cause one or more small or moderate changes in the structure of a binding site (for binding targets). In some embodiments, the one or more changes may be chemical in nature, e.g., changes in hydrophilicity, hydrophobicity, ionicity, acidity, basicity, hydrogen-bonding, and/or ability to form covalent bonds. In some embodiments, the one or more changes may be steric in nature, e.g., increased or reduced accessibility of the binding site. In some embodiments, the one or more changes may be entropic in nature, e.g., increased flexibility of the binding site (which may be inferred through analysis of vibrational modes associated with the binding site, and/or free energy surface calculations). Without being bound to a particular theory, these changes may cause changes in the free energy of binding between a peptide and a target biomolecule, and may cause changes in equilibrium constants between a first state comprising a free peptide and a free target biomolecule versus a second state comprising a peptide and a bound target biomolecule. Without being bound to a particular theory, it is contemplated that various physical and chemical changes may alter the binding specificity and/or selectivity of a peptide such that the peptide may be imparted with the ability to bind to multiple targets.

FIG. 11D shows a cartoon depicting a peptide that is configured to bind to a plurality of binding targets, in accordance with some embodiments. In some embodiments, the binding targets may share chemical or structural similarities in at least a portion of their chemical structures, such that they have an affinity towards binding with the peptide. In some embodiments, the binding targets may comprise one or more epitopes in common. In some embodiments, the binding targets may be proteoforms of one another.

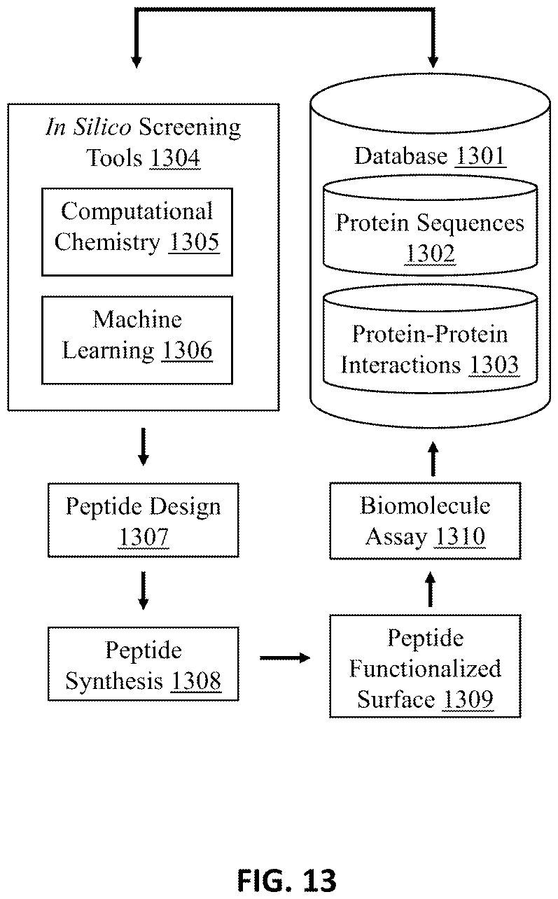

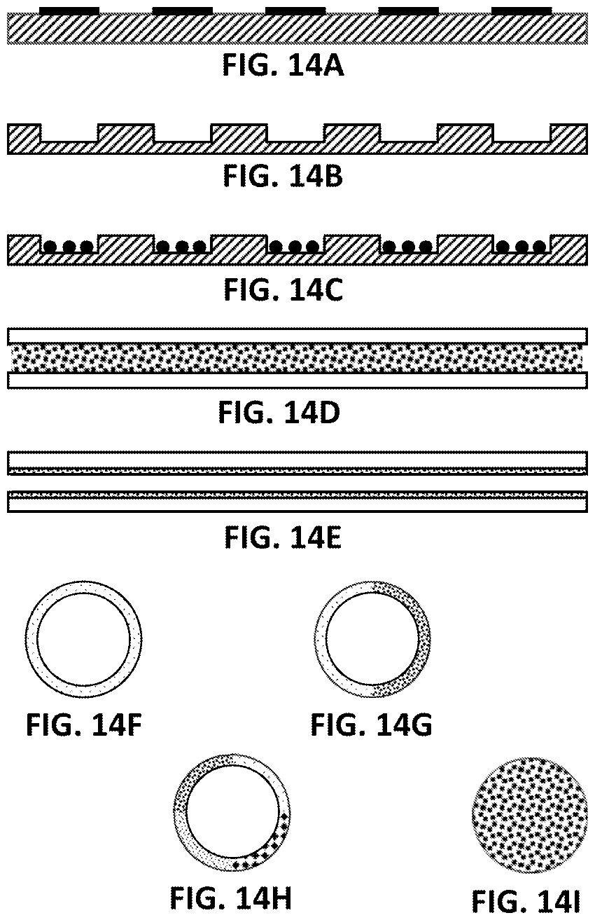

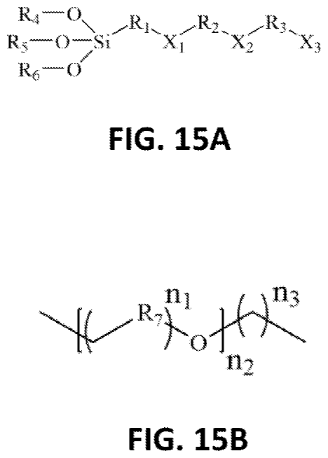

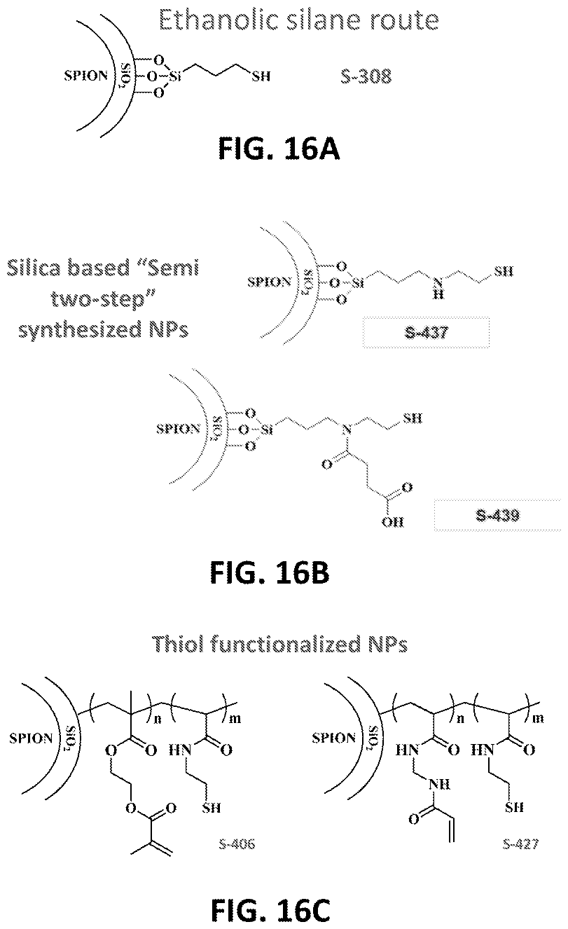

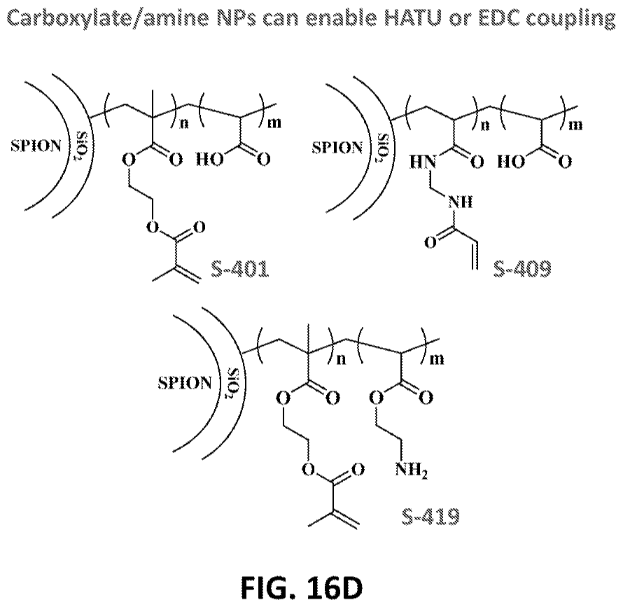

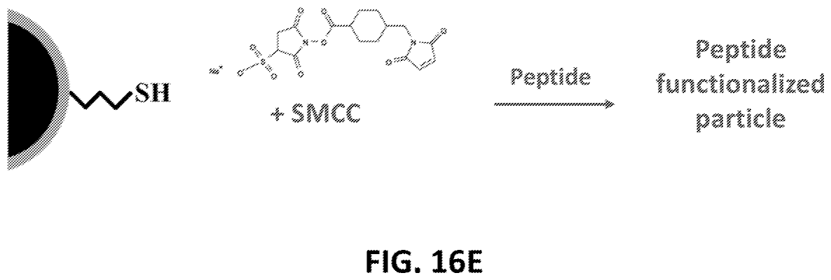

FIG. 13 schematically illustrates a design process for a peptide, in accordance with some embodiments. Designing the peptide may be based at least partially on a database of peptides (1301), in silico screening (1304), experimental synthesis and experiments (1307-1310), or any combination thereof. The peptide may be based at least partially on a chemical structure of a predetermined peptide in a database. In some embodiments, the database may comprise an identifier of a predetermined peptide. In some embodiments, the database may comprise an amino acid sequence of a predetermined peptide (1302). In some embodiments, the database may comprise a 3D structure of a predetermined peptide. In some embodiments, the database may comprise a 3D structure of a predetermined peptide with a bound target. In some embodiments, the database may comprise a list of one or more biomolecules (e.g., proteins) that interact or are expected to interact with the predetermined peptide (1303). In some embodiments, the database may comprise one or more classification labels for the predetermined peptide. In some embodiments, the classification labels may be a label for grouping some peptides by 3D structure, locus or loci of gene expression, involvement in a biochemical pathway, associated with a disease, or any combination thereof.

In some embodiments, an in silico screening methodology or a tool may be applied to design the peptide. In some embodiments, an in silico screening tool may comprise electronic structure calculations, at various levels of theory (e.g., Hartree-Fock, DFT, or coupled-cluster). In some embodiments, an in silico screening tool may comprise molecular dynamics or Monte Carlo simulations (at various levels of detail from atomistic to coarse-grained simulations). In some embodiments, an in silico screening tool may comprise machine learning algorithms. In some embodiments, an in silico screening tool may be used to obtain 3D structures of a peptide. In some embodiments, an in silico screening tool may be used to perform docking simulations between a peptide and one or more targets. In some embodiments, an in silico screening tool may be used to obtain physical quantities associated with binding between a peptide and one or more targets (e.g., free energy or equilibrium constants).

Aspects of the present disclosure provide methods for utilizing and analyzing peptides. In some embodiments, “peptide” may refers to a molecule comprising at least two amino acid residues linked by peptide (e.g., amide) bonds. Non-limiting examples of a peptide include amino acid dimers, trimers, oligomers, or polymers. In some embodiments, a peptide comprises a protein. A peptide may be linear or branched. A peptide may comprise a natural amino acid. A natural amino acid may be a ‘proteinogenic amino acid’, which, as used herein, may refer to any one of the 22 known amino acids utilized for translation by natural organisms, namely alanine, arginine, asparagine, aspartic acid, cystine, glutamic acid, glutamine, glycine, histidine, isoleucine, leucine, lysine, methionine, phenylalanine, proline, serine, threonine, tryptophan, tyrosine, valine, pyrrolysine, and selenocystine. A natural amino acid may be a post-translationally modified amino acid, nonlimiting examples of which include acylated amino acids, alkylated amino acids, prenylated amino acids, nitrosylated amino acids, flavinated amino acids, formylated amino acids, amidated amino acids, deamidated amino acids, halogenated amino acids, carboxylated amino acids, decarboxylated amino acids, glycosylated amino acids, phosphorylated amino acids, sulfurylated amino acids, cyclized amino acids, carbamylated amino acids, carbonylated amino acids, or biotinylated amino acids. A peptide may comprise an isomeric variant of a naturally occurring amino acid, such as an α-carbon enantiomer, also known as a D-amino acid. A peptide may comprise a non-natural (e.g., synthetically derived) amino acid. A non-natural amino acid may comprise a non-natural side chain, such as a perfluorinated aryl or alkyl moiety. A non-natural amino acid may comprise a non-natural backbone structure, for example a silicon in place of the α-carbon or the amine disposed on a β-carbon. A peptide may also comprise non-amino acid units, such as 4-hydroxybutanoic, in place of amino acid residues.

In some aspects, the present disclosure provides a system comprising for assaying biomolecules from a biological sample. In some embodiments, the system comprises a surface. In some embodiments, the system comprises a peptide coupled to the surface. In some embodiments, the peptide comprises a binding site. In some embodiments, at least three different biomolecules are bound to the peptide at the binding site. In some cases, the at least three different biomolecules comprise at least three different proteins. In some embodiments, the at least three different biomolecules comprise at least five different biomolecules. In some embodiments, the at least three different biomolecules are bound to a single instance of the peptide. In some embodiments, the at least three different biomolecules are individually bound to different instances of the peptide. In some embodiments, different biomolecules or proteins may be unique biomolecules or proteins. In some embodiments, the binding site is any portion or entirety of the peptide that participates in binding of a target biomolecule. In some embodiments, the binding site participates in binding of a target biomolecule by contact. In some embodiments, the peptide comprises a variant of a given peptide configured to bind to a different biomolecule in the at least three different proteins. In some embodiments, the peptide comprises a portion of a given binding site of the given peptide.

In some embodiments, the peptide comprises a mutation of the given peptide such that the peptide comprises a modified form of the given binding site of the given peptide. In some embodiments, the modified form comprises a different geometry than the given binding site of the given peptide. In some embodiments, modified form comprises different vibrational modes, as measured by IR spectra or Raman spectroscopy. In some embodiments, the peptide comprises less specificity than a given binding site of the given peptide. In some embodiments, a first free energy of binding between the peptide and a first biomolecule in the at least three different biomolecules is substantially equal to a second free energy of binding between the peptide and a second biomolecule in the at least three different biomolecules, as measured in an aqueous solution comprising an Isothermal Titration calorimetry (ITC) buffer. In some cases, free energy may be measured in water. In some cases, free energy may be measured in a bicarbonate buffer system comprising a physiological pH. In some cases, free energy may be calculated in silico. In some embodiments, ITC buffer can maintain the solubility and stability of the biomolecules. In some embodiments, ITC buffer include salts, cofactors, and additives that are necessary for binding. In some embodiments, ITC buffer has a low enthalpy of ionization. In some embodiments, concentration of ITC buffer is high enough to compensate for any pH effects during titration. In some embodiments, the first free energy of binding between the peptide and the first biomolecule in the at least three different biomolecules is substantially equal to a third free energy of binding between the peptide and a third biomolecule in the at least three different biomolecules, as measured in the aqueous solution comprising the ITC buffer. In some embodiments, a first equilibrium constant of binding between the peptide and a first biomolecule in the at least three different biomolecules is substantially equal to a second equilibrium constant of binding between the peptide and a second biomolecule in the at least three different biomolecules, as measured in an aqueous solution comprising an ITC buffer. In some embodiments, the first equilibrium constant of binding between the peptide and the first biomolecule in the at least three different biomolecules is substantially equal to a third equilibrium constant of binding between the peptide and a third biomolecule in the at least three different biomolecules, as measured in an aqueous solution comprising an ITC buffer.

In some embodiments, the at least three different biomolecules comprise at least 4, 5, 6, 7, 8, 9, or 10 different biomolecules. In some embodiments, the at least three different biomolecules comprise at least 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 different biomolecules. In some embodiments, the at least three different biomolecules comprise at most 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 different biomolecules. In some embodiments, a first amount of a first biomolecule in the at least three different biomolecules bound to the peptide and a second amount of a second biomolecule in the at least three different biomolecules bound to the peptide are within 1 magnitude of each other. In some embodiments, the first amount of the first biomolecule in the at least three different biomolecules bound to the peptide and a third amount of a third biomolecule in the at least three different biomolecules bound to the peptide are within 1 magnitude of each other.

In some embodiments, the peptide is coupled to the surface at a density of at least about 1 peptide per 1 nanometers squared, at least about 1 peptide per 2 nanometers squared, at least about 1 peptide per 3 nanometers squared, at least about 1 peptide per 5 nanometers squared, 1 peptide per 10 nanometers squared, 1 peptide per 20 nanometers squared, 1 peptide per 30 nanometers squared, 1 peptide per 40 nanometers squared, 1 peptide per 50 nanometers squared, 1 peptide per 60 nanometers squared, 1 peptide per 70 nanometers squared, 1 peptide per 80 nanometers squared, 1 peptide per 90 nanometers squared, 1 peptide per 100 nanometers squared, 1 peptide per 150 nanometers squared, 1 peptide per 200 nanometers squared, 1 peptide per 250 nanometers squared, 1 peptide per 300 nanometers squared, 1 peptide per 350 nanometers squared, 1 peptide per 400 nanometers squared, 1 peptide per 450 nanometers squared, or 1 peptide per 500 nanometers squared.

In some embodiments, the peptide is coupled to the surface at a density of at most about 1 peptide per 1 nanometers squared, at most about 1 peptide per 2 nanometers squared, at most about 1 peptide per 3 nanometers squared, at most about 1 peptide per 5 nanometers squared, 1 peptide per 10 nanometers squared, 1 peptide per 20 nanometers squared, 1 peptide per 30 nanometers squared, 1 peptide per 40 nanometers squared, 1 peptide per 50 nanometers squared, 1 peptide per 60 nanometers squared, 1 peptide per 70 nanometers squared, 1 peptide per 80 nanometers squared, 1 peptide per 90 nanometers squared, 1 peptide per 100 nanometers squared, 1 peptide per 150 nanometers squared, 1 peptide per 200 nanometers squared, 1 peptide per 250 nanometers squared, 1 peptide per 300 nanometers squared, 1 peptide per 350 nanometers squared, 1 peptide per 400 nanometers squared, 1 peptide per 450 nanometers squared, or 1 peptide per 500 nanometers squared.

In some embodiments, the peptide comprises at least 5 amino acids, at least 10 amino acids, at least 15 amino acids, at least 20 amino acids, at least 25 amino acids, at least 30 amino acids, at least 35 amino acids, or at least 40 amino acids. In some embodiments, the peptide comprises 5-40 amino acids, 10-35 amino acids, 15-30 amino acids, 20-25 amino acids, or 35-40 amino acids. In some embodiments, the peptide comprises at most 5 amino acids, at most 10 amino acids, at most 15 amino acids, at most 20 amino acids, at most 25 amino acids, at most 30 amino acids, at most 35 amino acids, or at most 40 amino acids.

In some embodiments, the peptide comprises a substantially linear domain. In some embodiments, the peptide comprises a non-linear domain. In some embodiments, a linear domain may comprise an alpha helix. In some embodiments, the non-linear domain may comprise an unstructured domain. In some embodiments, the peptide comprises a circular domain. In some embodiments, the surface further comprises a plurality of peptides coupled thereto, wherein each peptide in the plurality of peptides is configured to bind to at least three different biomolecules. In some embodiments, the at least three different biomolecules comprise the same epitope. In some embodiments, the at least three different biomolecules comprise at least two proteoforms expressed at least partially from the same locus of exons. In some embodiments, the system further comprises a plurality of biomolecules deposited on the surface, wherein the plurality of biomolecules comprises the at least three different biomolecules. In some embodiments, the plurality of biomolecules comprises at least about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 biomolecules not specifically bound to the peptide. In some embodiments, the plurality of biomolecules comprises a dynamic range of at least about 5, 6, 7, 8, 9, 10, 11, 12, 13, 14, 15, 16, 17, 18, 19, or 20.

In some embodiments, the surface is provided in a solution with at least about 0.05, 0.1, 0.2, 0.3, 0.4, or 0.5 cm² in surface area of the surface per μL of the solution. In some embodiments, the surface is provided in the solution with at most about 0.1, 0.2, 0.3, 0.4, 0.5, or 1.0 in surface area of the surface per μL of the solution. In some embodiments, the surface is provided in a solution with at least about 1, 2, 3, 4, 5, or 10 μg in mass of a substance comprising the surface per μL of the solution. In some embodiments, the surface is provided in the solution with at most about 2, 3, 4, 5, or 20 μg in mass of the substance comprising the surface per μL of the solution. In some embodiments, the surface is provided in a solution with at least about 1, 2, 3, 4, 5, or 10 μg in mass of a substance comprising the surface per mg of the solution. In some embodiments, the surface is provided in the solution with at most about 2, 3, 4, 5, or 20 μg in mass of the substance comprising the surface per mg of the solution.

In some embodiments, the particle comprises a paramagnetic material. In some embodiments, the particle comprises a core comprising the paramagnetic material. In some embodiments, the paramagnetic material comprises iron oxide. In some embodiments, the particle comprises a nanoparticle or a microparticle. In some embodiments, the particle forms a biomolecule corona comprising a plurality of biomolecules. In some embodiments, the plurality of biomolecules is adsorbed on the surface. In some embodiments, the plurality of biomolecules is non-specifically bound to the surface. In some embodiments, the plurality of biomolecules is captured on the surface such that a ratio of abundance between at least two biomolecules in the plurality of biomolecules in solution is changed for the at least two biomolecules in the plurality of biomolecules captured on the surface.

In some embodiments, the plurality of biomolecules is increased in visibility in a downstream assay. In some embodiments, the downstream assay is mass spectrometry. In some embodiments, the visibility of a biomolecule the plurality of biomolecules is increased in mass spectrometry intensity. In some embodiments, the downstream assay is nucleic acid sequencing. In some embodiments, the visibility of a biomolecule in the plurality of biomolecules is increased in quantity measured with nucleic acid quantitation. In some embodiments, the peptide is covalently coupled to the surface.

In some embodiments, the surface comprises a silica layer comprising the surface. In some embodiments, the silica layer comprises a linker that covalently couples the peptide to the surface. In some embodiments, the linker comprises at least one of: a silanization chemistry, a PEGylation chemistry, a maleimide chemistry, a succinimidyl ester chemistry, an isothiocyanate chemistry, a click chemistry, a thiol chemistry, SMCC (ADCs linker, Trans-4-(Maleimidomethyl) cyclohexanecarboxylic Acid-NHS), and a biorthogonal chemistry. In some embodiments, a biorthogonal chemistry may comprise a functionality that does not react with one or more functional groups in the peptide.

In some embodiments, the linker comprises a density of at least about 1 linker per 1 nanometer squared, 1 linker per 2 nanometer squared, 1 linker per 3 nanometers squared, 1 linker per 4 nanometer squared, 1 linker per 5 nanometer squared, 1 linker per 6 nanometers squared, 1 linker per 7 nanometer squared, 1 linker per 8 nanometer squared, 1 linker per 9 nanometers squared, 1 linker per 10 nanometer squared, 1 linker per 12 nanometer squared, 1 linker per 14 nanometers squared, 1 linker per 16 nanometer squared, 1 linker per 18 nanometer squared, 1 linker per 20 nanometers squared, 1 linker per 22 nanometer squared, 1 linker per 24 nanometer squared, 1 linker per 26 nanometers squared, 1 linker per 28 nanometers squared, or 1 linker per 30 nanometers squared on the surface. In some embodiments, the linker comprises a length of at least about 8, 16, 32, 64, 128, 256, 512, 1024 covalent bonds from the surface to the peptide. In some embodiments, the linker comprises dimensions sufficient for the peptide to extend at least about 1 nm, 2 nm, 3 nm, 4 nm, or 5 nm away from the surface. In some embodiments, the linker comprises dimensions sufficient for the peptide to extend at 1-5 nm, 2-4 nm, or 3-5 nm away from the surface. In some embodiments, the linker comprises dimensions sufficient for the peptide to extend at most about 1 nm, 2 nm, 3 nm, 4 nm, or 5 nm away from the surface.

In some embodiments, the surface comprises a surface zeta potential of about 13-35 mV in magnitude. In some embodiments, the surface comprises a surface zeta potential of at least about 0.01 mV in magnitude. In some embodiments, the surface comprises a surface zeta potential of at least about 0.1 mV in magnitude. In some embodiments, the surface comprises a surface zeta potential of at least about 1 mV in magnitude. In some embodiments, the surface comprises a surface zeta potential of at least about 10 mV in magnitude. In some embodiments, the surface comprises a surface zeta potential of at least about 100 mV in magnitude. In some embodiments, the surface comprises a surface zeta potential of at least about 1000 mV in magnitude. In some embodiments, the surface comprises a surface zeta potential of at least about 0.01 mV, 0.05 mV, 0.1 mV, 0.5 mV, 1 mV, 5 mV, 10 mV, 50 mV, 100 mV, 500 mV, or 1000 mV in magnitude. In some embodiments, the surface comprises a surface zeta potential between 0.01 mV and 1000 mV, between 0.05 mV and 500 mV, between 0.1 mV and 100 mV, between 0.5 mV and 50 mV, between 1 mV and 10 mV, between 5 mV and 100 mV, or between 500 mV and 1000 mV in magnitude. In some embodiments, the surface zeta potential is positive. In some embodiments, the surface zeta potential is negative.

In some embodiments, the surface comprises a hydrophobic surface. In some embodiments, the surface comprises a hydrophilic surface. In some embodiments, the surface comprises an elemental carbon fraction of about 10-43% as measured by XPS. In some embodiments, the surface may comprise an elemental carbon fraction of at least about 1, 2, 3, 4, 5, 6, 7, 8, 9, 10, 20, 30, 40, 50, 60, 70, 80, or 90%. In some embodiments, the surface may comprise an elemental carbon fraction of at most about 1, 2, 3, 4, 5, 6, 7, 8, 9, 10, 20, 30, 40, 50, 60, 70, 80, or 90%. In some embodiments, the surface comprises an elemental oxygen fraction of about 40-60% as measured by XPS. In some embodiments, the surface may comprise an elemental oxygen fraction of at least about 1, 2, 3, 4, 5, 6, 7, 8, 9, 10, 20, 30, 40, 50, 60, 70, 80, or 90%. In some embodiments, the surface may comprise an elemental oxygen fraction of at most about 1, 2, 3, 4, 5, 6, 7, 8, 9, 10, 20, 30, 40, 50, 60, 70, 80, or 90%. In some embodiments, the surface comprises an elemental nitrogen fraction of about 1-4% as measured by XPS. In some embodiments, the surface may comprise an elemental nitrogen fraction of at least about 0.1, 0.5, 1, 2, 3, 4, 5, 6, 7, 8, 9, 10, or 20%. In some embodiments, the surface may comprise an elemental nitrogen fraction of at most about 0.1, 0.5, 1, 2, 3, 4, 5, 6, 7, 8, 9, 10, or 20%. In some embodiments, the surface comprises an elemental sulfur composition of about 0.5-2% as measured by XPS. In some embodiments, the surface may comprise an elemental sulfur fraction of at least about 0.1, 0.5, 1, 2, 3, 4, 5, 6, 7, 8, 9, or 10%. In some embodiments, the surface may comprise an elemental sulfur fraction of at most about 0.1, 0.5, 1, 2, 3, 4, 5, 6, 7, 8, 9, or 10%. In some embodiments, the surface comprises an elemental silicon composition of about 24-31% as measured by XPS. In some embodiments, the surface may comprise an elemental silicon fraction of at least about 1, 2, 3, 4, 5, 6, 7, 8, 9, 10, 20, 30, 40, 50, 60, or 70%. In some embodiments, the surface may comprise an elemental oxygen fraction of at most about 1, 2, 3, 4, 5, 6, 7, 8, 9, 10, 20, 30, 40, 50, 60, or 70%. In some embodiments, the surface comprises an elemental iron composition of about 0% as measured by XPS. In some embodiments, the surface may comprise an elemental iron fraction of at least about 0.1, 0.5, 1, 2, 3, 4, 5, 6, 7, 8, 9, or 10%. In some embodiments, the surface may comprise an elemental iron fraction of at most about 0.1, 0.5, 1, 2, 3, 4, 5, 6, 7, 8, 9, or 10%. In some embodiments, the XPS is performed up to a depth of at most about 1, 2, 3, 4, 5, 6, 7, 8, 9, 10, 11, 12, 13, 14, or 15 nm. In some embodiments, the XPS is performed up to a depth of at most about 1-10 nm, 2-9 nm, 3-8 nm, 4-7 nm, 5-6 nm or 9-10 nm.

In some aspects, the present disclosure provides a system comprising an N number of surfaces. In some embodiments, an n-th surface in the N number of surfaces comprises an n-th peptide coupled to the n-th surface. In some embodiments, an n-th surface in the N number of surfaces comprises an n-th plurality of distinct biomolecules non-specifically bound to the n-th peptide, wherein N is at least 2, and n ranges from 1 to N. In some embodiments, N is at least 3, 4, 5, 6, 7, 8, 9, or 10. In some embodiments, at least one n-th plurality of distinct biomolecules comprises at least 3, 4, 5, 6, 7, 8, 9, or 10 different biomolecules. In some embodiments, at least 2, 3, 4, 5, 6, 7, 8, 9, or 10 n-th plurality of distinct biomolecules each comprise at least 3, 4, 5, 6, 7, 8, 9, or 10 different biomolecules. In some embodiments, a first amount of a first biomolecule in at least one n-th plurality of distinct biomolecules and a second amount of a second biomolecule in the at least one n-th plurality of distinct biomolecules are within 1 magnitude of each other. In some embodiments, the first amount of the first biomolecule in the at least one n-th plurality of distinct biomolecules and a third amount of a third biomolecule in the at least one n-th plurality of distinct biomolecules are within 1 magnitude of each other. In some embodiments, at least one n-th peptide comprises a variant of a given peptide configured to bind to a different biomolecule in the at least three different proteins. In some embodiments, the at least one n-th peptide comprises a portion of a given binding site of the given peptide. In some embodiments, the at least one n-th peptide comprises a mutation of the given peptide such that the at least one n-th peptide comprises a modified form of the given binding site of the given peptide.

In some embodiments, the modified form comprises a different geometry than the given binding site of the given peptide. In some embodiments, the modified form comprises different vibrational modes, as measured by IR spectra or Raman spectroscopy. In some embodiments, the at least one n-th peptide comprises less specificity than a given binding site of the given peptide. In some embodiments, a first free energy of binding between the at least one n-th peptide and a first biomolecule in at least one n-th plurality of distinct biomolecules and a second free energy of binding between the at least one n-th peptide and a second biomolecule in the at least one n-th plurality of distinct biomolecules are within 1 magnitude of each other. In some embodiments, the first free energy of binding between the at least one n-th peptide and the first biomolecule in at least one n-th plurality of distinct biomolecules and a third free energy of binding between the at least one n-th peptide and a third biomolecule in the at least one n-th plurality of distinct biomolecules are within 1 magnitude of each other. In some embodiments, a first equilibrium constant of binding between the at least one n-th peptide and a first biomolecule in at least one n-th plurality of distinct biomolecules is substantially equal to a second equilibrium constant of binding between the at least one n-th peptide and a second biomolecule in the at least one n-th plurality of distinct biomolecules, as measured in an aqueous solution comprising an ITC buffer. In some embodiments, ITC buffer can maintain the solubility and stability of the biomolecules. In some embodiments, ITC buffer includes salts, cofactors, and additives for binding. In some embodiments, ITC buffer has a low enthalpy of ionization. In some embodiments, concentration of ITC buffer is high enough to compensate for any pH effects during titration. In some embodiments, the first free energy of binding between the at least one n-th peptide and the first biomolecule in at least one n-th plurality of distinct biomolecules is substantially equal to a third free energy of binding between the at least one n-th peptide and a third biomolecule in the at least one n-th plurality of distinct biomolecules, as measured in an aqueous solution comprising an ITC buffer.

In some embodiments, the at least one n-th peptide is coupled to the surface at a density of at least about 1 peptide per 50 nanometers squared. In some embodiments, the peptide is coupled to the surface at a density of at most about 1 peptide per 5 nanometers squared. In some embodiments, the peptide is coupled to the surface at a density of at most about 1 peptide per 500 nanometers squared. In some embodiments, the at least one n-th peptide is coupled to the surface at a density of at least about 1 peptide per 5 nanometers squared, 1 peptide per 10 nanometers squared, 1 peptide per 20 nanometers squared, 1 peptide per 30 nanometers squared, 1 peptide per 40 nanometers squared, 1 peptide per 50 nanometers squared, 1 peptide per 60 nanometers squared, 1 peptide per 70 nanometers squared, 1 peptide per 80 nanometers squared, 1 peptide per 90 nanometers squared, 1 peptide per 100 nanometers squared, 1 peptide per 150 nanometers squared, 1 peptide per 200 nanometers squared, 1 peptide per 250 nanometers squared, 1 peptide per 300 nanometers squared, 1 peptide per 350 nanometers squared, 1 peptide per 400 nanometers squared, 1 peptide per 450 nanometers squared, or 1 peptide per 500 nanometers squared.

In some embodiments, the at least one n-th peptide comprises at least 20 amino acids. In some embodiments, the at least one n-th peptide comprises at most 40 amino acids. In some embodiments, the at least one n-th peptide comprises at least 5 amino acids, at least 10 amino acids, at least 15 amino acids, at least 20 amino acids, at least 25 amino acids, at least 30 amino acids, at least 35 amino acids, or at least 40 amino acids. In some embodiments, the peptide comprises 5-40 amino acids, 10-35 amino acids, 15-30 amino acids, 20-25 amino acids, or 35-40 amino acids. In some embodiments, the peptide comprises at most 5 amino acids, at most 10 amino acids, at most 15 amino acids, at most 20 amino acids, at most 25 amino acids, at most 30 amino acids, at most 35 amino acids, or at most 40 amino acids.

In some embodiments, the at least one n-th peptide comprises a substantially linear domain. In some embodiments, the at least one n-th peptide comprises a non-linear domain. In some embodiments, the at least one n-th peptide comprises a circular domain. In some embodiments, at least one n-th surface further comprises a plurality of peptides coupled thereto, wherein each peptide in the plurality of peptides is configured to bind to at least three different proteins. In some embodiments, the at least three different proteins in the at least one n-th plurality of distinct biomolecules comprise the same epitope. In some embodiments, the at least three different proteins comprise at least two proteoforms expressed at least partially from the same locus of exons.

In some aspects, the present disclosure provides a method for determining a presence or absence of one or more biomolecules in the plurality of biomolecules or the portion thereof in the biological sample. In some embodiments, the method may utilize any one of the systems disclosed herein. In some embodiments, the method comprises contacting a biological sample with a surface in the system described herein to bind a plurality of biomolecules to a peptide coupled to the surface. The surface may be any one of the surfaces disclosed herein. In some embodiments, the method comprises releasing the plurality of biomolecules or a portion thereof from the surface. In some embodiments, the method comprises identifying at least the plurality of biomolecules or the portion thereof. In some embodiments, the method comprises determining a presence or absence of one or more biomolecules in the plurality of biomolecules or the portion thereof in the biological sample.

A method of the present disclosure may comprise contacting a biological sample (e.g., plasma) with a particle under conditions suitable for biomolecule collection (e.g., non-covalent adsorption) on the particle. The collection of biomolecules on the surface of the particle may be referred to as a ‘biomolecule corona’. The biomolecule corona that forms on a particle may comprise a complex mixture of biomolecules from the biological sample. The biomolecule corona may compress the abundance ratios of biomolecules from a sample, thereby enabling analysis of dilute, and in many cases difficult to analyze, biomolecules. A biomolecule corona may include nucleic acids, small molecules, proteins, lipids, polysaccharides, or any combination thereof, adsorbed to the surface of a particle form a sample in which the particle is incubated. nucleic acid, a small molecule, a protein, a lipid, a polysaccharide, or any combination thereof.

Biomolecule corona composition is often closely related to the surface properties of a particle. The present disclosure provides a range of strategies for exploiting this relationship by tailoring particle surface functionalization to promote binding to a specific subset of biomolecules from a biological sample. A particle may comprise a surface functionalization which promotes diverse and nonspecific biomolecule adsorption from a sample, and thus which may generate complex biomolecule coronas comprising large numbers of biomolecules with disparate properties. A particle may comprise a surface functionalization with an affinity for particular biomolecules from the sample, and which may thereby configure the particle to adsorb narrow ranges of biomolecules sharing common chemical, physical, and/or structural properties.

Oligopeptide surface functionalizations provide a handle for conferring degrees of binding specificity or non-specificity to a particle. Oligopeptides constitute a diverse set of biomolecules capable of achieving a wide range of physical and chemical properties. Accordingly, an oligopeptide surface functionalization may configure a particle for promiscuous biomolecule adsorption, targeted biomolecule adsorption, or any combination therein. The present disclosure provides a range of compositions, systems, and methods comprising oligopeptide functionalized particles for fractionating and analyzing biological samples.

### Particle Properties and Types

Particle types consistent with the methods disclosed herein can be made from various materials. For example, particle materials consistent with the present disclosure include metals, polymers, magnetic materials, and lipids. Magnetic particles may be iron oxide particles. Examples of metal materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron and cadmium, or any other material described in U.S. Pat. No. 7,749,299. A particle consistent with the compositions and methods disclosed herein may be a magnetic particle, such as a superparamagnetic iron oxide nanoparticle (SPION). A magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material). A particle may comprise a distinct core (e.g., the innermost portion of the particle), shell (e.g., the outermost layer of the particle), and shell or shells (e.g., portions of the particle disposed between the core and the shell). A particle may comprise a uniform composition.

A particle may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated, or may comprise a degree of phase separation. The polymer may comprise a lipid-terminated polyalkylene glycol and a polyester, or any other material disclosed in U.S. Pat. No. 9,549,901.

Examples of lipids that can be used to form the particles of the present disclosure include cationic, anionic, and neutrally charged lipids. For example, particles can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol, or any other material listed in U.S. Pat. No. 9,445,994, which is incorporated herein by reference in its entirety.

Examples of particles of the present disclosure are provided in TABLE 1.

TABLE 1
Example particles of the present disclosure

| Batch No. | Type | Particle ID | Description |
|---|---|---|---|
| S-001-001 | HX-13 | SP-001 | Carboxylate (Citrate) superparamagnetic iron oxide NPs |
| | | | (SPION) |
| S-002-001 | HX-19 | SP-002 | Phenol-formaldehyde coated SPION |
| S-003-001 | HX-20 | SP-003 | Silica-coated superparamagnetic iron oxide NPs |
| | | | (SPION) |
| S-004-001 | HX-31 | SP-004 | Polystyrene coated SPION |
| S-005-001 | HX-38 | SP-005 | Carboxylated Poly(styrene-co-methacrylic acid), P(St- |
| | | | co-MAA) coated SPION |
| S-006-001 | HX-42 | SP-006 | N-(3-Trimethoxysilylpropyl)diethylenetriamine coated |
| | | | SPION |
| S-007-001 | HX-56 | SP-007 | poly(N-(3-(dimethylamino)propyl) methacrylamide) |
| | | | (PDMAPMA)-coated SPION |
| S-008-001 | HX-57 | SP-008 | 1,2,4,5-Benzenetetracarboxylic acid coated SPION |
| S-009-001 | HX-58 | SP-009 | PVBTMAC coated |
| | | | poly(vinylbenzyltrimethylammonium chloride) |
| | | | (PVBTMAC) coated SPION |
| S-010-001 | HX-59 | SP-010 | Carboxylate, PAA coated SPION |
| S-011-001 | HX-86 | SP-011 | poly(oligo(ethylene glycol) methyl ether methacrylate) |
| | | | (POEGMA)-coated SPION |
| S-163-001 | | S-163 | Cis-ubiquitin-functionalized styrene particle |
| S-164-001 | | S-164 | Ubiquitin-functionalized styrene particle |
| P-033-001 | P33 | SP-333 | Carboxylate functionalized 1 μm magnetic |
| | | | microparticle, surfactant free SPION |
| P-039-003 | P39 | SP-339 | Polystyrene carboxyl functionalized SPION |
| P-041-001 | P41 | SP-341 | Carboxylic acid SPION |
| P-047-001 | P47 | SP-365 | Silica SPION |
| P-048-001 | P48 | SP-348 | Carboxylic acid, 150 nm SPION |
| P-053-001 | P53 | SP-353 | Amino surface microparticle, 0.4-0.6 μm SPION |
| P-056-001 | P56 | SP-356 | Silica amino functionalized microparticle, 0.1-0.39 μm |
| | | | SPION |
| P-063-001 | P63 | SP-363 | Jeffamine surface, 0.1-0.39 μm SPION |
| P-064-001 | P64 | SP-364 | Polystyrene microparticle, 2.0-2.9 μm SPION |
| P-065-001 | P65 | SP-365 | Silica SPION |
| P-069-001 | P69 | SP-369 | Carboxylated Original coating, 50 nm SPION |
| P-073-001 | P73 | SP-373 | Dextran based coating, 0.13 μm SPION |
| P-074-001 | P74 | SP-374 | Silica Silanol coated with lower acidity SPION |

A particle of the present disclosure may be synthesized, or a particle of the present disclosure may be purchased from a commercial vendor. For example, particles consistent with the present disclosure may be purchased from commercial vendors including Sigma-Aldrich, Life Technologies, Fisher Biosciences, nanoComposix, Nanopartz, Spherotech, and other commercial vendors. In some embodiments, a particle of the present disclosure may be purchased from a commercial vendor and further modified, coated, or functionalized.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity.

A particle may be provided at a range of concentrations. A particle may comprise a concentration between 100 fM and 100 nM. A particle may comprise a concentration between 100 fM and 10 pM. A particle may comprise a concentration between 1 pM and 100 pM. A particle may comprise a concentration between 10 PM and 1 nM. A particle may comprise a concentration between 100 pM and 10 nM. A particle may comprise a concentration between 1 nM and 100 nM. A particle may be contacted to a biological sample at a ratio of volume ratios. A solution comprising a particle may be combined with a biological sample, at a volume ratio of greater than about 100:1, about 100:1, about 80:1, about 60:1, about 50:1, about 40:1, about 30:1, about 25:1, about 20:1, about 15:1, about 12:1, about 10:1, about 8:1, about 6:1, about 5:1, about 4:1, about 3:1, about 5:2, about 2:1, about 3:2, about 1:1, about 2:3, about 1:2, about 2:5, about 1:3, about 1:4, about 1:5, about 1:6, about 1:8, about 1:10, about 1:12, about 1:15, about 1:20, about 1:25, about 1:30, about 1:40, about 1:50, about 1:60, about 1:80, about 1:100, or less than about 1:100.

Particles that are consistent with the present disclosure can comprise a wide range of sizes. In some embodiments, a particle of the present disclosure may be a nanoparticle. In some embodiments, a nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some embodiments, a nanoparticle may be less than 1000 nm in diameter.

A particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. In some embodiments, a microparticle may be less than 1000 μm in diameter.

The ratio between surface area and mass can be a determinant of a particle's properties in the methods of the instant disclosure. For example, the number and types of biomolecules that a particle adsorbs from a solution may vary with the particle's surface area to mass ratio. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have higher surface area to mass ratios than large particles (e.g., with diameters of 200 nm or more). In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg.

In some embodiments, a plurality of particles (e.g., of a particle panel) of the compositions and methods described herein may comprise a range of surface area to mass ratios. In some embodiments, the range of surface area to mass ratios for a plurality of particles is less than 100 cm²/mg, 80 cm²/mg, 60 cm²/mg, 40 cm²/mg, 20 cm²/mg, 10 cm²/mg, 5 cm²/mg, or 2 cm²/mg. In some embodiments, the surface area to mass ratios for a plurality of particles varies by no more than 40%, 30%, 20%, 10%, 5%, 3%, 2%, or 1% between the particles in the plurality.

In some embodiments, a plurality of particles (e.g., in a particle panel) may have a wider range of surface area to mass ratios. In some embodiments, the range of surface area to mass ratios for a plurality of particles is greater than 100 cm²/mg, 150 cm²/mg, 200 cm²/mg, 250 cm²/mg, 300 cm²/mg, 400 cm²/mg, 500 cm²/mg, 800 cm²/mg, 1000 cm²/mg, 1200 cm²/mg, 1500 cm²/mg, 2000 cm²/mg, 3000 cm²/mg, 5000 cm²/mg, 7500 cm²/mg, 10000 cm²/mg, or more. In some embodiments, the surface area to mass ratios for a plurality of particles (e.g., within a panel) can vary by more than 100%, 200%, 300%, 400%, 500%, 1000%, 10000% or more. In some embodiments, the plurality of particles with a wide range of surface area to mass ratios comprises at least 2, 3, 4, 5, 6, 7, 8, 9, 10, 12, 15, 20, or more different types of particles.

A particle may comprise a wide array of physical properties. A physical property of a particle may include composition, size, surface charge, hydrophobicity, hydrophilicity, surface functionalization, surface topography, surface curvature, porosity, core material, shell material, shape, and any combination thereof.

A surface functionalization may comprise a polymerizable functional group, a positively or negatively charged functional group, a zwitterionic functional group, an acidic or basic functional group, a polar functional group, or any combination thereof. A surface functionalization may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. A particle from among the plurality of particles may be selected from the group consisting of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, and 2-(3-aminopropylamino)ethanol particles, and any combination thereof.

Particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

The present disclosure includes compositions (e.g., particle panels) and methods that comprise two or more particles differing in at least one physicochemical property. A composition or method of the present disclosure may comprise 3 to 6 particles differing in at least one physicochemical property. A composition or method of the present disclosure may comprise 4 to 8 particles differing in at least one physicochemical property. A composition or method of the present disclosure may comprise 4 to 10 particles differing in at least one physicochemical property. A composition or method of the present disclosure may comprise 5 to 12 particles differing in at least one physicochemical property. A composition or method of the present disclosure may comprise 6 to 14 particles differing in at least one physicochemical property. A composition or method of the present disclosure may comprise 8 to 15 particles differing in at least one physicochemical property. A composition or method of the present disclosure may comprise 10 to 20 particles differing in at least one physicochemical property. A composition or method of the present disclosure may comprise at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 20 distinct particle types, at least 25 particle types, or at least 30 distinct particle types.

A particle of the present disclosure may be contacted with a biological sample (e.g., a biofluid) to form a biomolecule corona. The particle and biomolecule corona may be separated from the biological sample, for example by centrifugation, ultracentrifugation, density or gradient-based centrifugation, magnetic separation, filtration, chromatographic separation, gravitational separation, charge-based separation, column-based separation, spin column-based separation, or any combination thereof. Each of a plurality of particle types may be separated from a biological sample or from a mixture of particles based on their physical, chemical, charge, or magnetic properties. Protein corona analysis may be performed on the separated particle and biomolecule corona. Protein corona analysis may comprise identifying one or more proteins in the biomolecule corona, for example by mass spectrometry. A single particle type (e.g., a particle of a type listed in TABLE 1) may be contacted to a biological sample. A plurality of particle types (e.g., a plurality of the particle types provided in TABLE 1) may be contacted to a biological sample. The plurality of particle types may be combined and contacted to the biological sample in a single sample volume. The plurality of particle types may be sequentially contacted to a biological sample and separated from the biological sample prior to contacting a subsequent particle type to the biological sample. Protein corona analysis of the biomolecule corona may compress the dynamic range of the analysis compared to a total protein analysis method.

The particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. Serial interrogation may also comprise collecting biomolecules of a biomolecule corona from a first particle, and contacting the biomolecules to a second particle to form a second biomolecule corona. In some embodiments, the biomolecule corona on each particle type used for serial interrogation of a sample may be analyzed by protein corona analysis. The biomolecule content of the supernatant may be analyzed following serial interrogation with one or more particle types.

### Binding Molecule Particle Functionalizations

A particle's surface functionalization can influence the composition of the particle's biomolecule corona. Surface functionalizations can include small molecule functionalizations (e.g., a monosaccharide or terpene), oligomeric functionalizations (e.g., oligonucleotides or oligopeptides), or macromolecular functionalizations (e.g., proteins). A surface functionalization may be directly or indirectly coupled to a particle. For example, a surface functionalization may be covalently coupled to a metal oxide or polymeric particle surface, or may be coupled to a particle by a linker. A surface functionalization may comprise or be coupled to additional surface functionalizations. A surface functionalization may comprise a chemical modification that affects its physical, chemical, or analyte binding properties. For example, an oligopeptide surface functionalization may comprise a post-translational modification (or a chemical functionalization mimetic of a post-translational modification), such as tyrosine phosphorylation.

A surface functionalization may comprise a binding molecule. The binding molecule may be a small molecule, an oligomer, or a macromolecule. The binding molecule may comprise an binding specificity for a group or class of analytes (e.g., a plurality of saccharides or a class of proteins). A binding molecule may comprise a moderate binding specificity for the group or class of analytes. Conversely, a binding molecule may comprise a dis-affinity for a group or class of analytes, disfavoring binding of these species relative to the same particle lacking the binding molecule. For example, a binding molecule may comprise a negative charge distribution which repels negatively charged nucleic acids, thereby disfavoring their binding.

A binding molecule may comprise a range of binding specificities for analytes from a sample. As binding specificity may be a complex function of analyte-particle binding strength and the combination of on-particle and solution phase analyte-analyte binding strengths, the binding specificity of a binding molecule may be measured as the binding strength (e.g., dissociation constant, K_{D}) between the binding molecule and an isolated, purified form of the analyte; the binding strength between the binding molecule and the analyte present in a complex mixture (e.g., the sample from which it is derived); or the binding strength between the analyte and a particle comprising the binding molecule. A binding molecule or a plurality of binding molecules may comprise a binding specificity of at least 10 M (thereby disfavoring binding), at least 1 M, at least 100 mM, at least 10 mM, at least 1 mM (e.g., measured as a K_{D}), at least 100 μM, at least 10 μM, at least 1 μM, at least 100 nM, at least 10 nM, at least 1 nM, or at least 100 pM for a plurality of analytes. A binding molecule or a plurality of binding molecules may comprise a binding specificity of at most 10 mM, at most 1 mM (e.g., measured as a K_{D}), at most 100 μM, at most 10 μM, at most 1 μM, at most 100 nM, at most 10 nM, at most 1 nM, or at most 100 pM for a plurality of analytes. A binding molecule or a plurality of binding molecules may comprise a binding specificity for at least 2, at least 3, at least 4, at least 5, at least 6, at least 7, at least 8, at least 9, at least 10, at least 12, at least 15, at least 20, at least 25, at least 30, at least 35, at least 40, at least 50, at least 60, at least 80, at least 100, at least 120, at least 150, at least 180, at least 200, at least 250, at least 300, at least 400, or at least 500 analytes (e.g., from among all known species or from among the species present in a particular sample). For example, a binding molecule may comprise a binding affinity of 100 μM or less for 35 different plasma proteins. A binding molecule or a plurality of binding molecules may comprise a binding specificity for, at most 3, at most 4, at most 5, at most 6, at most 7, at most 8, at most 9, at most 10, at most 12, at most 15, at most 20, at most 25, at most 30, at most 35, at most 40, at most 50, at most 60, at most 80, at most 100, at most 120, at most 150, at most 180, at most 200, at most 250, at most 300, at most 400, or at most 500 analytes. A binding molecule may comprise a binding specificity for at least 2, at least 3, at least 4, at least 5, at least 6, at least 7, at least 8, at least 9, at least 10, at least 12, at least 15, at least 20, at least 25, at least 30, at least 35, at least 40, at least 50, at least 60, at least 80, at least 100, at least 120, at least 150, at least 180, at least 200, at least 250, at least 300, at least 400, or at least 500 proteins (e.g., from among all known proteins or from among the proteins present in a particular sample). A binding molecule or a plurality of binding molecules may have a binding specificity for at most 3, at most 4, at most 5, at most 6, at most 7, at most 8, at most 9, at most 10, at most 12, at most 15, at most 20, at most 25, at most 30, at most 35, at most 40, at most 50, at most 60, at most 80, at most 100, at most 120, at most 150, at most 180, at most 200, at most 250, at most 300, at most 400, or at most 500 proteins. A binding molecule or a plurality of binding molecules may comprise a binding affinity for between 1 and 5 different analytes, between 2 and 10 different analytes, between 5 and 10 different analytes, between 5 and 20 different analytes, between 5 and 30 different analytes, between 5 and 50 different analytes, between 10 and 20 different analytes, between 10 and 30 different analytes, between 10 and 50 different analytes, between 10 and 100 different analytes, between 10 and 200 different analytes, between 10 and 300 different analytes, between 10 and 500 different analytes, between 20 and 40 different analytes, between 20 and 50 different analytes, between 20 and 80 different analytes, between 20 and 100 different analytes, between 20 and 200 different analytes, between 20 and 300 different analytes, between 20 and 500 different analytes, between 30 and 60 different analytes, between 30 and 80 different analytes, between 30 and 100 different analytes, between 30 and 150 different analytes, between 30 and 200 different analytes, between 30 and 250 different analytes, between 30 and 500 different analytes, between 40 and 100 different analytes, between 40 and 200 different analytes, between 50 and 100 different analytes, between 50 and 200 different analytes, between 50 and 300 different analytes, between 50 and 400 different analytes, between 80 and 150 different analytes, between 80 and 200 different analytes, between 80 and 300 different analytes, between 80 and 400 different analytes, between 100 and 200 different analytes, between 100 and 400 different analytes, or between 200 and 400 different analytes. A binding molecule or a plurality of binding molecules may comprise a binding affinity for between 1 and 5 different proteins, between 2 and 10 different proteins, between 5 and 10 different proteins, between 5 and 20 different proteins, between 5 and 30 different proteins, between 5 and 50 different proteins, between 10 and 20 different proteins, between 10 and 30 different proteins, between 10 and 50 different proteins, between 10 and 100 different proteins, between 10 and 200 different proteins, between 10 and 300 different proteins, between 10 and 500 different proteins, between 20 and 40 different proteins, between 20 and 50 different proteins, between 20 and 80 different proteins, between 20 and 100 different proteins, between 20 and 200 different proteins, between 20 and 300 different proteins, between 20 and 500 different proteins, between 30 and 60 different proteins, between 30 and 80 different proteins, between 30 and 100 different proteins, between 30 and 150 different proteins, between 30 and 200 different proteins, between 30 and 250 different proteins, between 30 and 500 different proteins, between 40 and 100 different proteins, between 40 and 200 different proteins, between 50 and 100 different proteins, between 50 and 200 different proteins, between 50 and 300 different proteins, between 50 and 400 different proteins, between 80 and 150 different proteins, between 80 and 200 different proteins, between 80 and 300 different proteins, between 80 and 400 different proteins, between 100 and 200 different proteins, between 100 and 400 different proteins, or between 200 and 400 different proteins.

Particle multiplexing is often limited by high abundance analyte saturation. When a plurality of particles collectively enriches a common set of high abundance analytes from a sample, the collective signals from the high abundance analytes often diminish signal resolution from lower abundance analytes of interest. Binding molecule functionalizations provide a means for circumventing this issue, by enabling differential high abundance analyte collection across a range of particles. A plurality of particles may comprise a plurality of binding molecule functionalizations which promote binding by separate groups of high abundance analytes. Rather than concentrating a particular set of high abundance analytes, such a plurality of particles may selectively enrich a plurality of low abundance analytes (e.g., 200 low abundance analytes) from the sample, increasing their abundances relative to those of high abundance analytes from the sample. Two particles may comprise at most 95%, at most 90%, at most 85%, at most 80%, at most 75%, at most 70%, at most 65%, at most 60%, at most 55%, at most 50%, at most 45%, at most 40%, at most 35%, at most 30%, at most 25%, at most 20%, at most 15%, at most 10%, or at most 5% overlap between the sets of analytes for which they have binding specificities. Two particles may comprise at most 95%, at most 90%, at most 85%, at most 80%, at most 75%, at most 70%, at most 65%, at most 60%, at most 55%, at most 50%, at most 45%, at most 40%, at most 35%, at most 30%, at most 25%, at most 20%, at most 15%, at most 10%, or at most 5% overlap between the sets of proteins for which they have binding specificities. Two particles may comprise at most 95%, at most 90%, at most 85%, at most 80%, at most 75%, at most 70%, at most 65%, at most 60%, at most 55%, at most 50%, at most 45%, at most 40%, at most 35%, at most 30%, at most 25%, at most 20%, at most 15%, at most 10%, or at most 5% overlap between the sets high abundance proteins for which they have binding specificities.

A binding molecule may comprise a small molecule, an oligomer, a macromolecule, or a combination thereof. A macromolecular binding molecule may comprise a biomacromolecule, such as a protein or a nucleic acid (e.g., a 100-mer DNA molecule). A macromolecular binding molecule may comprise a protein, polynucleotide, or polysaccharide, or may be comparable in size to any of such species. For example, a macromolecular binding molecule may comprise a volume of at least 6 nm³, at least 8 nm³, at least 12 nm³, at least 15 nm³, at least 20 nm³, at least 30 nm³, at least 50 nm³, at least 80 nm³, at least 120 nm³, at least 180 nm³, at least 300 nm³, at least 500 nm³, at least 800 nm³, at least 1200 nm³, at least 1500 nm³, or at least 2000 nm³. A macromolecular functionalization may comprise a surface area of at least at least 15 nm², at least 20 nm², at least 25 nm², at least 40 nm², at least 80 nm², at least 150 nm², at least 300 nm², at least 500 nm², at least 800 nm², at least 1200 nm², or at least 1500 nm².

A binding molecule may be directly attached to a particle (e.g., covalently bound to a surface oxide or surface polymer of a particle) or may be tethered to a particle via a linker. The linker may hold the macromolecule close to the particle, thereby restricting its motion and reorientation relative to the particle, or may extend the binding molecule away from the particle, providing a range of conformational and translational freedom. The linker may be rigid (e.g., a polyolefin linker) or flexible (e.g., a nucleic acid linker). A linker may be no more than 0.5 nm in length, no more than 1 nm in length, no more than 1.5 nm in length, no more than 2 nm in length, no more than 3 nm in length, no more than 4 nm in length, no more than 5 nm in length, no more than 8 nm in length, or no more than 10 nm in length. A linker may be at least 1 nm in length, at least 2 nm in length, at least 3 nm in length, at least 4 nm in length, at least 5 nm in length, at least 8 nm in length, at least 12 nm in length, at least 15 nm in length, at least 20 nm in length, at least 25 nm in length, or at least 30 nm in length. As such, a surface functionalization on a particle may project beyond a primary corona associated with the particle. A surface functionalization may also be situated beneath or within a biomolecule corona that forms on the particle surface.

A binding molecule may comprise specific or variable forms of coupling to a particle. A plurality of binding molecules of a particular type may comprise a single type of linkage to a particle, or may comprise a plurality of linkage types to the particle. For example, a protein or oligopeptide may be coupled to a particle by its C-terminus, or may be tethered to a particle by any number of amino acid residue side chains. For example, a peptide may be covalently attached to a particle via any of its lysine butylamine side chains. A binding molecule may comprise more than one linkage to a particle. For example, an oligopeptide binding molecule may be attached to a particle by both its C-terminus and its N-terminus.

The present disclosure provides a range of linkers for coupling a peptide to a particle. A linker may comprise a first reactive moiety configured to couple to a peptide and a second reactive moiety configured to couple to a particle or to a reactive moiety coupled to the particle. A first reactive moiety may be configured to couple to an N-terminal amino acid, a C-terminal amino acid, an internal amino acid, or a derivative thereof. A first reactive moiety for coupling to a peptide N-terminus may comprise a ketene, a pyridinecarboxyaldehyde, a maleimide, an activated ester, a Michael acceptor, or any combination thereof. A first reactive moiety for coupling to a peptide C-terminus may comprise a carbodiimide, an isoxazolium, or any combination thereof. A first reactive moiety for coupling to an internal amino acid may comprise an epoxide (e.g., for coupling to a histidine side chain), an alpha-ketoaldehyde (e.g., for coupling to an arginine side chain), an isothiocyanate, an activated ester (e.g., for coupling to a lysine or cysteine side chain), an azide (e.g., for coupling to an aromatic side chain moiety), an organoiodine (e.g., for coupling to a cysteine side chain), a propiolamide (e.g., for coupling to a cysteine side chain), or any combination thereof.

A first reactive moiety may be configured to couple to an activated amino acid. A first reactive moiety may comprise a nucleophile, such as a thiol, configured to couple to a periodate-derived glyoxylate N-terminal functionalization. A first reactive moiety may comprise a nucleophile configured to couple to an alpha-ketoacid-derived alpha-diketone N-terminal functionalization. A first reactive moiety may comprise a nucleophile configured to couple to an esterified or activated (e.g., activated by a uronium salt such as Hexafluorophosphate Azabenzotriazole Tetramethyl Uronium (HATU)) C-terminal or side chain carboxylate. A first reactive moiety may comprise an oxime for coupling to an N-terminal, C-terminal, or internal amino acid carbonyl. A first reactive moiety may comprise an azide for coupling to an N-terminal, a C-terminal, or an internal amino acid functionalization. A first reactive group may comprise a nucleophile, such as a primary amine, configured to couple to a Lewis acid-activated C-terminal.

A second reactive moiety may comprise an electrophilic group for coupling to a particle-derived nucleophile. A second reactive moiety may comprise an oxirane, an N-Hydroxysuccinimide (NHS) ester, a maleimide, a uranium salt-activated carboxylate, such as a Hexafluorophosphate Benzotriazole Tetramethyl Uronium (HBTU) activated carboxylate, an isocyante, or any combination thereof for coupling to a particle-derived nucleophile. For example, a second reactive moiety may comprise a maleimide for coupling to a particle-derived amine. A second reactive moiety may comprise a nucleophilic group, such as an azide, a hydrazine, or an amine for coupling to a particle-derived electrophile, such as an olefin, ester, aldehyde, or activated carboxylate. A second reactive group may comprise a azide or an alkyne for click-chemistry based coupling to a particle derived alkyne or azide. A second reactive moiety may comprise an activatable group, such as a vinyl halogen, configured to couple (e.g., catalyst-mediated cross couple) to a particle-derived moiety. A second reactive moiety may comprise a diene or a dienophile for Diels-Alder addition to a particle derived olefin.

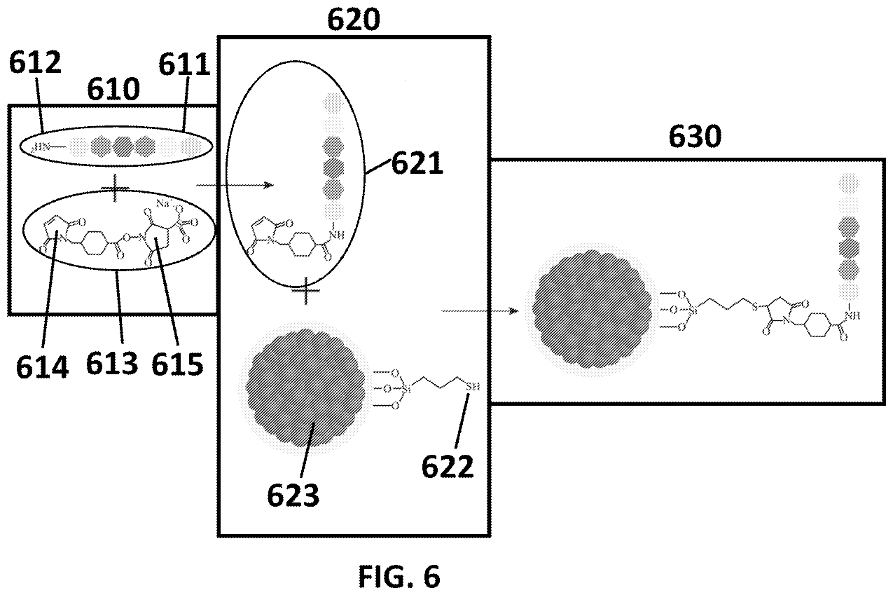

FIG. 6 provides an example of a method for coupling a peptide-containing binding molecule to a particle by a linker. A peptide 611 comprising an N-terminus 612 may be combined with a bifunctional linker 613 comprising a first reactive moiety 614 and a second reactive moiety 615, for example an 4-(N-Maleimidomethyl)cyclohexane-1-carboxylic acid 3-sulfo-N-hydroxysuccinimide ester (SMCC) comprising an N-hydroxysuccinimide ester as a first reactive moiety 614 and a maleimide group as a second reactive moiety 615. The first reactive moiety of the bifunctional linker may be coupled 620 to the peptide, for example at its N-terminus 612, yielding a peptide-linker conjugate 621. The second reactive moiety 615 of the bifunctional linker may then be coupled 630 to a reactive group 622 of a particle 623, such as a thiol, thereby coupling 630 the peptide-linker conjugate to the surface of the particle. Alternatively, the first reactive moiety of the bifunctional linker may be coupled to the reactive group of the particle, and then coupled to the peptide.

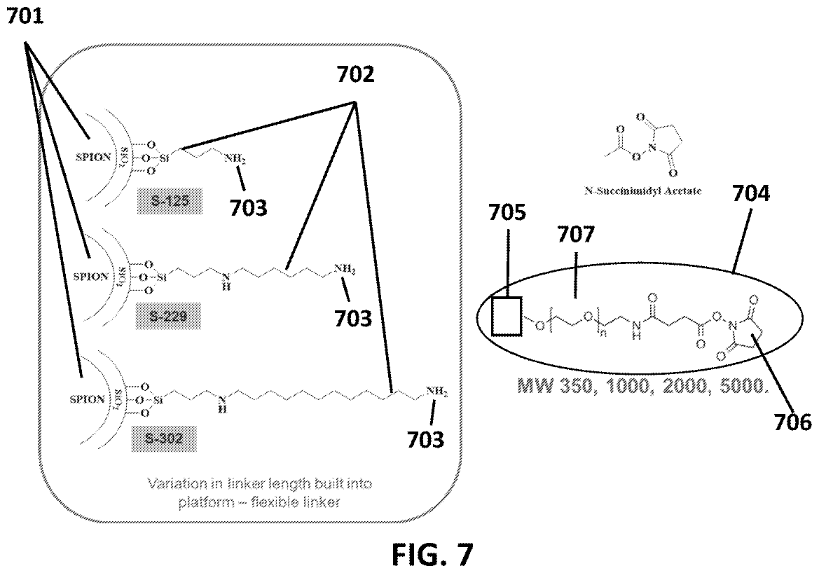

FIG. 7 provides examples of linkers of differing lengths which may be used to couple a binding molecule to a particle. The particle 701 may comprise various length linkers 702 between the particle surface and a reactive handle 703. A binding molecule (e.g., a peptide) may be coupled to the particle surface by a bifunctional linker 704 comprising a first site 705 for coupling to a particle reactive handle 703 and a second group for coupling to a peptide 706. The bifunctional linker may diminish, maintain, or extend the average distance between a binding molecule and a particle. For example, a multimeric linker backbone 707 may serve to further separate a binding molecule from a particle.

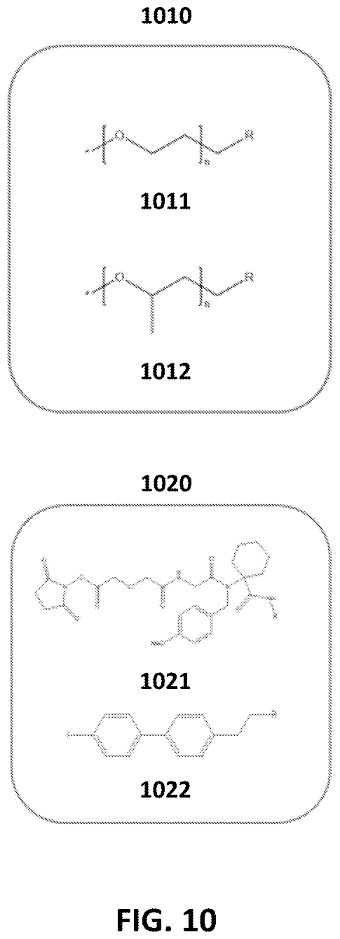

FIG. 10 provides examples of linkers with differing degrees of flexibility. A linker may comprise a high degree of flexibility. A flexible linker 1010 may comprise a backbone structure with rotationally unconstrained bonds, such as carbon-carbon and carbon-oxygen single bonds. Examples of flexible linkers include polyethylene glycol 1011 and polypropylene oxide 1012. A linker may comprise a degree of rigidity. A rigid linker 1020 may comprise semi-rigid peptide bonds 1021, aromatic groups configured for π-π stacking 1022, or a backbone double or triple bond. A linker may comprise a rigid portion and a flexible portion. A linker may comprise a defined secondary or tertiary structure.

A particle may comprise a binding molecule (e.g., a single type of oligopeptide) or a plurality of binding molecules. A particle may comprise a single type of binding molecule, 2 types of binding molecules, 3 types of binding molecules, 4 types of binding molecules, 5 types of binding molecules, 6 types of binding molecules, 7 types of binding molecules, 8 types of binding molecules, 9 types of binding molecules, 10 types of binding molecules, 11 types of binding molecules, 12 types of binding molecules, 15 types of binding molecules, 20 types of binding molecules, 25 types of binding molecules, 30 types of binding molecules, 40 types of binding molecules, 50 types of binding molecules, or greater than 50 types of binding molecules. A particle may comprise at least 1 type of binding molecule, at least 2 types of binding molecules, at least 3 types of binding molecules, at least 4 types of binding molecules, at least 5 types of binding molecules, at least 6 types of binding molecules, at least 7 types of binding molecules, at least 8 types of binding molecules, at least 9 types of binding molecules, at least 10 types of binding molecules, at least 11 types of binding molecules, at least 12 types of binding molecules, at least 15 types of binding molecules, at least 20 types of binding molecules, at least 25 types of binding molecules, at least 30 types of binding molecules, at least 40 types of binding molecules, or at least 50 types of binding molecules. A particle may comprise at most 1 type of binding molecule, at most 2 types of binding molecules, at most 3 types of binding molecules, at most 4 types of binding molecules, at most 5 types of binding molecules, at most 6 types of binding molecules, at most 7 types of binding molecules, at most 8 types of binding molecules, at most 9 types of binding molecules, at most 10 types of binding molecules, at most 11 types of binding molecules, at most 12 types of binding molecules, at most 15 types of binding molecules, at most 20 types of binding molecules, at most 25 types of binding molecules, at most 30 types of binding molecules, at most 40 types of binding molecules, or at most 50 types of binding molecules. A particle may comprise 1 to 5 types of binding molecules. A particle may comprise 2 to 5 types of binding molecules. A particle may comprise 3 to 5 types of binding molecules. A particle may comprise 4 to 5 types of binding molecules. A particle may comprise 1 to 4 types of binding molecules. A particle may comprise 2 to 4 types of binding molecules. A particle may comprise 3 to 4 types of binding molecules. A particle may comprise 1 to 3 types of binding molecules. A particle may comprise 2 to 3 types of binding molecules. A particle may comprise a controlled ratio of two or more types of binding molecules. For example, a particle may comprise 3 distinct types of oligopeptides in a ratio of 15:4:1. A plurality of types of binding molecules may be evenly distributed across a particle surface. A plurality of types of binding molecules may comprise a degree of patterning or separation over a particle surface. For example, a particle may comprise a first oligopeptide surface functionalization over a first hemisphere of its surface and a second oligopeptide surface functionalization over a second hemisphere of its surface.

A particle may comprise a range of binding molecule densities on its surface. A particle may comprise about 1 to about 10, about 10 to about 50, about 50 to about 10², about 10² to about 5×10², about 5×10² to about 10³, about 10³ to about 5×10³, about 5×10³ to about 10⁴, about 10⁴ to about 5×10⁴, about 5×10⁴ to about 10⁵, about 10⁵ to about 5×10⁵, about 5×10⁵ to about 10⁶, about 10⁶ to about 5×10⁶, about 5×10⁶ to about 10⁷, at least about 10², at least about 10³, at least about 10⁴, at least about 10⁵, at least about 10⁶, at least about 10⁷, at most about 10⁷, at most about 10⁶, at most about 10⁵, at most about 10⁴, at most about 10³, or at most about 10² binding molecules coupled to its surface. A particle may comprise an average spacing between surface-bound binding molecules of at least about 0.5 nm, at least about 1 nm, at least about 1.5 nm, at least about 2 nm, at least about 2.5 nm, at least about 3 nm, at least about 4 nm, at least about 5 nm, at least about 6 nm, at least about 8 nm, at least about 10 nm, at least about 12 nm, at least about 15 nm, at least about 20 nm, at least about 25 nm, at most about 25 nm, at most about 20 nm, at most about 15 nm, at most about 12 nm, at most about 10 nm, at most about 8 nm, at most about 6 nm, at most about 5 nm, at most about 4 nm, at most about 3 nm, at most about 2.5 nm, at most about 2 nm, at most about 1.5 nm, at most about 1 nm, or at most about 0.5 nm. A particle may comprise a binding molecule surface density of at least about 5 binding molecules per nm² particle surface area, at least about 3 binding molecules per nm² particle surface area, at least about 2 binding molecules per nm² particle surface area, at least about 1 binding molecule per nm² particle surface area, at least about 1 binding molecule per 1.5 nm² particle surface area, at least about 1 binding molecule per 2 nm² particle surface area, at least about 1 binding molecule per 2.5 nm² particle surface area, at least about 1 binding molecule per 3 nm² particle surface area, at least about 1 binding molecule per 4 nm² particle surface area, at least about 1 binding molecule per 5 nm² particle surface area, at least about 1 binding molecule per 6 nm² particle surface area, at least about 1 binding molecule per 8 nm² particle surface area, at least about 1 binding molecule per 10 nm² particle surface area, at least about 1 binding molecule per 12 nm² particle surface area, at least about 1 binding molecule per 15 nm² particle surface area, at least about 1 binding molecule per 20 nm² particle surface area, at least about 1 binding molecule per 25 nm² particle surface area, at least about 1 binding molecule per 30 nm² particle surface area, at least about 1 binding molecule per 40 nm² particle surface area, at least about 1 binding molecule per 50 nm² particle surface area, at least about 1 binding molecule per 75 nm² particle surface area, at least about 1 binding molecule per 100 nm² particle surface area, at least about 1 binding molecule per 150 nm² particle surface area, at least about 1 binding molecule per 200 nm² particle surface area, at least about 1 binding molecule per 250 nm² particle surface area, at least about 1 binding molecule per 300 nm² particle surface area, at least about 1 binding molecule per 400 nm² particle surface area, at least about 1 binding molecule per 500 nm² particle surface area, at least about 1 binding molecule per 600 nm² particle surface area, or at least about 1 binding molecule per 800 nm² particle surface area, or at least about 1 binding molecule per 1000 nm² particle surface area. A particle may comprise a binding molecule surface functionalization density of at most about 5 binding molecules per nm² particle surface area, at most about 3 binding molecules per nm² particle surface area, at most about 2 binding molecules per nm² particle surface area, at most about 1 binding molecule per nm² particle surface area, at most about 1 binding molecule per 1.5 nm² particle surface area, at most about 1 binding molecule per 2 nm² particle surface area, at most about 1 binding molecule per 2.5 nm² particle surface area, at most about 1 binding molecule per 3 nm² particle surface area, at most about 1 binding molecule per 4 nm² particle surface area, at most about 1 binding molecule per 5 nm² particle surface area, at most about 1 binding molecule per 6 nm² particle surface area, at most about 1 binding molecule per 8 nm² particle surface area, at most about 1 binding molecule per 10 nm² particle surface area, at most about 1 binding molecule per 12 nm² particle surface area, at most about 1 binding molecule per 15 nm² particle surface area, at most about 1 binding molecule per 20 nm² particle surface area, at most about 1 binding molecule per 25 nm² particle surface area, at most about 1 binding molecule per 30 nm² particle surface area, at most about 1 binding molecule per 40 nm² particle surface area, at most about 1 binding molecule per 50 nm² particle surface area, at most about 1 binding molecule per 75 nm² particle surface area, at most about 1 binding molecule per 100 nm² particle surface area, at most about 1 binding molecule per 150 nm² particle surface area, at most about 1 binding molecule per 200 nm² particle surface area, at most about 1 binding molecule per 250 nm² particle surface area, at most about 1 binding molecule per 300 nm² particle surface area, at most about 1 binding molecule per 400 nm² particle surface area, at most about 1 binding molecule per 500 nm² particle surface area, at most about 1 binding molecule per 600 nm² particle surface area, or at most about 1 binding molecule per 800 nm² particle surface area, or at most about 1 binding molecule per 1000 nm² particle surface area. A particle may comprise sufficiently dense binding molecule surface functionalizations to prevent or minimize direct interaction (e.g., contact) between its surface and analytes within its biomolecule corona. Alternatively, a particle may comprise sufficiently low density binding molecule surface functionalizations so as to permit direct interaction (e.g., contact) between its surface and analytes of its biomolecule corona.

A binding molecule may comprise a small molecule. A small molecule may comprise a mass of fewer than 600 Daltons, fewer than 500 Daltons, fewer than 400 Daltons, fewer than 300 Daltons, fewer than 200 Daltons, or fewer than 100 Daltons. A small molecule may comprise an ionizable moiety, such as a chemical group with a pKₐ or pK_{b} of less than 6 or 7. A small molecule may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

### Oligopeptide Functionalizations

A binding molecule may comprise a peptide. Peptides are an extensive and diverse set of biomolecules which may comprise a wide range of physical and chemical properties. Depending on its composition, sequence, and chemical modification, a peptide may be hydrophilic, hydrophobic, amphiphilic, lipophilic, lipophobic, positively charged, negatively charged, zwitterionic, neutral, chaotropic, antichaotropic, reactive, redox active, inert, acidic, basic, rigid, flexible, or any combination thereof. Accordingly, a peptide surface functionalization may confer a range of physicochemical properties to a particle.

A particle may comprise a single peptide surface functionalization or a plurality of peptide surface functionalizations. A single peptide surface functionalization may comprise a plurality of identical or sequence-sharing peptides bound to a particle in a uniform fashion. For example, a particle comprising a single peptide surface functionalization may comprise about 3×10⁵ peptides of the sequence alanine-valine-tyrosine-proline-histidine-phosphotyrosine-hydroxyproline-phenylalanine-tryptophan-alanine-arginine, each coupled by its C-terminal arginine to the particle surface.

A plurality of peptide surface functionalizations may comprise a plurality of peptides sharing a common sequence but bound to a particle in a plurality of fashions. For example, a plurality of identical peptides sharing a common sequence of alanine-lysine-alanine-lysine-alanine-lysine-proline may provide a plurality of surface functionalizations to a single particle when separately coupled to the particle through any one of the lysine residues. A plurality of peptide surface functionalizations may comprise a plurality of peptides sharing common sequence but bearing different chemical modifications. For example, a particle may comprise a plurality of peptide surface functionalizations sharing a common sequence but differing in N-terminal functionalization. A plurality of peptide surface functionalizations may comprise peptides with different lengths or sequences.

A particle may comprise any number of peptide surface functionalizations. A particle may comprise a single peptide surface functionalization. A particle may comprise at least 2, at least 3, at least 4, at least 5, at least 6, at least 8, at least 10, at least 12, at least 15, at least 20, at least 25, at least 30, at least 40, at least 50, at least 80, at least 100, at least 150, at least 200, at least 250, at least 500, at least 10³, at least 2×10³, at least 5×10³, at least 10⁴, at least 5×10⁴, at least 10⁵, at least 5×10⁵, or at least 10⁶ types of peptide surface functionalizations. A particle may comprise at most 10⁶, at most 5×10⁵, at most 10⁵, at most 5×10⁴, at most 10⁴, at most 5×10³, at most 10³, at most 500, at most 250, at most 200, at most 150, at most 100, at most 80, at most 50, at most 40, at most 30, at most 25, at most 20, at most 15, at most 12, at most 10, at most 8, at most 6, at most 5, at most 4, at most 3, or at most 2 types of peptide surface functionalizations. In some embodiments, each peptide surface functionalization of a particle is unique. Such a diversity of surface functionalizations may be achieved, for example, through relatively facile combinatorial peptide synthesis. As peptide sequence diversity grows exponentially with peptide length, a library of even relatively short oligopeptides may comprise sufficient diversity to statistically ensure unique peptide functionalization over a particle surface. For example, a peptide comprising of ten amino acids selected from a set of 20 proteinogenic amino acids may comprise more than 10¹³ different sequences, so that a particle comprising 10⁶ random sequence peptides of length 10 has less than a 10⁻²% chance of containing two identical peptides.

Peptide surface functionalizations may be distributed over a particle surface in a random or an ordered fashion. In some embodiments, a plurality of peptide surface functionalizations on a single particle may be spatially separated, such that a first region of the particle comprises a first peptide surface functionalization and a second region of the particle comprises a second surface functionalization.

A peptide surface functionalization may comprise a range of peptide masses or lengths. In some embodiments, a peptide surface functionalization is an amino acid dimer surface functionalization, an amino acid trimer surface functionalization, an oligopeptide surface functionalization (e.g., comprising a length between about 2 and about 30 amino acids), a polypeptide surface functionalization (e.g., comprising a length of greater than about 30 amino acids), or a protein surface functionalization (e.g., a peptide comprising a defined structure). A plurality of peptide surface functionalizations may comprise peptides of identical lengths. A plurality of peptide surface functionalizations comprise peptides of different lengths. A plurality of peptide surface functionalizations may be a plurality of oligopeptide surface functionalizations. A plurality of peptide surface functionalizations may comprise peptides of between 5 and 12 amino acids in length. A plurality of peptide surface functionalizations may comprise peptides of between 4 and 8, between 4 and 10, between 4 and 12, between 4 and 15, between 4 and 20, between 4 and 25, between 5 and 8, between 5 and 10, between 5 and 12, between 5 and 15, between 5 and 20, between 5 and 25, between 6 and 8, between 6 and 10, between 6 and 12, between 6 and 15, between 6 and 20, between 6 and 25, between 7 and 10, between 7 and 12, between 7 and 15, between 7 and 20, between 7 and 25, between 8 and 10, between 8 and 12, between 8 and 15, between 8 and 20, between 8 and 25, between 10 and 12, between 10 and 15, between 10 and 20, between 10 and 25, between 12 and 15, between 12 and 20, between 12 and 25, between 15 and 20, between 15 and 25, between 20 and 25, or between 20 and 30 amino acids in length.

A peptide surface functionalization may comprise a subset of amino acid types. As different amino acid types confer different properties to peptides, the properties of a peptide surface functionalization may be at least partially determined by constructing the peptide surface functionalization from a limited number of amino acid types. For example, glutamic acid and aspartic acid tend to lower the isoelectric point of peptides to which they are attached, while histidine, lysine, and arginine tend to raise peptide isoelectric points while simultaneously providing nucleophilic character. Peptide surface functionalization properties, and thus particle physicochemical surface properties, can be regulated by controlling the types and ratios between types of constituent amino acids. In many cases, peptide surface functionalizations will lack cysteine residues to prevent thiol nucleophilic behavior, redox activity, and crosslinking. In many cases, peptide surface functionalizations will comprise a defined ratio or range of ratios of acidic and basic sidechains as a means for controlling isoelectric point and charge. A peptide surface functionalization may be free of at least one of cysteine, methionine, tryptophan, tyrosine, phenylalanine, and derivatives thereof. A peptide functionalization may be free of at least two of cysteine, methionine, tryptophan, tyrosine, phenylalanine, and derivatives thereof. A peptide functionalization may be free of at least three of cysteine, methionine, tryptophan, tyrosine, phenylalanine, and derivatives thereof. A peptide functionalization may be free of at least four of cysteine, methionine, tryptophan, tyrosine, phenylalanine, and derivatives thereof. A peptide functionalization may be free of cysteine, methionine, tryptophan, tyrosine, phenylalanine, and derivatives thereof. A peptide may comprise amino acids selected from the group consisting of alanine, arginine, aspartic acid, glutamic acid, glutamine, glycine, histidine, isoleucine, leucine, lysine, proline, serine, asparagine, threonine, valine, and derivatives thereof. A peptide may comprise amino acids selected from the group consisting of alanine, arginine, aspartic acid, cysteine, glutamic acid, glutamine, glycine, histidine, isoleucine, leucine, lysine, methionine, phenylalanine, proline, serine, asparagine, threonine, tryptophan, tyrosine, valine, and derivatives thereof. A plurality of peptides may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, or at most 20 types of amino acids. A peptide may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, or at most 20 types of amino acids. A peptide may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 types of amino acids. A plurality of peptides may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 types of amino acids.

A peptide functionalization may comprise a non-proteinogenic amino acid. The non-proteinogenic amino acid may be a chemically modified form of a proteinogenic amino acid. Such a chemical modification may include acylation, alkylation, amidation, deamidation, carbamylation, carbonylation, carboxylation, decarboxylation, citrullination, flavination, glycosylation, halogenation, hydroxylation, nitrosylation, oxidation, phosphorylation, prenylation, racemization, reduction, succinylation, sulfation, or any combination thereof. The non-proteinogenic amino acid may comprise a post-translational modification.

A peptide functionalization may provide a specific isoelectric point to a particle. A particle may comprise an isoelectric point within 2, within 1.5, within 1, within 0.75, within 0.5, within 0.25, or within 0.1 pH units of a peptide functionalization coupled to its surface. A method may comprise forming a biomolecule corona at a pH in which a particle and an analyte comprise opposite charges, or in which the particle and analyte are both neutral. A method may comprise separating a plurality of particles by their isoelectric points (e.g., by isoelectric focusing). A peptide functionalization may comprise an isoelectric point between 4 and 10. A peptide functionalization may comprise an isoelectric point between 7 and 10. A peptide functionalization may comprise an isoelectric point between 4 and 7. A peptide functionalization may comprise an isoelectric point between 5 and 10. A peptide functionalization may comprise an isoelectric point between 6 and 8. A particle may comprise a plurality of peptide functionalizations with different isoelectric points. A peptide functionalization or a plurality of peptide functionalizations may provide a plurality of pka values to a particle.

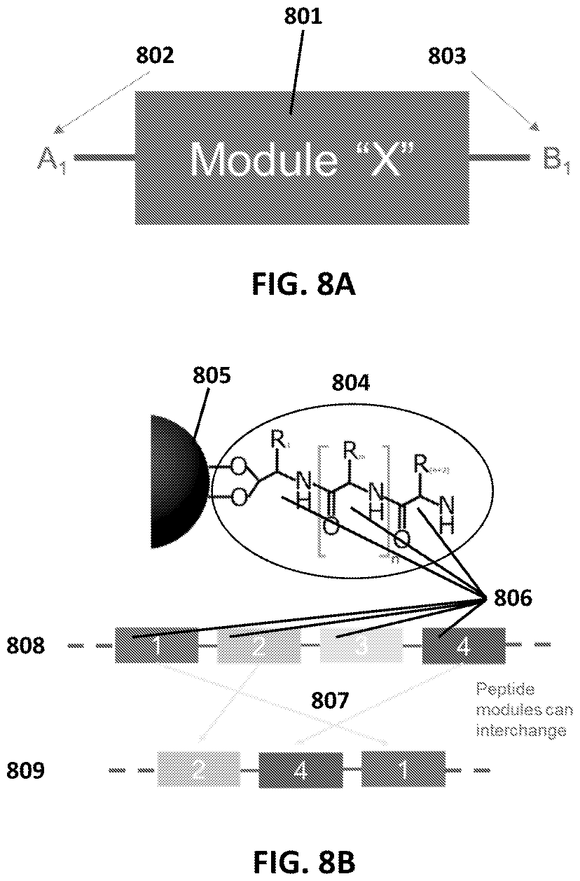

A peptide functionalization or a library of peptide functionalizations may be constructed from modular units. The modular units may comprise individual amino acids, oligomeric units, such as oligopeptides comprising between 2 and 6 amino acid units, or non-peptidic chemical or material units, such as succinyl linkers. For example, a plurality of modular units may comprise oligopeptides having about 1 to about 6 amino acids. A modular unit may be biodegradable (e.g., configured for metabolization and mineralization by a biological system). A diagram of a modular unit structure consistent with the present disclosure is provided in FIG. 8A. A modular unit may comprise an amino acid, an oligopeptide, a non-peptide moiety (e.g., a nucleotide), or any combination thereof 801. A modular unit may be linear or branched. A modular unit may comprise a first reactive handle 802 and a second reactive handle 803. Each reactive handle may comprise a different coupling specificity. For example, a first reactive handle may be configured to only couple with a third type of reactive handle, while a second reactive handle may be configured to couple only with a fourth type of reactive handle. Conversely, two reactive handles may share a specificity. For example, a first reactive handle may be configured to couple with a third type or a fourth type of reactive handle, while a second reactive handle may be configured to couple with the fourth type of reactive handle and a fifth type of reactive handle. In other cases, a first reactive handle is identical to a second reactive handle.

A modular unit may be individually addressable from among a plurality of modular units (e.g., may comprise a unique chemical reactivity or physical property from among the modular units of a peptide). Two or more modular units may be connected by a linking element. Such a linking element may be non-peptidic, and for example may comprise a saccharide, lipid, nucleic acid, or alkyl backbone for incorporation into a peptide functionalization. Separate modular units may be configured to undergo positional exchange, such that a first modular unit from a first peptide functionalization exchanges positions with a second modular unit from the first peptide functionalization or from a second peptide functionalization. Furthermore, an individual modular unit may be configured for removal, modification (e.g., functional group coupling, oxidation, or reduction), or substitution by a separate modular unit. Modifiable and substitutable modular unit building blocks may be used to rapidly generate diverse peptide functionalization libraries. Such a peptide functionalization may comprise between 7 and 20 amino acid residues.

An example of such a modular peptide binding molecule is provided in FIG. 8B. The peptide binding molecule 804 may be coupled to a particle 805. The peptide binding molecule may comprise a plurality of modular units 806. The units may be capable of intermolecular (e.g., with another peptide binding molecule) or intramolecular exchange 807, thereby providing a mechanism for a first peptide binding molecule 808 to generate a second peptide binding molecule 809 comprising a common modular unit.

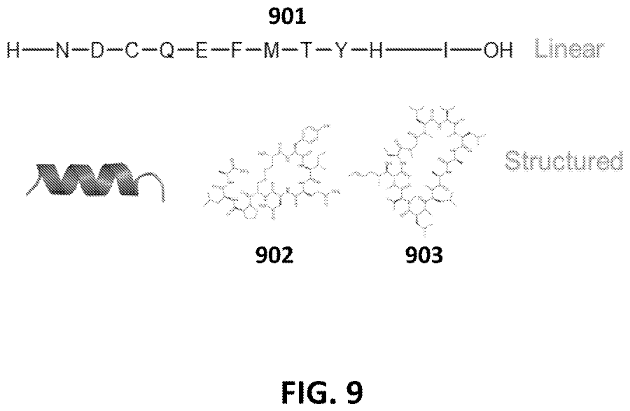

FIG. 9 provides examples of peptide binding molecules with different structural configurations. A peptide binding molecule may comprise a linear structure, for example that of 901. A peptide binding molecule may also comprise a branched or cyclic structure, as is illustrated in 902 and 903.

### Particle Panels

The present disclosure provides compositions and methods of use thereof for assaying a sample for proteins. The present disclosure provides systems and methods for assaying using one or more surface. In some embodiments, a surface may comprise a surface of a high surface-area material, such as nanoparticles, particles, or porous materials. As used herein, a “surface” may refer to a surface for assaying biomolecules. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof. Compositions described herein include particle panels comprising one or more than one distinct particle types. Particle panels described herein can vary in the number of particle types and the diversity of particle types in a single panel. For example, particles in a panel may vary based on size, polydispersity, shape and morphology, surface charge, surface chemistry and functionalization, and base material. Panels may be incubated with a sample to be analyzed for proteins and protein concentrations. Proteins in the sample adsorb to the surface of the different particle types in the particle panel to form a protein corona. The exact protein and the concentration of protein that adsorbs to a certain particle type in the particle panel may depend on the composition, size, and surface charge of said particle type. Thus, each particle type in a panel may have different protein coronas due to adsorbing a different set of proteins, different concentrations of a particular protein, or a combination thereof. Each particle type in a panel may have mutually exclusive protein coronas or may have overlapping protein coronas. Overlapping protein coronas can overlap in protein identity, in protein concentration, or both.

The present disclosure also provides methods for selecting a particle types for inclusion in a panel depending on the sample type. Particle types included in a panel may be a combination of particles that are optimized for removal of highly abundant proteins. Particle types also consistent for inclusion in a panel are those selected for adsorbing particular proteins of interest. The particles can be nanoparticles. The particles can be microparticles. The particles can be a combination of nanoparticles and microparticles.

A particle panel including any number of distinct particle types disclosed herein, enriches and identifies a single protein or protein group. In some embodiments, the single protein or protein group may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some embodiments, the particle panel including any number of distinct particle types disclosed herein, enriches and identifies a single protein or protein group by binding different domains, sequences, or epitopes of the single protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group.

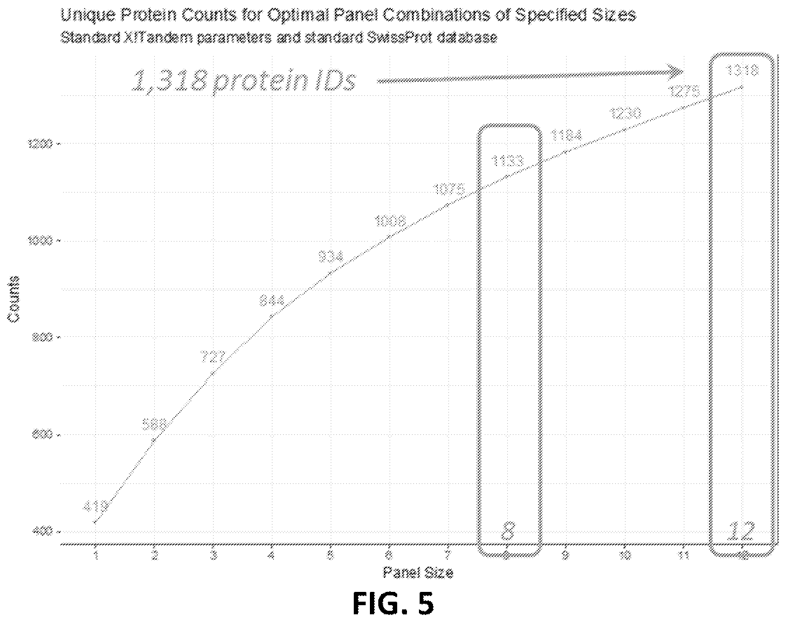

A particle panel can have more than one particle type. Increasing the number of particle types in a panel can be a method for increasing the number of proteins that can be identified in a given sample. An example of how increasing panel size may increase the number of identified proteins is shown in FIG. 5, in which a panel size of one particle type identified 419 different proteins, a panel size of two particle types identified 588 different proteins, a panel size of three particle types identified 727 different proteins, a panel size of four particle types identified 844 proteins, a panel size of five particle types identified 934 different proteins, a panel size of six particle types identified 1008 different proteins, a panel size of seven particle types identified 1075 different proteins, a panel size of eight particle types identified 1133 different proteins, a panel size of nine particle types identified 1184 different proteins, a panel size of 10 particle types identified 1230 different proteins, a panel size of 11 particle types identified 1275 different proteins, and a panel size of 12 particle types identified 1318 different proteins.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

### Non-Specific Binding

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.).

Biomolecules may adsorb onto a surface through non-specific binding on a surface at various densities. In some embodiments, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some embodiments, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some embodiments, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some embodiments, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some embodiments, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some embodiments, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some embodiments, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some embodiments, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some embodiments, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some embodiments, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm².

Adsorbed biomolecules may comprise various types of proteins. In some embodiments, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some embodiments, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins.

In some embodiments, proteins in a biological sample may comprise at least about 1, 2, 3, 4, 5, 6, 7, 8, 9, 10, 15, 20, 25, or 30 orders of magnitudes in concentration. In some embodiments, proteins in a biological sample may comprise at most about 1, 2, 3, 4, 5, 6, 7, 8, 9, 10, 15, 20, 25, or 30 orders of magnitudes in concentration.

### Biomolecule Coronas

The present disclosure provides a variety of compositions, systems, and methods for collecting biomolecules on nanoparticles and microparticles (as well as other types of sensor elements such as polymer matrices, filters, rods, and extended surfaces). A particle may adsorb a plurality of biomolecules upon contact with a biological sample, thereby forming a biomolecule corona on the surfaces of the particles. The biomolecule corona may comprise proteins, lipids, nucleic acids, metabolites, saccharides, small molecules (e.g., sterols), and other biological species present in a sample. A biomolecule corona comprising proteins may also be referred to as a ‘protein corona’, and may refer to all constituents adsorbed to a particle (e.g., proteins, lipids, nucleic acids, and other biomolecules), or may refer only to proteins adsorbed to the particle.

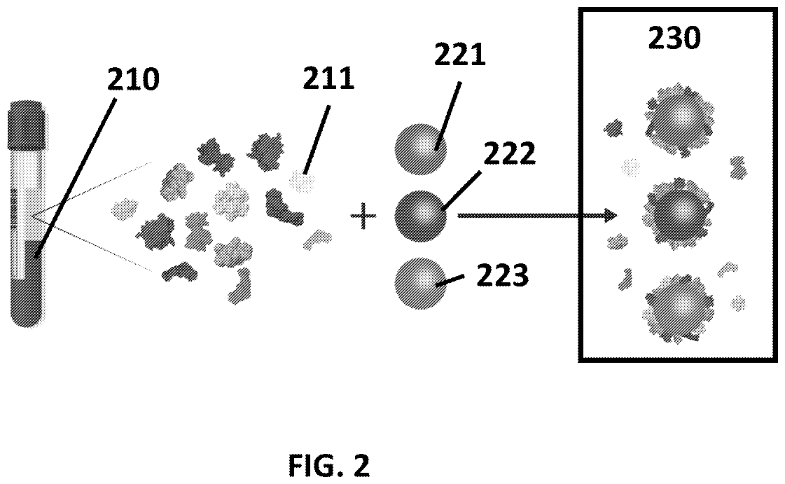

FIG. 2 provides a schematic overview of biomolecule formation, wherein a plurality of particles 221, 222, & 223 particles are contacted with a biological sample 210 comprising biomolecules molecules 211, and wherein each particle adsorbs a plurality of biomolecules from the biological sample to its surface 230. The different particles may be distinct particle types (depicted in the center of the figure, with the top, middle, and bottom spheres representing the three distinct particle types), such that each particle differs from the other particles by at least one physicochemical property. This difference in physicochemical properties can lead to the formation of different protein corona compositions on the particle surfaces.

The composition of the biomolecule corona may depend on a property of the particle. In many cases, the composition of the biomolecule corona is strongly dependent on the surface of the particle. Characteristics such as particle surface material (e.g., ceramic, polymer, metal, metal oxide, graphite, silicon dioxide, etc.), surface texture (rough, smooth, grooved, etc.), surface functionalization (e.g., carboxylate functionalized, amine functionalized, small molecule (e.g., saccharide) functionalized, etc.), shape, curvature, and size can each independently serve as major determinants for biomolecule corona composition. In addition to surface features, the particle core composition, particle density, and particle surface area to mass ratio may each influence biomolecule corona composition. For example, two particles comprising the same surfaces and different cores may form different biomolecule coronas upon contact with the same sample.

Biomolecule corona formation may also be influenced by sample composition. For example, a first sample condition (e.g., low salinity) might favor the solubility of a particular analyte (e.g., an isoform of Bone Morphogenic Protein 1 (BMP1)), and thereby disfavor its binding in a biomolecule corona, while a second sample condition (e.g., high salinity) may diminish the solubility of the analyte, thereby driving its incorporation into a biomolecule corona.

Biomolecule corona composition may also depend on molecular level interactions between the biomolecules, themselves. An energetically favorable interaction between two biomolecules may promote their co-incorporation into a biomolecule corona. For example, if a first protein adsorbed to a particle comprises an affinity for a second protein in solution, the first protein may bind to a portion of the second protein, thereby driving its binding to the particle or to other proteins of the biomolecule corona of the particle. Analogously, a first biomolecule disposed within a biomolecule corona may comprise an energetically unfavorable interaction with a second biomolecule in a biological sample, thereby disfavoring its incorporation into a biomolecule corona. In part owing to these inter-biomolecule dependencies, biomolecule coronas provide sensitive platforms for directly and indirectly sensing biomolecules from a biological sample.

### Protein Analysis Methods

In some embodiments, a method of the present disclosure may comprise using a composition improving assay. In some embodiments, an untargeted assay may be a composition improving assay. In some embodiments, a composition improving assay may improve access to a subset of biomolecules in a biological sample. In some embodiments, a composition improving assay may improve detection to a subset of biomolecules in a biological sample. In some embodiments, a composition improving assay may improve identification to a subset of biomolecules in a biological sample. In some embodiments, the subset of biomolecules may be low-abundance biomolecules. In some embodiments, the subset of biomolecules may be rare biomolecules. In some embodiments, a dynamic range of a biological sample may be compressed using a composition improving assay. In some embodiments, a dynamic range may be compressed by at least about 1, 2, 3, 4, 5, 6, 7, 8, 9, 10, 11, 12, 13, 14, or 15 magnitudes.

In some embodiments, the composition improving assay may comprise providing one or more of surface regions comprising one or more surface types. In some embodiments, the composition improving assay may comprise contacting the biological sample with the one or more surface regions to yield a set of adsorbed biomolecules on the one or more surface regions. In some embodiments, the composition improving assay may comprise desorbing, from the one or more surface regions, at least a portion of the set of adsorbed biomolecules to yield the set of proteins. In some embodiments, the composition improving assay may comprise contacting the biological sample with the one or more surface regions to capture a set of biomolecules on the one or more surface regions. In some embodiments, the composition improving assay may comprise releasing, from the one or more surface regions, at least a portion of the set of biomolecules to yield the set of proteins. In some embodiments, the one or more surface regions are disposed on a single continuous surface. In some embodiments, the one or more surface regions are disposed on one or more discrete surfaces. In some embodiments, the one or more discrete surfaces are surfaces of one or more particles. In some embodiments, the one or more particles may comprise a nanoparticle. In some embodiments, the one or more particles may comprise a microparticle. In some embodiments, the one or more particles may comprise a porous particle. In some embodiments, the one or more particles may comprise a bifunctional, trifunctional, or N-functional particle.

In some embodiments, the composition improving assay may comprise providing a plurality of surface regions comprising a plurality of surface types. In some embodiments, the composition improving assay may comprise contacting the biological sample with the plurality of surface regions to yield a set of adsorbed biomolecules on the plurality of surface regions. In some embodiments, the composition improving assay may comprise desorbing, from the plurality of surface regions, at least a portion of the set of adsorbed biomolecules to yield the set of proteins. In some embodiments, the composition improving assay may comprise contacting the biological sample with the plurality of surface regions to capture a set of biomolecules on the plurality of surface regions. In some embodiments, the composition improving assay may comprise releasing, from the plurality of surface regions, at least a portion of the set of biomolecules to yield the set of proteins. In some embodiments, the plurality of surface regions is disposed on a single continuous surface. In some embodiments, the plurality of surface regions is disposed on a plurality of discrete surfaces. In some embodiments, the plurality of discrete surfaces are surfaces of a plurality of particles. In some embodiments, the plurality of particles may comprise a nanoparticle. In some embodiments, the plurality of particles may comprise a microparticle. In some embodiments, the plurality of particles may comprise a porous particle. In some embodiments, the plurality of particles may comprise a bifunctional, trifunctional, or N-functional particle.

The particles and methods of use thereof disclosed herein may bind a large number of different biomolecules (e.g., proteins) in a biological sample (e.g., a biofluid). For example, a particle disclosed herein may be incubated with a biological sample to form a protein corona comprising at least 5 different proteins, at least 10 different proteins, at least 15 different proteins, at least 20 different proteins, at least 25 different proteins, at least 30 different proteins, at least 40 different proteins, at least 50 different proteins, at least 60 different proteins, at least 80 different proteins, 100 different proteins, at least 120 different proteins, at least 140 different proteins, at least 160 different proteins, at least 180 different proteins, at least 200 different proteins, at least 220 different proteins, at least 240 different proteins, at least 260 different proteins, at least 280 different proteins, at least 300 different proteins, at least 320 different proteins, at least 340 different proteins, at least 360 different proteins, at least 380 different proteins, at least 400 different proteins, at least 420 different proteins, at least 440 different proteins, at least 460 different proteins, at least 480 different proteins, at least 500 different proteins, at least 520 different proteins, at least 540 different proteins, at least 560 different proteins, at least 580 different proteins, at least 600 different proteins, at least 620 different proteins, at least 640 different proteins, at least 660 different proteins, at least 680 different proteins, at least 700 different proteins, at least 720 different proteins, at least 740 different proteins, at least 760 different proteins, at least 780 different proteins, at least 800 different proteins, at least 820 different proteins, at least 840 different proteins, at least 860 different proteins, at least 880 different proteins, at least 900 different proteins, at least 920 different proteins, at least 940 different proteins, at least 960 different proteins, at least 980 different proteins, at least 1000 different proteins, at least 1100 different proteins, at least 1200 different proteins, at least 1300 different proteins, at least 1400 different proteins, at least 1500 different proteins, at least 1600 different proteins, at least 1800 different proteins, at least 2000 different proteins, from 100 to 2000 different proteins, from 150 to 1500 different proteins, from 200 to 1200 different proteins, from 250 to 850 different proteins, from 300 to 800 different proteins, from 350 to 750 different proteins, from 400 to 700 different proteins, from 450 to 650 different proteins, from 500 to 600 different proteins, from 200 to 250 different proteins, from 250 to 300 different proteins, from 300 to 350 different proteins, from 350 to 400 different proteins, from 400 to 450 different proteins, from 450 to 500 different proteins, from 500 to 550 different proteins, from 550 to 600 different proteins, from 600 to 650 different proteins, from 650 to 700 different proteins, from 700 to 750 different proteins, from 750 to 800 different proteins, from 800 to 850 different proteins, from 850 to 900 different proteins, from 900 to 950 different proteins, from 950 to 1000 different proteins, or over 1000 different proteins. In some embodiments, several different types of particles may be used, separately or in combination, to identify large numbers of proteins in a particular biological sample. In other words, particles may be multiplexed in order to bind and identify large numbers of proteins in a biological sample. Protein corona analysis may compress the dynamic range of the analysis compared to a protein analysis of the original sample.

The particle panels disclosed herein may be used to identify the number of distinct proteins disclosed herein, and/or any of the specific proteins disclosed herein, over a wide dynamic range. As used herein, a dynamic range may denote a log 10 value of a ratio of the highest and lowest abundance species of a specified type. Enriching or assaying species over a dynamic range may refer to the abundances of those species in the sample from which they were assayed or derived. For example, the particle panels disclosed herein comprising distinct particle types, may enrich for proteins in a sample, which can be identified using the Proteograph workflow, over the entire dynamic range at which proteins are present in a sample (e.g., a plasma sample). In some embodiments, a particle panel including any number of distinct particle types disclosed herein, may enrich and identify proteins over a dynamic range of at least 2. In some embodiments, a particle panel including any number of distinct particle types disclosed herein, may enrich and identify proteins over a dynamic range of at least 3. In some embodiments, a particle panel including any number of distinct particle types disclosed herein, may enrich and identify proteins over a dynamic range of at least 4. In some embodiments, a particle panel including any number of distinct particle types disclosed herein, may enrich and identify proteins over a dynamic range of at least 5. In some embodiments, a particle panel including any number of distinct particle types disclosed herein, may enrich and identify proteins over a dynamic range of at least 6. In some embodiments, a particle panel including any number of distinct particle types disclosed herein, may enrich and identify proteins over a dynamic range of at least 7. In some embodiments, a particle panel including any number of distinct particle types disclosed herein, may enrich and identify proteins over a dynamic range of at least 8. In some embodiments, a particle panel including any number of distinct particle types disclosed herein, may enrich and identify proteins over a dynamic range of at least 9. In some embodiments, a particle panel including any number of distinct particle types disclosed herein, may enrich and identify proteins over a dynamic range of at least 10. In some embodiments, a particle panel including any number of distinct particle types disclosed herein, may enrich and identify proteins over a dynamic range of at least 11. In some embodiments, a particle panel including any number of distinct particle types disclosed herein, may enrich and identify proteins over a dynamic range of at least 12. In some embodiments, a particle panel including any number of distinct particle types disclosed herein, may enrich and identify proteins over a dynamic range of at least 13. In some embodiments, a particle panel including any number of distinct particle types disclosed herein, may enrich and identify proteins over a dynamic range of at least 14. In some embodiments, a particle panel including any number of distinct particle types disclosed herein, may enrich and identify proteins over a dynamic range of at least 15. In some embodiments, a particle panel including any number of distinct particle types disclosed herein, may enrich and identify proteins over a dynamic range of at least 20. In some embodiments, a particle panel including any number of distinct particle types disclosed herein, may enrich and identify proteins over a dynamic range of from 2 to 100. In some embodiments, a particle panel including any number of distinct particle types disclosed herein, may enrich and identify proteins over a dynamic range of from 2 to 20. In some embodiments, a particle panel including any number of distinct particle types disclosed herein, may enrich and identify proteins over a dynamic range of from 2 to 10. In some embodiments, a particle panel including any number of distinct particle types disclosed herein, may enrich and identify proteins over a dynamic range of from 2 to 5. In some embodiments, a particle panel including any number of distinct particle types disclosed herein may enrich and identify proteins over a dynamic range of from 5 to 10.

The numbers and types of biomolecules (e.g., proteins) collected in a biomolecule corona may depend on the amount of time a particle is incubated with a sample. In many cases, biomolecule corona formation will comprise a time dependence, such that different sets of biomolecules collect on a particle at different rates. Further complicating this process, a biomolecule may comprise a time-dependent adsorption or desorption profile. For example, a biomolecule may rapidly collect on a particle during a first phase of biomolecule corona formation, and subsequently slowly desorb from the particle as other biomolecules bind. Accordingly, the length of time over which a particle is contacted to a sample can influence the mass and composition of a resulting biomolecule corona. An assay may generate a biomolecule corona in less than 2 hours. An assay may generate a biomolecule corona in less than 1.5 hours. An assay may generate a biomolecule corona in less than 1 hour. An assay may generate a biomolecule corona in less than 30 minutes. An assay may generate a biomolecule corona in less than 20 minutes. An assay may generate a biomolecule corona in less than 15 minutes. An assay may generate a biomolecule corona in less than 12 minutes. An assay may generate a biomolecule corona in less than 10 minutes. An assay may comprise incubating a particle with a sample for at least 10 minutes to generate a biomolecule corona. An assay may comprise incubating a particle with a sample for at least 12 minutes to generate a biomolecule corona. An assay may comprise incubating a particle with a sample for at least 15 minutes to generate a biomolecule corona. An assay may comprise incubating a particle with a sample for at least 20 minutes to generate a biomolecule corona. An assay may comprise incubating a particle with a sample for at least 30 minutes to generate a biomolecule corona. An assay may comprise incubating a particle with a sample for at least 45 minutes to generate a biomolecule corona. An assay may comprise incubating a particle with a sample for at least 60 minutes to generate a biomolecule corona. An assay may comprise incubating a particle with a sample for at least 90 minutes to generate a biomolecule corona. An assay may comprise incubating a particle with a sample for at least 120 minutes to generate a biomolecule corona. A biomolecule corona may comprise at least 10⁻¹¹ mg of biomolecules per square millimeter (mm²) of particle surface area. A biomolecule corona may comprise at least 5×10⁻¹¹ mg of biomolecules per square millimeter (mm²) of particle surface area. A biomolecule corona may comprise at least 10⁻¹⁰ mg of biomolecules per square millimeter (mm²) of particle surface area. A biomolecule corona may comprise at least 5×10⁻¹⁰ mg of biomolecules per square millimeter (mm²) of particle surface area. A biomolecule corona may comprise at least 10⁻⁹ mg of biomolecules per square millimeter (mm²) of particle surface area. A biomolecule corona may comprise at least 5×10⁻⁹ mg of biomolecules per square millimeter (mm²) of particle surface area. A biomolecule corona may comprise at least 10⁻⁸ mg of biomolecules per square millimeter (mm²) of particle surface area. A biomolecule corona may comprise at least 5×10⁻⁸ mg of biomolecules per square millimeter (mm²) of particle surface area. A biomolecule corona may comprise at least 10⁻⁷ mg of biomolecules per square millimeter (mm²) of particle surface area. A biomolecule corona may comprise at least 10⁻¹¹ mg of proteins per square millimeter (mm²) of particle surface area. A biomolecule corona may comprise at least 5×10⁻¹¹ mg of proteins per square millimeter (mm²) of particle surface area. A biomolecule corona may comprise at least 10⁻¹⁰ mg of proteins per square millimeter (mm²) of particle surface area. A biomolecule corona may comprise at least 5×10⁻¹⁰ mg of proteins per square millimeter (mm²) of particle surface area. A biomolecule corona may comprise at least 10⁻⁹ mg of proteins per square millimeter (mm²) of particle surface area. A biomolecule corona may comprise at least 5×10⁻⁹ mg of proteins per square millimeter (mm²) of particle surface area. A biomolecule corona may comprise at least 10⁻⁸ mg of proteins per square millimeter (mm²) of particle surface area. A biomolecule corona may comprise at least 5×10⁻⁸ mg of proteins per square millimeter (mm²) of particle surface area. A biomolecule corona may comprise at least 10⁻⁷ mg of proteins per square millimeter (mm²) of particle surface area. A biomolecule corona may comprise an expanded or compressed dynamic range relative to a sample. For example, a biomolecule corona may collect proteins spanning 7 orders of magnitude in concentration in a sample over an abundance range spanning 4 orders of magnitude, thereby compressing the dynamic range of the collected proteins.

Biomolecules collected on a particle may be subjected to further analysis. A method may comprise collecting a biomolecule corona or a subset of biomolecules from a biomolecule corona. The collected biomolecule corona or the collected subset of biomolecules from the biomolecule corona may be subjected to further particle-based analysis (e.g., particle adsorption). The collected biomolecule corona or the collected subset of biomolecules from the biomolecule corona may be purified or fractionated (e.g., by a chromatographic method). The collected biomolecule corona or the collected subset of biomolecules from the biomolecule corona may be analyzed (e.g., by mass spectrometry).

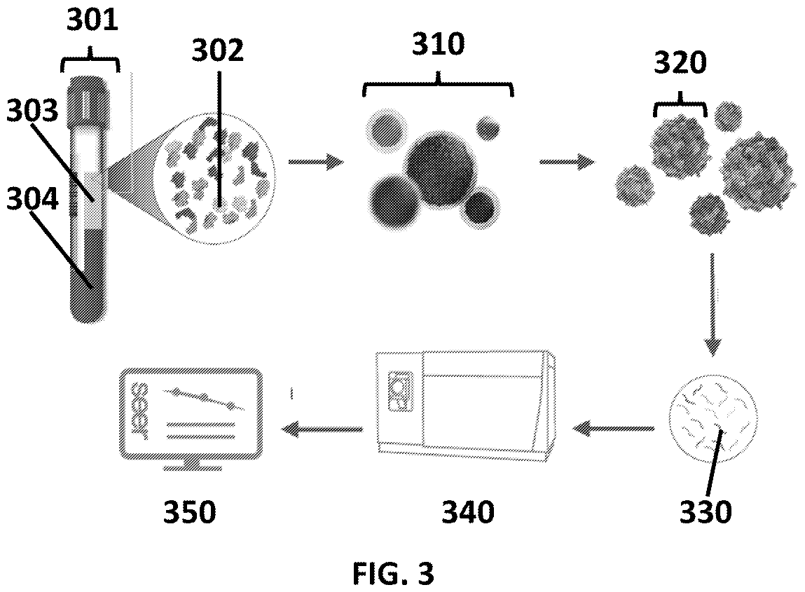

FIG. 3 provides an example of a particle-based biomolecule corona (e.g., protein corona) assay consistent with the present disclosure. A biological sample (e.g., human plasma) 301 comprising a plurality of biomolecules 302 may be contacted to a plurality of particles 310. The sample may be treated, diluted, or split into a plurality of fractions 303 and 304 prior to analysis. For example, a whole blood sample may be fractionated into plasma and erythrocyte portions. Upon contact with the particles, a subset or the entirety of the plurality of biomolecules may adsorb to the particles, thereby forming biomolecule coronas 320 bound to the surfaces of the particles. Unbound biomolecules may be separated from the biomolecule coronas (e.g., through wash steps). The biomolecule coronas, or subsets thereof, may be collected from the particles. Alternatively, biomolecules of the biomolecule coronas may be fragmented or chemically treated while bound to the particles. In some assays, biomolecules (e.g., proteins) are fragmented (e.g., digested) while disposed in the biomolecule coronas to yield biomolecule (e.g., peptide) fragments 330. Biomolecules (or their chemically treated or fragmented derivatives) may be analyzed 340, for example by mass spectrometry, to yield data 350 representative of biomolecules 302 from the biological sample 301. The data may be analyzed to identify a biological state of the biological sample.

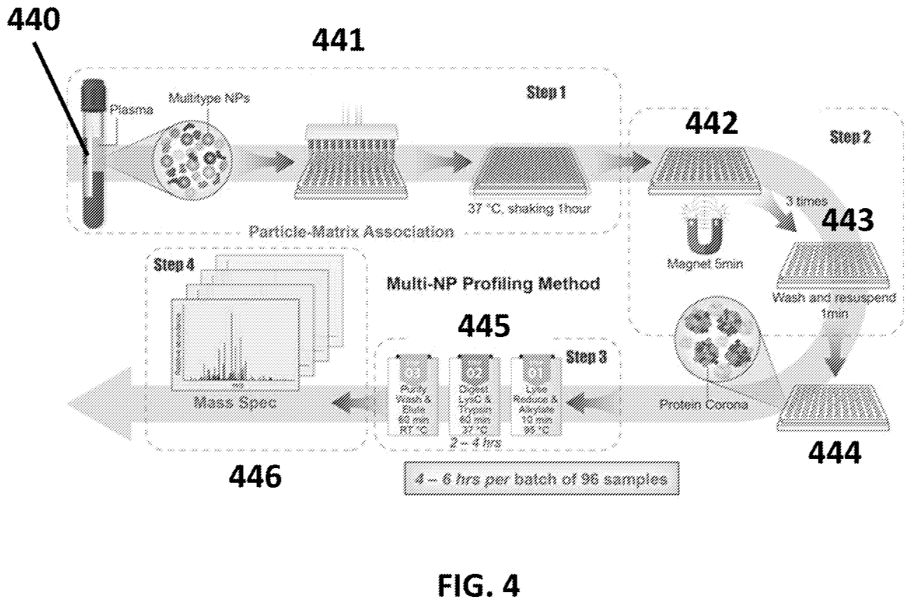

FIG. 4 illustrates an example of a biomolecule corona (e.g., protein corona) analysis workflow consistent with the present disclosure which includes: particle incubation with a biological sample 440 (e.g., plasma), thereby adsorbing biomolecules from the plasma sample to the particles to form biomolecule coronas; partitioning 441 of the particle-plasma sample mixture into a plurality of wells on a 96 well plate; particle collection 442 (e.g., with a magnet); a wash step or plurality of wash steps 443 to remove analytes not adsorbed to the particles; 444 resuspension of the particles and the biomolecules adsorbed thereto; optionally, biomolecule corona digestion or chemical treatment 445 (e.g., protein reduction and digestion); and analysis of the biomolecule coronas or of biomolecules derived therefrom 446 (e.g., by liquid chromatography-mass spectrometry (LC-MS) analysis). While this example provides parallel analyses across 96 well plate wells, a method may comprise a single sample volume or a plurality of sample volumes ranging from two to hundreds of thousands of sample volumes. Furthermore, while this example provides contacting a sample with particles prior to partitioning, a method may alternatively comprise partitioning a sample (e.g., into separate wells of a well plate) prior to contacting with particles. In some embodiments, sample may be added to partitions comprising particles. For example, a well plate may be provided with particles, buffer, and reagents in dry form, such that a method of use may comprise adding solution to the wells to resuspend the particles and dissolve the buffer and reagents, and then adding sample to the wells.

Protein corona analysis may comprise an automated component. For example, an automated instrument may contact a sample with a particle or particle panel, identify proteins on the particle or particle panel (e.g., digest the proteins on the particle or particle panel and perform mass spectrometric analysis), and generate data for identifying a specific biomolecule or a biological state of a sample. The automated instrument may divide a sample into a plurality of volumes, and perform analysis on each volume. The automated instrument may analyze multiple separate samples, for example by disposing multiple samples within multiple wells in a well plate, and performing parallel analysis on each sample.

The particle panels disclosed herein may be used to identifying a number of proteins, peptides, protein groups, or protein classes using a protein analysis workflow described herein (e.g., a protein corona analysis workflow). Protein corona analysis may comprise contacting a sample to distinct particle types (e.g., a particle panel), forming biomolecule corona on the distinct particle types, and identifying the biomolecules in the biomolecule corona (e.g., by mass spectrometry). Feature intensities, as disclosed herein, refers to the intensity of a discrete spike (“feature”) seen on a plot of mass to charge ratio versus intensity from a mass spectrometry run of a sample. These features can correspond to variably ionized fragments of peptides and/or proteins. Using the data analysis methods described herein, feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence).

The methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. Thus, the methods disclosed herein provide for isolating or separating a particle type from unbound protein in a sample. A particle type may be separated by a variety of means, including but not limited to magnetic separation, centrifugation, filtration, or gravitational separation. Particle panels may be incubated with a plurality of spatially isolated samples, wherein each spatially isolated sample is in a well in a well plate (e.g., a 96-well plate). After incubation, the particle types in each of the wells of the well plate can be separated from unbound protein present in the spatially isolated samples by placing the entire plate on a magnet. This simultaneously pulls down the superparamagnetic particles in the particle panel. The supernatant in each sample can be removed to remove the unbound protein. These steps (incubate, pull down) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated samples at the same time.

The methods and compositions of the present disclosure provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A high abundance protein may be present in a sample at concentrations at or above about 1 μM. A high abundance protein may constitute at least 1%, at least 0.1%, or at least 0.05% of the protein mass of a sample. A protein of moderate abundance may be present in a sample at concentrations between about 10 ng/ml and about 10 μg/mL. Examples of proteins that are highly abundant in human plasma include albumin, IgG, and the top 14 proteins in abundance that contribute 95% of the analyte mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 September; 14(9):2375-93. doi: 10.1074/mcp.M114.046813. Epub 2015 Feb. 27.), Farr et al. (J Proteome Res. 2014 Jan. 3; 13(1):60-75. doi: 10.1021/pr4010037. Epub 2013 Dec. 6.), or Pernemalm et al. (Expert Rev Proteomics. 2014 August; 11(4):431-48. doi: 10.1586/14789450.2014.901157. Epub 2014 Mar. 24.).

The methods and compositions disclosed herein may also elucidate protein classes or interactions of the protein classes. A protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); or proteins bearing a post-translational modification (e.g., ubiquitinated or citrullinated proteins). A protein class may contain at least 2 proteins, 5 proteins, 10 proteins, 20 proteins, 40 proteins, 60 proteins, 80 proteins, 100 proteins, 150 proteins, 200 proteins, or more.

The proteomic data of the biological sample can be identified, measured, and quantified using a number of different analytical techniques. For example, proteomic data can be generated using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in EP3548652, WO2019083856, WO2019133892, each of which is incorporated herein by reference in its entirety, and other protein separation techniques.

An assay may comprise protein collection of particles, protein digestion, and mass spectrometric analysis (e.g., MS, LC-MS, LC-MS/MS). The digestion may comprise chemical digestion, such as by cyanogen bromide or 2-Nitro-5-thiocyanatobenzoic acid (NTCB). The digestion may comprise enzymatic digestion, such as by trypsin or pepsin. The digestion may comprise enzymatic digestion by a plurality of proteases. The digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. The digestion may cleave peptides at random positions. The digestion may cleave peptides at a specific position (e.g., at methionines or lysines) or sequence (e.g., glutamate-histidine-glutamate). The digestion may enable similar proteins to be distinguished. For example, an assay may resolve 8 distinct proteins as a single protein group with a first digestion method, and as 8 separate proteins with distinct signals with a second digestion method. The digestion may generate an average peptide fragment length of 8 to 15 amino acids. The digestion may generate an average peptide fragment length of 12 to 18 amino acids. The digestion may generate an average peptide fragment length of 15 to 25 amino acids. The digestion may generate an average peptide fragment length of 20 to 30 amino acids. The digestion may generate an average peptide fragment length of 30 to 50 amino acids.

An assay may rapidly generate and analyze proteomic data. Beginning with an input biological sample (e.g., a buccal or nasal smear, plasma, or tissue), an assay of the present disclosure may generate and analyze proteomic data in less than 7 hours. Beginning with an input biological sample, an assay of the present disclosure may generate and analyze proteomic data in 5-7 hours. Beginning with an input biological sample, an assay of the present disclosure may generate and analyze proteomic data in less than 5 hours. Beginning with an input biological sample, an assay of the present disclosure may generate and analyze proteomic data in 3-5 hours. Beginning with an input biological sample, an assay of the present disclosure may generate and analyze proteomic data in 2-4 hours. Beginning with an input biological sample, an assay of the present disclosure may generate and analyze proteomic data in 2-3 hours. Beginning with an input biological sample, an assay of the present disclosure may generate and analyze proteomic data in less than 3 hours. Beginning with an input biological sample, an assay of the present disclosure may generate and analyze proteomic data in less than 2 hours. The analyzing may comprise identifying a protein group. The analyzing may comprise identifying a protein class. The analyzing may comprise quantifying an abundance of a biomolecule, a peptide, a protein, protein group, or a protein class. The analyzing may comprise identifying a ratio of abundances of two biomolecules, peptides, proteins, protein groups, or protein classes. The analyzing may comprise identifying a biological state.

### Dynamic Range

The biomolecule corona analysis methods described herein may comprise assaying biomolecules in a sample of the present disclosure across a wide dynamic range. The dynamic range of biomolecules assayed in a sample may be a range of measured signals of biomolecule abundances as measured by an assay method (e.g., mass spectrometry, chromatography, gel electrophoresis, spectroscopy, or immunoassays) for the biomolecules contained within a sample. For example, an assay capable of detecting proteins across a wide dynamic range may be capable of detecting proteins of very low abundance to proteins of very high abundance. The dynamic range of an assay may be directly related to the slope of assay signal intensity as a function of biomolecule abundance. For example, an assay with a low dynamic range may have a low (but positive) slope of the assay signal intensity as a function of biomolecule abundance, e.g., the ratio of the signal detected for a high abundance biomolecule to the ratio of the signal detected for a low abundance biomolecule may be lower for an assay with a low dynamic range than an assay with a high dynamic range. In specific cases, dynamic range may refer to the dynamic range of proteins within a sample or assaying method.

The biomolecule corona analysis methods described herein may compress the dynamic range of an assay. The dynamic range of an assay may be compressed relative to another assay if the slope of the assay signal intensity as a function of biomolecule abundance is lower than that of the other assay. For example, a plasma sample assayed using protein corona analysis with mass spectrometry may have a compressed dynamic range compared to a plasma sample assayed using mass spectrometry alone, directly on the sample or compared to provided abundance values for plasma proteins in databases (e.g., the database provided in Keshishian et al., Mol. Cell Proteomics 14, 2375-2393 (2015), also referred to herein as the “Carr database”). The compressed dynamic range may enable the detection of more low abundance biomolecules in a biological sample using biomolecule corona analysis with mass spectrometry than using mass spectrometry alone.

In some embodiments, the dynamic range of a proteomic analysis assay may be the ratio of the signal produced by highest abundance proteins (e.g., the highest 10% of proteins by abundance) to the signal produced by the lowest abundance proteins (e.g., the lowest 10% of proteins by abundance). Compressing the dynamic range of a proteomic analysis may comprise decreasing the ratio of the signal produced by the highest abundance proteins to the signal produced by the lowest abundance proteins for a first proteomic analysis assay relative to that of a second proteomic analysis assay. The protein corona analysis assays disclosed herein may compress the dynamic range relative to the dynamic range of a total protein analysis method (e.g., mass spectrometry, gel electrophoresis, or liquid chromatography).

Provided herein are several methods for compressing the dynamic range of a biomolecular analysis assay to facilitate the detection of low abundance biomolecules relative to high abundance biomolecules. For example, a particle type of the present disclosure can be used to serially interrogate a sample. Upon incubation of the particle type in the sample, a biomolecule corona comprising forms on the surface of the particle type. If biomolecules are directly detected in the sample without the use of said particle types, for example by direct mass spectrometric analysis of the sample, the dynamic range may span a wider range of concentrations, or more orders of magnitude, than if the biomolecules are directed on the surface of the particle type. Thus, using the particle types disclosed herein may be used to compress the dynamic range of biomolecules in a sample. Without being limited by theory, this effect may be observed due to more capture of higher affinity, lower abundance biomolecules in the biomolecule corona of the particle type and less capture of lower affinity, higher abundance biomolecules in the biomolecule corona of the particle type.

A dynamic range of a proteomic analysis assay may be the slope of a plot of a protein signal measured by the proteomic analysis assay as a function of total abundance of the protein in the sample. Compressing the dynamic range may comprise decreasing the slope of the plot of a protein signal measured by a proteomic analysis assay as a function of total abundance of the protein in the sample relative to the slope of the plot of a protein signal measured by a second proteomic analysis assay as a function of total abundance of the protein in the sample. The protein corona analysis assays disclosed herein may compress the dynamic range relative to the dynamic range of a total protein analysis method (e.g., mass spectrometry, gel electrophoresis, or liquid chromatography).

### Kits

Provided herein are kits comprising compositions of the present disclosure that may be used to perform the methods of the present disclosure. A kit may comprise one or more particle types to interrogate a sample to identify a biological state of a sample. In some embodiments, a kit may comprise a particle type provided in TABLE 1. A kit may comprise a reagent for functionalizing a particle (e.g., a reagent for tethering a small molecule functionalization to a particle surface). The kit may be pre-packaged in discrete aliquots. In some embodiments, the kit can comprise a plurality of different particle types that can be used to interrogate a sample. The plurality of particle types can be pre-packaged where each particle type of the plurality is packaged separately. Alternately, the plurality of particle types can be packaged together to contain combination of particle types in a single package. A particle may be provided in dried (e.g., lyophilized) form, or may be provided in a suspension or solution. The particles may be provided in a well plate. For example, a kit may contain an 8 well plate, an 8-384 well plate with particles provided (e.g., sealed) within the wells. For example, a well plate may comprise at least 8, at least 16, at least 24, at least 32, at least 40, at least 48, at least 56, at least 64, at least 72, at least 80, at least 88, at least 96, at least 10⁴, at least 112, at least 120, at least 128, at least 136, at least 144, at least 152, at least 160, at least 168, at least 176, at least 184, at least 192, at least 200, at least 208, at least 216, at least 224, at least 232, at least 240, at least 248, at least 256, at least 264, at least 272, at least 280, at least 288, at least 296, at least 304, at least 312, at least 320, at least 328, at least 336, at least 344, at least 352, at least 360, at least 368, at least 376, at least 384, at least 392, at least 400 wells comprising particles. Two wells in such a well plate may contain different particles or different concentrations of particles. Two wells may comprise different buffers or chemical conditions. For example, a well plate may be provided with different particles in each row of wells and different buffers in each column of rows. A well may be sealed by a removable covering. For example, a kit may comprise a well plate comprising a plastic slip covering a plurality of wells. A well may be sealed by a pierceable covering. For example, a well may be covered by a septum that a needle can pierce to facilitate sample movement into and out of the well.

A kit may comprise a composition for generating a peptide surface functionalization on a particle. The kit may comprise a reagent for attaching a peptide to the surface of a particle. The reagent may activate a surface functionalization or a portion of a surface of a particle to react with a peptide or a linker. The reagent may activate a peptide to react with a particle, a surface functionalization of a particle, or a linker. For example, the reagent may chemically modify and enhance the electrophilicity of C-terminal residues of peptides to facilitate their coupling to particle-derived amines. The kit may comprise a linker comprising a first moiety capable of coupling to a site on a particle and a second moiety capable of coupling to a site on a peptide. The kit may comprise an affinity binding reagent, such as streptavidin, coupled or configured to couple to a particle or peptide, and a ligand, such as biotin, coupled or configured to couple to a peptide.

The kit may comprise a reagent or composition for generating a plurality of peptides. For example, a kit may comprise a protease for generating oligopeptides from a protein sample, as well as a means for coupling the oligopeptides generated therefrom to a particle. The kit may comprise reagents for de novo peptide synthesis, for example a plurality of α-carboxylate activated (e.g., TMS-derivatized) amino acids for stepwise peptide synthesis.

The kit may comprise a reagent for functionalizing a peptide, such as a peptide coupled to the surface of a particle. The reagent may chemically modify the peptide at a specific residue or moiety (e.g., a reagent may phosphorylate tyrosine residues of particle-bound peptides). The reagent may cleave the peptide in a sequence specific or non-specific manner. The reagent may couple a first peptide to a second peptide.

### Samples

The present disclosure provides a range of samples that can be assayed using the particles and the methods provided herein. A sample may be a biological sample (e.g., a sample derived from a living organism). A sample may comprise a cell or be cell-free. A sample may comprise a biofluid, such as blood, serum, plasma, urine, or cerebrospinal fluid (CSF). Samples consistent with the present disclosure include biological samples from a subject. The subject may be a human or a non-human animal. Said biological samples can contain a plurality of proteins or proteomic data, which may be analyzed after adsorption of proteins to the surface of the various sensor element (e.g., particle) types in a panel and subsequent digestion of protein coronas. Proteomic data can comprise nucleic acids, peptides, or proteins. A biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract.

A wide range of samples are compatible for use within the methods and compositions of the present disclosure. The biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. The biological sample may comprise multiple biological samples (e.g., pooled plasma from multiple subjects, or multiple tissue samples from a single subject). The biological sample may comprise a single type of biofluid or biomaterial from a single source.

The biological sample may be diluted or pre-treated. The biological sample may undergo depletion (e.g., the biological sample comprises serum) prior to or following contact with a particle or plurality of particles. The biological sample may also undergo physical (e.g., homogenization or sonication) or chemical treatment prior to or following contact with a particle or plurality of particles. The biological sample may be diluted prior to or following contact with a particle or plurality of particles. The dilution medium may comprise buffer or salts, or be purified water (e.g., distilled water). Different partitions of a biological sample may undergo different degrees of dilution. A biological sample or a portion thereof may undergo a 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution.

The compositions and methods of the present disclosure can be used to measure, detect, and identify specific proteins from biological samples. Examples of proteins that can be identified and measured include highly abundant proteins, proteins of medium abundance, and low-abundance proteins. For example, a composition or method may identify at least 1, at least 2, at least 3, at least 4, at least 5, at least 6, at least 7, at least 8, at least 10, at least 12, at least 15, at least 18, at least 20, at least 25, at least 30, at least 35, at least 40, or at least 50 human plasma proteins from the group consisting of albumin, immunoglobulin G (IgG), lysozyme, carcino embryonic antigen (CEA), receptor tyrosine-protein kinase erbB-2 (HER-2/neu), bladder tumor antigen, thyroglobulin, alpha-fetoprotein, prostate specific antigen (PSA), mucin 16 (CA125), carbohydrate antigen 19-9 (CA19.9), carcinoma antigen 15-3 (CA15.3), leptin, prolactin, osteopontin, insulin-like growth factor 2 (IGF-II), 4F2 cell-surface antigen heavy chain (CD98), fascin, sPigR, 14-3-3 eta, troponin I, B-type natriuretic peptide, breast cancer type 1 susceptibility protein (BRCA1), c-Myc proto-oncogene protein (c-Myc), interleukin-6 (IL-6), fibrinogen, epidermal growth factor receptor (EGFR), gastrin, PH, granulocyte colony-stimulating factor (G CSF), desmin, enolase 1 (NSE), folice-stimulating hormone (FSH), vascular endothelial growth factor (VEGF), P21, Proliferating cell nuclear antigen (PCNA), calcitonin, pathogenesis-related proteins (PR), luteinizing hormone (LH), somatostatin S100, insulin. alpha-prolactin, adrenocorticotropic hormone (ACTH), B-cell lymphoma 2 (Bcl 2), estrogen receptor alpha (ER alpha), antigen k (Ki-67), tumor protein (p53), cathepsin D, beta catenin, von Willebrand factor (VWF), CD15, k-ras, caspase 3, ENTH domain-containing protein (EPN), CD10, FAS, breast cancer type 2 susceptibility protein (BRCA2), CD30L, CD30, CGA, CRP, prothrombin, CD44, APEX, transferrin, GM-CSF, E-cadherin, interleukin-2 (IL-2), Bax, IFN-gamma, beta-2-MG, tumor necrosis factor alpha (TNF alpha), cluster of differentiation 340, trypsin, cyclin D1, MG B, XBP-1, HG-1, YKL-40, S-gamma, ceruloplasmin, NESP-55, netrin-1, geminin, GADD45A, CDK-6, CCL21, breast cancer metastasis suppressor 1 (BrMS1), 17betaHDI, platelet-derived growth factor receptor A (PDGRFA), P300/CBP-associated factor (Pcaf), chemokine ligand 5 (CCL5), matrix metalloproteinase-3 (MMP3), claudin-4, and claudin-3

### Biological States

The compositions and methods disclosed herein can be used to identify various biological states in a particular biological sample. For example, a biological state can refer to an elevated or low level of a particular protein or a set of proteins. In other examples, a biological state can refer to identification of a disease, such as cancer. The particles and methods of us thereof can be used to distinguish between two biological states. The two biological states may be related diseases states (e.g., two HRAS mutant colon cancers or different stages of a type of a cancer). The two biological states may be different phases of a disease, such as pre-Alzheimer's and mild Alzheimer's. The two biological states may be distinguished with a high degree of accuracy (e.g., the percentage of accurately identified biological states among a population of samples). For example, the compositions and methods of the present disclosure may distinguish two biological states with at least 60% accuracy, at least 70% accuracy, at least 75% accuracy at least 80% accuracy, at least 85% accuracy, at least 90% accuracy, at least 95% accuracy, at least 98% accuracy, or at least 99% accuracy. The two biological states may be distinguished with a high degree of specificity (e.g., the rate at which negative results are correctly identified among a population of samples). For example, the compositions and methods of the present disclosure may distinguish two biological states with at least 60% specificity, at least 70% specificity, at least 75% specificity at least 80% specificity, at least 85% specificity, at least 90% specificity, at least 95% specificity, at least 98% specificity, or at least 99% specificity.

The methods, compositions, and systems described herein can be used to determine a disease state, and/or prognose or diagnose a disease or disorder. The diseases or disorders contemplated include, but are not limited to, for example, cancer, cardiovascular disease, endocrine disease, inflammatory disease, a neurological disease and the like.

The methods, compositions, and systems described herein can be used to determine, prognose, and/or diagnose a cancer disease state. The term “cancer” is meant to encompass any cancer, neoplastic and preneoplastic disease that is characterized by abnormal growth of cells, including tumors and benign growths. Cancer may, for example, be lung cancer, pancreatic cancer, or skin cancer. In many cases, the methods, compositions and systems described herein are not only able to diagnose cancer (e.g. determine if a subject (a) does not have cancer, (b) is in a pre-cancer development stage, (c) is in early stage of cancer, (d) is in a late stage of cancer) but are able to determine the type of cancer.

The methods, compositions, and systems of the present disclosure can additionally be used to detect other cancers, such as acute lymphoblastic leukemia (ALL); acute myeloid leukemia (AML); cancer in adolescents; adrenocortical carcinoma; childhood adrenocortical carcinoma; unusual cancers of childhood; AIDS-related cancers; kaposi sarcoma (soft tissue sarcoma); AIDS-related lymphoma (lymphoma); primary cns lymphoma (lymphoma); anal cancer; appendix cancer—see gastrointestinal carcinoid tumors; astrocytomas, childhood (brain cancer); atypical teratoid/rhabdoid tumor, childhood, central nervous system (brain cancer); basal cell carcinoma of the skin—see skin cancer; bile duct cancer; bladder cancer; childhood bladder cancer; bone cancer (includes ewing sarcoma and osteosarcoma and malignant fibrous histiocytoma); brain tumors; breast cancer; childhood breast cancer; bronchial tumors, childhood; burkitt lymphoma—see non-hodgkin lymphoma; carcinoid tumor (gastrointestinal); childhood carcinoid tumors; carcinoma of unknown primary; childhood carcinoma of unknown primary; cardiac (heart) tumors, childhood; central nervous system; atypical teratoid/rhabdoid tumor, childhood (brain cancer); embryonal tumors, childhood (brain cancer); germ cell tumor, childhood (brain cancer); primary cns lymphoma; cervical cancer; childhood cervical cancer; childhood cancers; cancers of childhood, unusual; cholangiocarcinoma—see bile duct cancer; chordoma, childhood; chronic lymphocytic leukemia (CLL); chronic myelogenous leukemia (CML); chronic myeloproliferative neoplasms; colorectal cancer; childhood colorectal cancer; craniopharyngioma, childhood (brain cancer); cutaneous t-cell lymphoma—see lymphoma (mycosis fungoides and sézary syndrome); ductal carcinoma in situ (DCIS)—see breast cancer; embryonal tumors, central nervous system, childhood (brain cancer); endometrial cancer (uterine cancer); ependymoma, childhood (brain cancer); esophageal cancer; childhood esophageal cancer; esthesioneuroblastoma (head and neck cancer); ewing sarcoma (bone cancer); extracranial germ cell tumor, childhood; extragonadal germ cell tumor; eye cancer; childhood intraocular melanoma; intraocular melanoma; retinoblastoma; fallopian tube cancer; fibrous histiocytoma of bone, malignant, and osteosarcoma; gallbladder cancer; gastric (stomach) cancer; childhood gastric (stomach) cancer; gastrointestinal carcinoid tumor; gastrointestinal stromal tumors (GIST) (soft tissue sarcoma); childhood gastrointestinal stromal tumors; germ cell tumors; childhood central nervous system germ cell tumors (brain cancer); childhood extracranial germ cell tumors; extragonadal germ cell tumors; ovarian germ cell tumors; testicular cancer; gestational trophoblastic disease; hairy cell leukemia; head and neck cancer; heart tumors, childhood; hepatocellular (liver) cancer; histiocytosis, langerhans cell; hodgkin lymphoma; hypopharyngeal cancer (head and neck cancer); intraocular melanoma; childhood intraocular melanoma; islet cell tumors, pancreatic neuroendocrine tumors; kaposi sarcoma (soft tissue sarcoma); kidney (renal cell) cancer; langerhans cell histiocytosis; laryngeal cancer (head and neck cancer); leukemia; lip and oral cavity cancer (head and neck cancer); liver cancer; lung cancer (non-small cell and small cell); childhood lung cancer; lymphoma; male breast cancer; malignant fibrous histiocytoma of bone and osteosarcoma; melanoma; childhood melanoma; melanoma, intraocular (eye); childhood intraocular melanoma; merkel cell carcinoma (skin cancer); mesothelioma, malignant; childhood mesothelioma; metastatic cancer; metastatic squamous neck cancer with occult primary (head and neck cancer); midline tract carcinoma with nut gene changes; mouth cancer (head and neck cancer); multiple endocrine neoplasia syndromes; multiple myeloma/plasma cell neoplasms; mycosis fungoides (lymphoma); myelodysplastic syndromes, myelodysplastic/myeloproliferative neoplasms; myelogenous leukemia, chronic (cml); myeloid leukemia, acute (aml); myeloproliferative neoplasms, chronic; nasal cavity and paranasal sinus cancer (head and neck cancer); nasopharyngeal cancer (head and neck cancer); neuroblastoma; non-hodgkin lymphoma; non-small cell lung cancer; oral cancer, lip and oral cavity cancer and oropharyngeal cancer (head and neck cancer); osteosarcoma and malignant fibrous histiocytoma of bone; ovarian cancer; childhood ovarian cancer; pancreatic cancer; childhood pancreatic cancer; pancreatic neuroendocrine tumors (islet cell tumors); papillomatosis (childhood laryngeal); paraganglioma; childhood paraganglioma; paranasal sinus and nasal cavity cancer (head and neck cancer); parathyroid cancer; penile cancer; pharyngeal cancer (head and neck cancer); pheochromocytoma; childhood pheochromocytoma; pituitary tumor; plasma cell neoplasm/multiple myeloma; pleuropulmonary blastoma; pregnancy and breast cancer; primary central nervous system (CNS) lymphoma; primary peritoneal cancer; prostate cancer; rectal cancer; recurrent cancer; renal cell (kidney) cancer; retinoblastoma; rhabdomyosarcoma, childhood (soft tissue sarcoma); salivary gland cancer (head and neck cancer); sarcoma; childhood rhabdomyosarcoma (soft tissue sarcoma); childhood vascular tumors (soft tissue sarcoma); ewing sarcoma (bone cancer); kaposi sarcoma (soft tissue sarcoma); osteosarcoma (bone cancer); soft tissue sarcoma; uterine sarcoma; sézary syndrome (lymphoma); skin cancer; childhood skin cancer; small cell lung cancer; small intestine cancer; soft tissue sarcoma; squamous cell carcinoma of the skin—see skin cancer; squamous neck cancer with occult primary, metastatic (head and neck cancer); stomach (gastric) cancer; childhood stomach (gastric) cancer; t-cell lymphoma, cutaneous—see lymphoma (mycosis fungoides and sèzary syndrome); testicular cancer; childhood testicular cancer; throat cancer (head and neck cancer); nasopharyngeal cancer; oropharyngeal cancer; hypopharyngeal cancer; thymoma and thymic carcinoma; thyroid cancer; transitional cell cancer of the renal pelvis and ureter (kidney (renal cell) cancer); carcinoma of unknown primary; childhood cancer of unknown primary; unusual cancers of childhood; ureter and renal pelvis, transitional cell cancer (kidney (renal cell) cancer; urethral cancer; uterine cancer, endometrial; uterine sarcoma; vaginal cancer; childhood vaginal cancer; vascular tumors (soft tissue sarcoma); vulvar cancer; wilms tumor and other childhood kidney tumors; or cancer in young adults.

The methods, compositions, and systems of the present disclosure may be used to detect a cardiovascular disease state. As used herein, the terms “cardiovascular disease” (CVD) or “cardiovascular disorder” are used to classify numerous conditions affecting the heart, heart valves, and vasculature (e.g., veins and arteries) of the body and encompasses diseases and conditions including, but not limited to atherosclerosis, myocardial infarction, acute coronary syndrome, angina, congestive heart failure, aortic aneurysm, aortic dissection, iliac or femoral aneurysm, pulmonary embolism, atrial fibrillation, stroke, transient ischemic attack, systolic dysfunction, diastolic dysfunction, myocarditis, atrial tachycardia, ventricular fibrillation, endocarditis, peripheral vascular disease, and coronary artery disease (CAD). Further, the term cardiovascular disease refers to conditions in subjects that ultimately have a cardiovascular event or cardiovascular complication, referring to the manifestation of an adverse condition in a subject brought on by cardiovascular disease, such as sudden cardiac death or acute coronary syndrome, including, but not limited to, myocardial infarction, unstable angina, aneurysm, stroke, heart failure, non-fatal myocardial infarction, stroke, angina pectoris, transient ischemic attacks, aortic aneurysm, aortic dissection, cardiomyopathy, abnormal cardiac catheterization, abnormal cardiac imaging, stent or graft revascularization, risk of experiencing an abnormal stress test, risk of experiencing abnormal myocardial perfusion, and death.

As used herein, the ability to detect, diagnose or prognose cardiovascular disease, for example, atherosclerosis, can include determining if the patient is in a pre-stage of cardiovascular disease, has developed early, moderate or severe forms of cardiovascular disease, or has suffered one or more cardiovascular event or complication associated with cardiovascular disease.

Atherosclerosis (also known as arteriosclerotic vascular disease or ASVD) is a cardiovascular disease in which an artery-wall thickens as a result of invasion and accumulation and deposition of arterial plaques containing white blood cells on the innermost layer of the walls of arteries resulting in the narrowing and hardening of the arteries. The arterial plaque is an accumulation of macrophage cells or debris, and contains lipids (cholesterol and fatty acids), calcium and a variable amount of fibrous connective tissue. Diseases associated with atherosclerosis include, but are not limited to, atherothrombosis, coronary heart disease, deep venous thrombosis, carotid artery disease, angina pectoris, peripheral arterial disease, chronic kidney disease, acute coronary syndrome, vascular stenosis, myocardial infarction, aneurysm or stroke. In one embodiment the automated apparatuses, compositions, and methods of the present disclosure may distinguish the different stages of atherosclerosis, including, but not limited to, the different degrees of stenosis in a subject.

In some embodiments, the disease or disorder detected by the methods, compositions, or systems of the present disclosure is an endocrine disease. The term “endocrine disease” is used to refer to a disorder associated with dysregulation of endocrine system of a subject. Endocrine diseases may result from a gland producing too much or too little of an endocrine hormone causing a hormonal imbalance, or due to the development of lesions (such as nodules or tumors) in the endocrine system, which may or may not affect hormone levels. Suitable endocrine diseases able to be treated include, but are not limited to, e.g., Acromegaly, Addison's Disease, Adrenal Cancer, Adrenal Disorders, Anaplastic Thyroid Cancer, Cushing's Syndrome, De Quervain's Thyroiditis, Diabetes, Follicular Thyroid Cancer, Gestational Diabetes, Goiters, Graves' Disease, Growth Disorders, Growth Hormone Deficiency, Hashimoto's Thyroiditis, Hurthle Cell Thyroid Cancer, Hyperglycemia, Hyperparathyroidism, Hyperthyroidism, Hypoglycemia, Hypoparathyroidism, Hypothyroidism, Low Testosterone, Medullary Thyroid Cancer, MEN 1, MEN 2A, MEN 2B, Menopause, Metabolic Syndrome, Obesity, Osteoporosis, Papillary Thyroid Cancer, Parathyroid Diseases, Pheochromocytoma, Pituitary Disorders, Pituitary Tumors, Polycystic Ovary Syndrome, Prediabetes, Silent, Thyroiditis, Thyroid Cancer, Thyroid Diseases, Thyroid Nodules, Thyroiditis, Turner Syndrome, Type 1 Diabetes, Type 2 Diabetes, and the like.

In some embodiments, the disease or disorder detected by methods, compositions, or systems of the present disclosure is an inflammatory disease. As referred to herein, inflammatory disease refers to a disease caused by uncontrolled inflammation in the body of a subject. Inflammation is a biological response of the subject to a harmful stimulus which may be external or internal such as pathogens, necrosed cells and tissues, irritants etc. However, when the inflammatory response becomes abnormal, it results in self-tissue injury and may lead to various diseases and disorders. Inflammatory diseases can include, but are not limited to, asthma, glomerulonephritis, inflammatory bowel disease, rheumatoid arthritis, hypersensitivities, pelvic inflammatory disease, autoimmune diseases, arthritis; necrotizing enterocolitis (NEC), gastroenteritis, pelvic inflammatory disease (PID), emphysema, pleurisy, pyelitis, pharyngitis, angina, acne vulgaris, urinary tract infection, appendicitis, bursitis, colitis, cystitis, dermatitis, phlebitis, rhinitis, tendonitis, tonsillitis, vasculitis, autoimmune diseases; celiac disease; chronic prostatitis, hypersensitivities, reperfusion injury; sarcoidosis, transplant rejection, vasculitis, interstitial cystitis, hay fever, periodontitis, atherosclerosis, psoriasis, ankylosing spondylitis, juvenile idiopathic arthritis, Behcet's disease, spondyloarthritis, uveitis, systemic lupus erythematosus, and cancer. For example, the arthritis includes rheumatoid arthritis, psoriatic arthritis, osteoarthritis or juvenile idiopathic arthritis, and the like.

The methods, compositions, and systems of the present disclosure may detect a neurological disease state. Neurological disorders or neurological diseases are used interchangeably and refer to diseases of the brain, spine and the nerves that connect them. Neurological diseases include, but are not limited to, brain tumors, epilepsy, Parkinson's disease, Alzheimer's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease (including but are not limited to, multiple sclerosis, Devic's disease (i.e. neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy) and the like. Neurological disorders also include immune-mediated neurological disorders (IMNDs), which include diseases with at least one component of the immune system reacts against host proteins present in the central or peripheral nervous system and contributes to disease pathology. IMNDs may include, but are not limited to, demyelinating disease, paraneoplastic neurological syndromes, immune-mediated encephalomyelitis, immune-mediated autonomic neuropathy, myasthenia gravis, autoantibody-associated encephalopathy, and acute disseminated encephalomyelitis.

Methods, systems, and/or apparatuses of the present disclosure may be able to accurately distinguish between patients with or without Alzheimer's disease. These may also be able to detect patients who are pre-symptomatic and may develop Alzheimer's disease several years after the screening. This provides advantages of being able to treat a disease at a very early stage, even before development of the disease.

The methods, compositions, and systems of the present disclosure can detect a pre-disease stage of a disease or disorder. A pre-disease stage is a stage at which the patient has not developed any signs or symptoms of the disease. A pre-cancerous stage would be a stage in which cancer or tumor or cancerous cells have not be identified within the subject. A pre-neurological disease stage would be a stage in which a person has not developed one or more symptom of the neurological disease. The ability to diagnose a disease before one or more sign or symptom of the disease is present allows for close monitoring of the subject and the ability to treat the disease at a very early stage, increasing the prospect of being able to halt progression or reduce the severity of the disease.

The methods, compositions, and systems of the present disclosure may detect the early stages of a disease or disorder. Early stages of the disease can refer to when the first signs or symptoms of a disease may manifest within a subject. The early stage of a disease may be a stage at which there are no outward signs or symptoms. For example, in Alzheimer's disease an early stage may be a pre-Alzheimer's stage in which no symptoms are detected yet the patient will develop Alzheimer's months or years later.

Identifying a disease in either pre-disease development or in the early states may often lead to a higher likelihood for a positive outcome for the patient. For example, diagnosing cancer at an early stage (stage 0 or stage 1) can increase the likelihood of survival by over 80%. Stage 0 cancer can describe a cancer before it has begun to spread to nearby tissues. This stage of cancer is often highly curable, usually by removing the entire tumor with surgery. Stage 1 cancer may usually be a small cancer or tumor that has not grown deeply into nearby tissue and has not spread to lymph nodes or other parts of the body.

In some embodiments, the methods, compositions, and systems of the present disclosure are able to detect intermediate stages of the disease. Intermediate states of the disease describe stages of the disease that have passed the first signs and symptoms and the patient is experiencing one or more symptom of the disease. For example, for cancer, stage II or III cancers are considered intermediate stages, indicating larger cancers or tumors that have grown more deeply into nearby tissue. In some instances, stage II or III cancers may have also spread to lymph nodes but not to other parts of the body.

Further, the methods, compositions, and systems of the present disclosure may be able to detect late or advanced stages of the disease. Late or advanced stages of the disease may also be called “severe” or “advanced” and usually indicates that the subject is suffering from multiple symptoms and effects of the disease. For example, severe stage cancer includes stage IV, where the cancer has spread to other organs or parts of the body and is sometimes referred to as advanced or metastatic cancer.

The methods of the present disclosure can include processing the biomolecule corona data of a sample against a collection of biomolecule corona datasets representative of a plurality of diseases and/or a plurality of disease states to determine if the sample indicates a disease and/or disease state. For example, samples can be collected from a population of subjects over time. Once the subjects develop a disease or disorder, the present disclosure allows for the ability to characterize and detect the changes in biomolecule fingerprints over time in the subject by computationally analyzing the biomolecule fingerprint of the sample from the same subject before they have developed a disease to the biomolecule fingerprint of the subject after they have developed the disease. Samples can also be taken from cohorts of patients who all develop the same disease, allowing for analysis and characterization of the biomolecule fingerprints that are associated with the different stages of the disease for these patients (e.g. from pre-disease to disease states).

In some embodiments, the methods, compositions, and systems of the present disclosure are able to distinguish not only between different types of diseases, but also between the different stages of the disease (e.g., early stages of cancer). This can comprise distinguishing healthy subjects from pre-disease state subjects. The pre-disease state may be stage 0 or stage 1 cancer, a neurodegenerative disease, dementia, a coronary disease, a kidney disease, a cardiovascular disease (e.g., coronary artery disease), diabetes, or a liver disease. Distinguishing between different stages of the disease can comprise distinguishing between two stages of a cancer (e.g., stage 0 vs stage 1 or stage 1 vs stage 3).

### Computer Control Systems

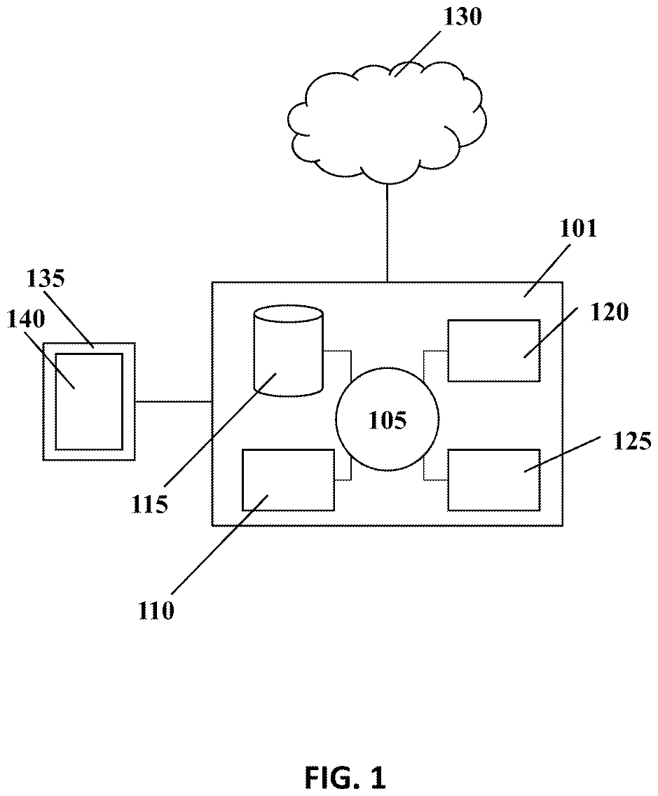

The present disclosure provides computer control systems that are programmed to implement methods of the disclosure. FIG. 1 shows a computer system that is programmed or otherwise configured to implement methods provided herein. The computer system 101 can regulate various aspects of the assays disclosed herein, which are capable of being automated (e.g., movement of any of the reagents disclosed herein on a substrate). The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some embodiments, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some embodiments, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example a readout of the proteins identified using the methods disclosed herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105.

Determination, analysis or statistical classification is done by methods known in the art, including, but not limited to, for example, a wide variety of supervised and unsupervised data analysis and clustering approaches such as hierarchical cluster analysis (HCA), principal component analysis (PCA), Partial least squares Discriminant Analysis (PLSDA), machine learning (also known as random forest), logistic regression, decision trees, support vector machine (SVM), k-nearest neighbors, naive bayes, linear regression, polynomial regression, SVM for regression, K-means clustering, and hidden Markov models, among others. The computer system can perform various aspects of analyzing the protein sets or protein corona of the present disclosure, such as, for example, comparing/analyzing the biomolecule corona of several samples to determine with statistical significance what patterns are common between the individual biomolecule coronas to determine a protein set that is associated with the biological state. The computer system can be used to develop classifiers to detect and discriminate different protein sets or protein corona (e.g., characteristic of the composition of a protein corona). Data collected from the presently disclosed sensor array can be used to train a machine learning algorithm, specifically an algorithm that receives array measurements from a patient and outputs specific biomolecule corona compositions from each patient. Before training the algorithm, raw data from the array can be first denoised to reduce variability in individual variables.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory. A computer system may be adapted to implement a method described herein. The system includes a central computer server that is programmed to implement the methods described herein. The server includes a central processing unit (CPU, also “processor”) which can be a single core processor, a multi core processor, or plurality of processors for parallel processing. The server also includes memory (e.g., random access memory, read-only memory, flash memory); electronic storage unit (e.g. hard disk); communications interface (e.g., network adaptor) for communicating with one or more other systems; and peripheral devices which may include cache, other memory, data storage, and/or electronic display adaptors. The memory, storage unit, interface, and peripheral devices are in communication with the processor through a communications bus (solid lines), such as a motherboard. The storage unit can be a data storage unit for storing data. The server is operatively coupled to a computer network (“network”) with the aid of the communications interface. The network can be the Internet, an intranet and/or an extranet, an intranet and/or extranet that is in communication with the Internet, a telecommunication or data network. The network In some embodiments, with the aid of the server, can implement a peer-to-peer network, which may enable devices coupled to the server to behave as a client or a server.

The storage unit can store files, such as subject reports, and/or communications with the data about individuals, or any aspect of data associated with the present disclosure.

The computer server can communicate with one or more remote computer systems through the network. The one or more remote computer systems may be, for example, personal computers, laptops, tablets, telephones, Smart phones, or personal digital assistants.

In some applications the computer system includes a single server. In other situations, the system includes multiple servers in communication with one another through an intranet, extranet and/or the internet.

The server can be adapted to store measurement data or a database as provided herein, patient information from the subject, such as, for example, medical history, family history, demographic data and/or other clinical or personal information of potential relevance to a particular application. Such information can be stored on the storage unit or the server and such data can be transmitted through a network.

Methods as described herein can be implemented by way of machine (or computer processor) executable code (or software) stored on an electronic storage location of the server, such as, for example, on the memory, or electronic storage unit. During use, the code can be executed by the processor. In some embodiments, the code can be retrieved from the storage unit and stored on the memory for ready access by the processor. In some situations, the electronic storage unit can be precluded, and machine-executable instructions are stored on memory. Alternatively, the code can be executed on a second computer system.

Aspects of the systems and methods provided herein, such as the server, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide nontransitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Classification of Protein Corona Using Machine Learning

The method of determining a set of proteins associated with the disease or disorder and/or disease state include the analysis of the corona of the at least two samples. This determination, analysis or statistical classification is done by methods known in the art, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive bayes, linear regression, polynomial regression, SVM for regression, K-means clustering, and hidden Markov models, among others. In other words, the proteins in the corona of each sample are compared/analyzed with each other to determine with statistical significance what patterns are common between the individual corona to determine a set of proteins that is associated with the disease or disorder or disease state.

Generally, machine learning algorithms are used to construct models that accurately assign class labels to examples based on the input features that describe the example. In some case it may be advantageous to employ machine learning and/or deep learning approaches for the methods described herein. For example, machine learning can be used to associate the protein corona with various disease states (e.g. no disease, precursor to a disease, having early or late stage of the disease, etc.). For example, In some embodiments, one or more machine learning algorithms are employed in connection with a method of the invention to analyze data detected and obtained by the protein corona and sets of proteins derived therefrom. For example, in one embodiment, machine learning can be coupled with the sensor array described herein to determine not only if a subject has a pre-stage of cancer, cancer or does not have or develop cancer, but also to distinguish the type of cancer.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### Example 1: Synthesis of Peptide Functionalized Surfaces

SMCC Route for Peptide Functionalization: A fresh sulfo-SMCC stock solution is prepared at 1 mg/mL in diH2O. Sonication and inversion helps to fully dissolve the powder. The volume may vary based on mass of SMCC used. 2-mL and/or 5 mL centrifuge tubes can be used. 100 mg of feed nanoparticles with 19.6 mL of 1×PBS (pH 7.4, 2 mM) in a 40-mL glass vial are resuspended with brief sonication. 400 μL of sulfo-SMCC stock solution is added to feed and vortex-mixed. The vial is rotated at 50 rpm for 2 h. The contents are washed with PBS twice and resuspended in 19 mL PBS (brief sonication may be needed).

A fresh peptide solution at 5 mg/mL is prepared in PBS. For one synthesis, the volume may vary based on how mass of peptide used. 2-mL and/or 5 mL centrifuge tubes can be used. 1 mL of the peptide solution is added to the diluted feed and gently mix with vortex. The tube is rotated at 50 rpm for 4 h. The contents are washed with PBS twice. NPs are resuspended in 0.01 mg/mL peptide solution for storage (aggregations should break down with extra peptide).

Click Route of Peptide Functionalization: Azide functionalized nanoparticles are magnetically washed 3 times in DMF to remove any potential loose molecules and then they are mixed with DMF or ethanol-based solution of 20× (mass ratio) DBCO functionalized molecules of choice (e.g., DBCO-amine) on a shaker to homogenize. The mix is kept at 50° C. for 4-24 hours on medium rate shaking to push for the maximum reaction efficiency. The final particles are quenched by DMF washing several times after the reaction completion and resuspended in ethanol for further steps.

The synthesized particles are shown in Tables 2-4.

TABLE 2
Characterization data of particles synthesized
from feed lot S-439 compared to the feed lot.

| | Dynamic Light | | | | | | | |
|---|---|---|---|---|---|---|---|---|
| | Scattering | | | | | | | |
| | Measurements | Zeta Potential | X-Ray Photoelectron | | | | | |
| | Polydispersity | Measurements | Spectroscopy Measurements | | | | | |
| ID | Index | millivolts | % C | % O | % N | % S | % Si | % Fe |
| S-348-002 | 0.318 | 14.1 | 42.3 | 38.4 | 2.8 | 1.6 | 10.7 | 4.2 |
| S-348-037 | 0.438 | 16.9 | 20.8 | 48.6 | 3.6 | 1.4 | 24.7 | 0.0 |
| S-348-040 | 0.325 | 20.7 | 16.6 | 52.6 | 1.9 | 0.6 | 27.4 | 0.0 |
| S-348-041 | 0.343 | 20.3 | 17.5 | 51.8 | 1.9 | 0.6 | 27.2 | 0.0 |
| S-348-050 | 0.385 | 22.3 | 18.9 | 50.5 | 2.6 | 0.8 | 26.2 | 0.0 |
| S-348-051 | 0.445 | 20.6 | 19.4 | 50.1 | 2.8 | 0.9 | 26.0 | 0.0 |
| S-348-052 | 0.395 | 21.0 | 18.1 | 51.2 | 2.5 | 0.7 | 26.5 | 0.0 |
| S-348-053 | 0.327 | 22.3 | 18.6 | 50.7 | 2.5 | 0.8 | 26.4 | 0.0 |
| Mean | | | 18.6 | 50.8 | 2.5 | 0.8 | 26.4 | |
| S-439-004 | | | 15.0 | 53.8 | 1.3 | 0.4 | 28.4 | |
| (Feed) | | | | | | | | |
| S-439-005 | | | 14.9 | 53.9 | 1.3 | 0.6 | 28.3 | |
| (Feed) | | | | | | | | |
| Mean | | | 15.0 | 53.9 | 1.3 | 0.5 | 28.3 | |

TABLE 3
Characterization data of particles synthesized
from feed lot S-408 compared to the feed lot.

| | Dynamic Light | | | | | | | |
|---|---|---|---|---|---|---|---|---|
| | Scattering | | | | | | | |
| | Measurements | Zeta Potential | X-Ray Photoelectron | | | | | |
| | Polydispersity | Measurements | Spectroscopy Measurements | | | | | |
| ID | Index | millivolts | % C | % O | % N | % S | % Si | % Fe |
| S-348-046 | 0.290 | 12.0 | 22.2 | 48.5 | 3.6 | 1.3 | 24.5 | 0.0 |
| S-348-047 | 0.438 | 16.2 | 21.6 | 48.8 | 3.5 | 1.3 | 24.9 | 0.0 |
| S-348-048 | 0.333 | 13.6 | 22.0 | 48.4 | 3.9 | 1.3 | 24.3 | 0.0 |
| S-348-049 | 0.336 | 13.4 | 22.4 | 48.3 | 3.8 | 1.2 | 24.4 | 0.0 |
| Mean | | | 22.0 | 48.5 | 3.7 | 1.3 | 24.5 | |
| S-308-069 | | | 11.8 | 56.7 | 0.0 | 1.4 | 30.1 | |
| (Feed) | | | | | | | | |
| S-308-070 | | | 10.0 | 57.8 | 0.0 | 1.8 | 30.4 | |
| (Feed) | | | | | | | | |
| S-308-071 | | | 10.3 | 57.9 | 0.0 | 1.5 | 30.3 | |
| (Feed) | | | | | | | | |
| S-308-072 | | | 10.3 | 57.8 | 0.0 | 1.5 | 30.3 | |
| (Feed) | | | | | | | | |
| Mean | | | 10.6 | 57.5 | 0.0 | 1.5 | 30.3 | |

TABLE 4
Characterization data of particles synthesized
from feed lot S-437 compared to the feed lot.

| | Dynamic Light | | | | | | | |
|---|---|---|---|---|---|---|---|---|
| | Scattering | | | | | | | |
| | Measurements | Zeta Potential | X-Ray Photoelectron | | | | | |
| | Polydispersity | Measurements | Spectroscopy Measurements | | | | | |
| ID | Index | millivolts | % C | % O | % N | % S | % Si | % Fe |
| S-348-054 | 0.252 | 30.6 | 16.0 | 53.5 | 1.5 | 0.5 | 27.8 | 0.0 |
| S-348-055 | 0.235 | 30.8 | 15.7 | 53.8 | 1.7 | 0.5 | 27.7 | 0.0 |
| Mean | | | 15.8 | 53.7 | 1.6 | 0.5 | 27.7 | |
| S-437-003 (Feed) | | | 11.6 | 56.4 | 1.2 | 0.7 | 29.4 | |
| S-437-004 (Feed) | | | 12.6 | 56.1 | 1.2 | 0.5 | 29.1 | |
| Mean | | | 12.1 | 56.2 | 1.2 | 0.6 | 29.3 | |

### Example 2: Biomolecule Assay with Peptide Functionalized Surfaces

This example provides a method for performing a biomolecule assay with peptide functionalized surfaces.

Materials analysis using XPS: XPS (X-ray photoelectron spectroscopy) is a surface-sensitive analytical technique can be used to identify and quantify the elements that exist within ˜10-15 nm at the surface of a material or substrate. An XPS spectrum gives information about the composition of elements at the surface as well as their chemical states. XPS analysis of the NP samples typically includes a sample prep and powder mounting step followed by three survey scans (3 min) at ˜150 eV to identify the composition of elements that are present at the surface. High-resolution scans will also provide additional information on the chemical states and electronic structure of individual elements at the surface.

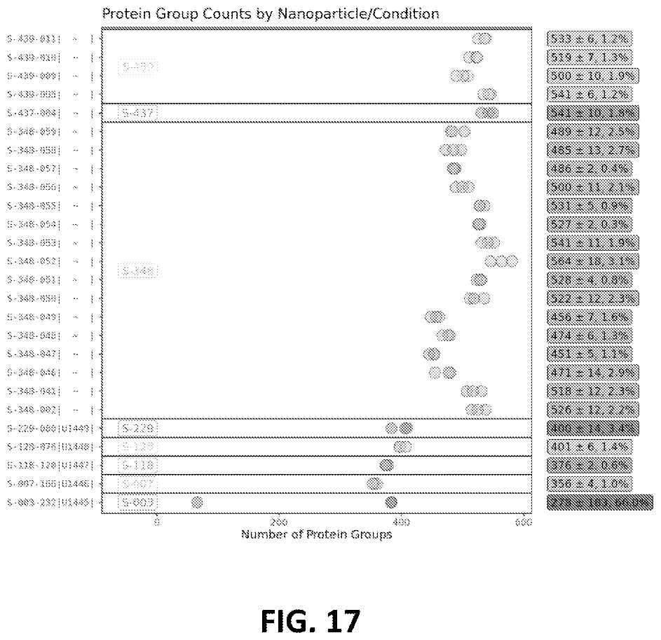

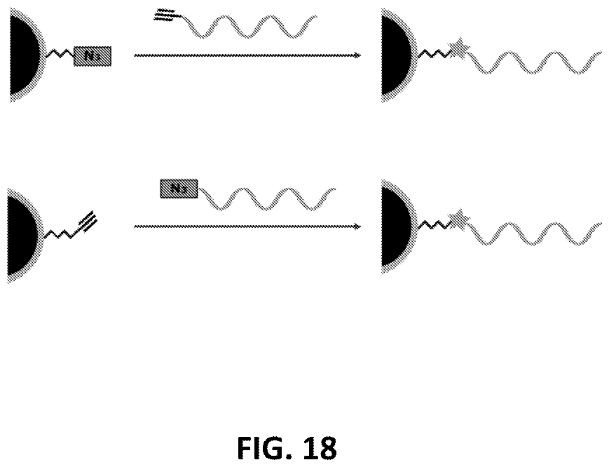

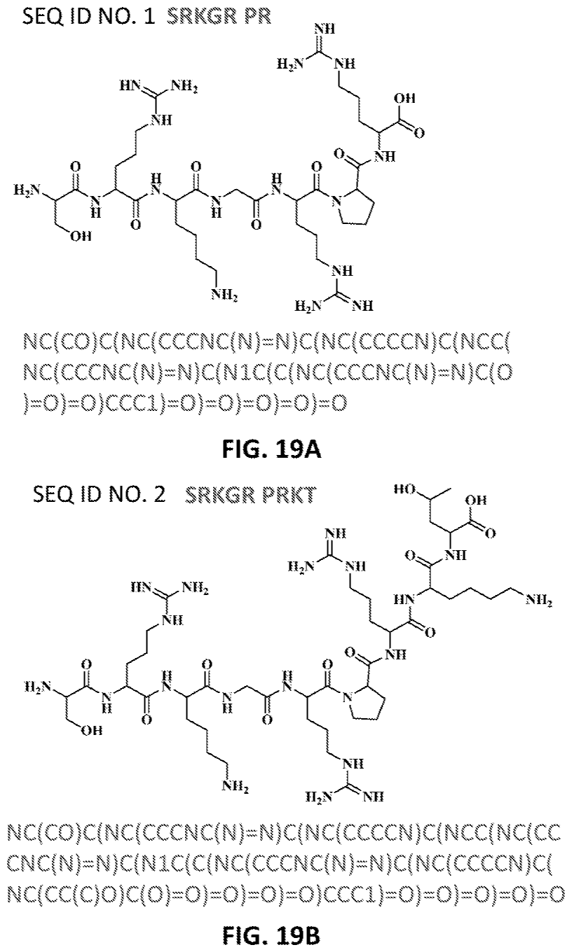

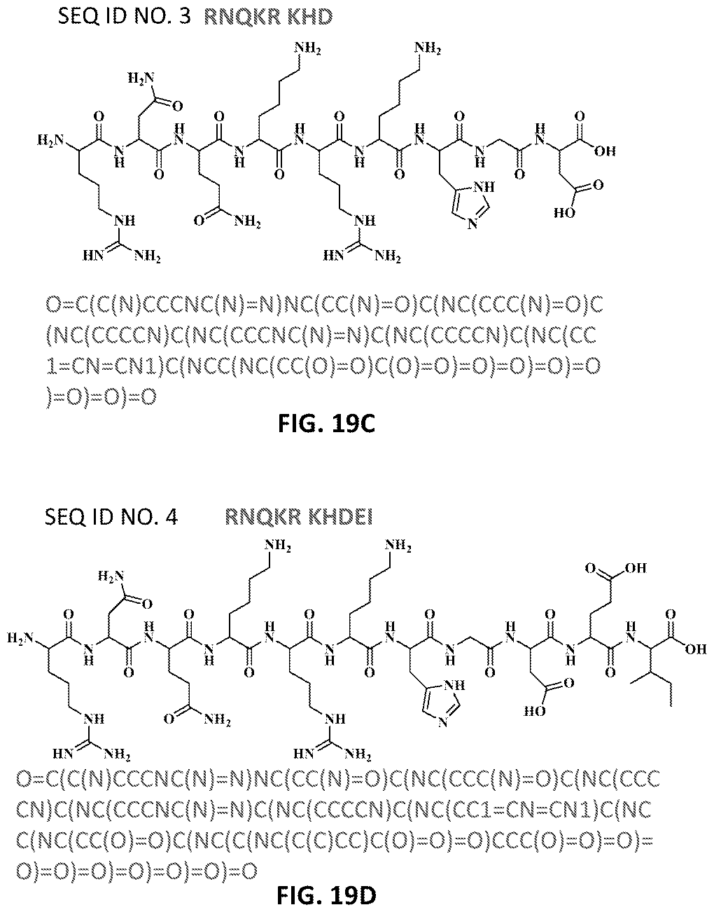

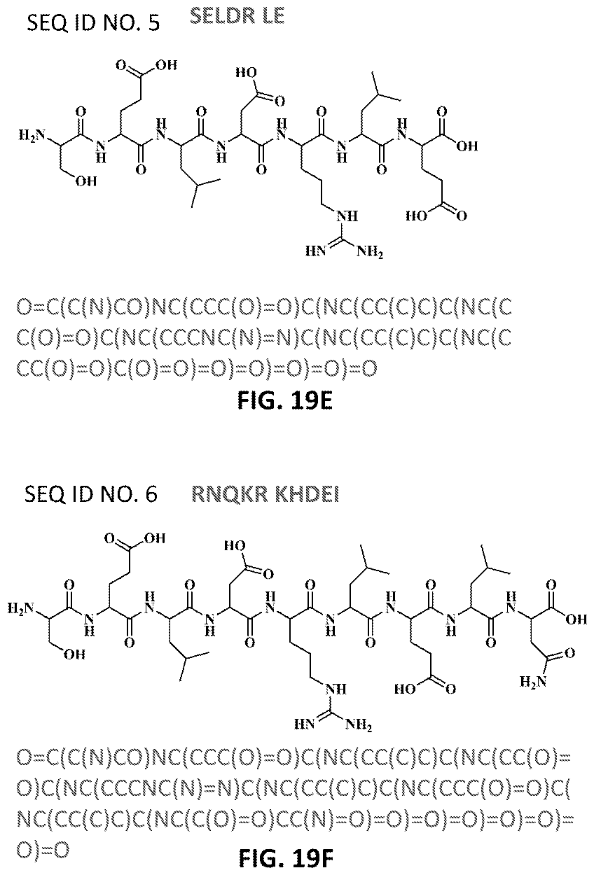

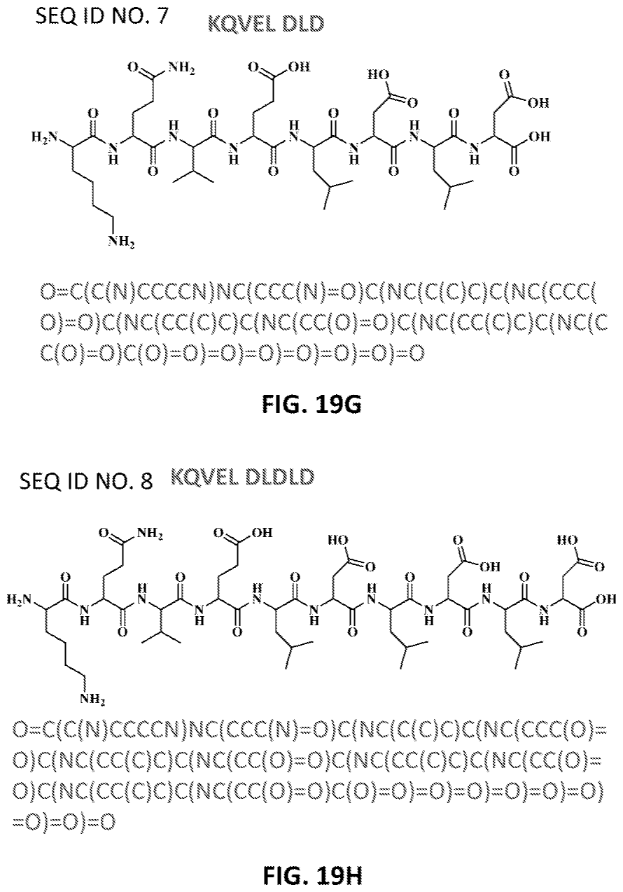

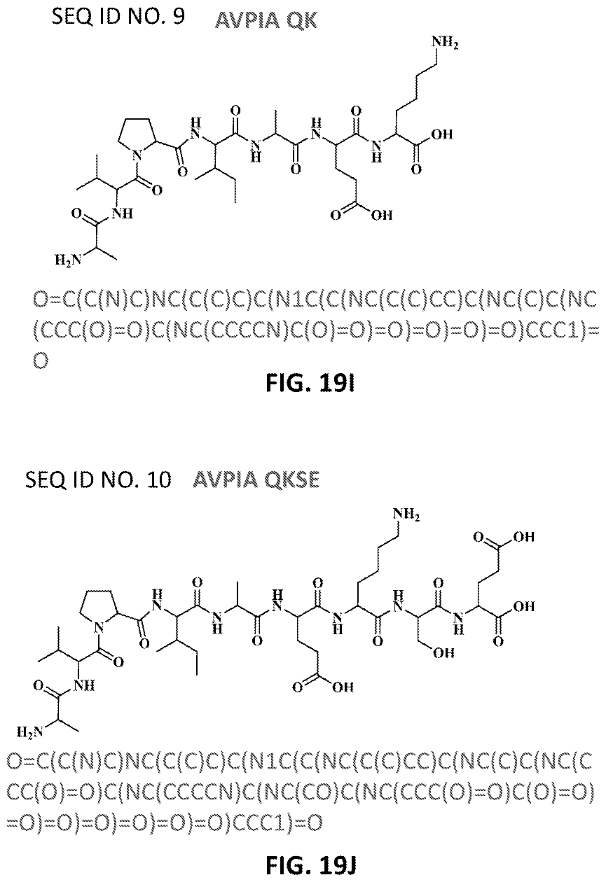

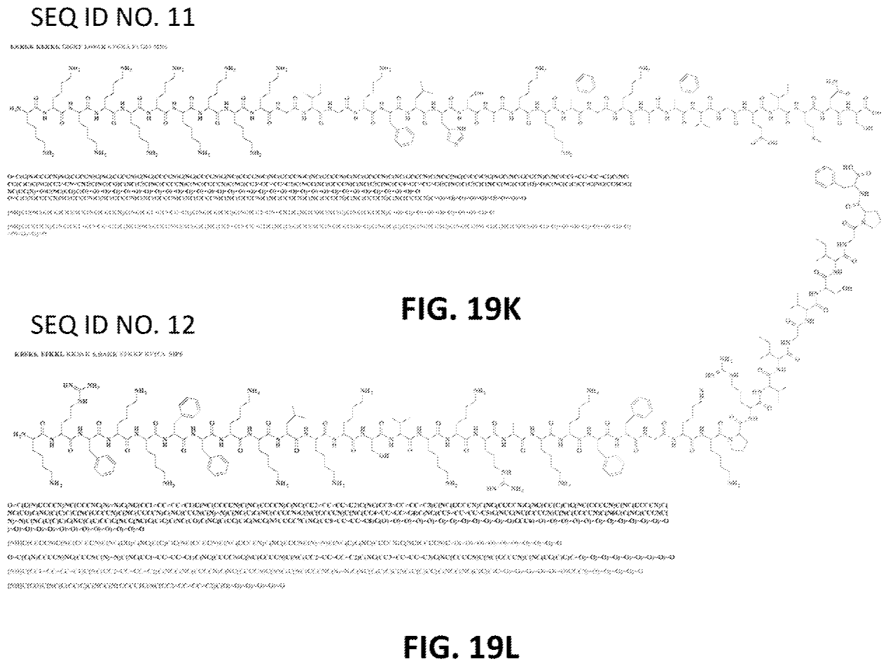

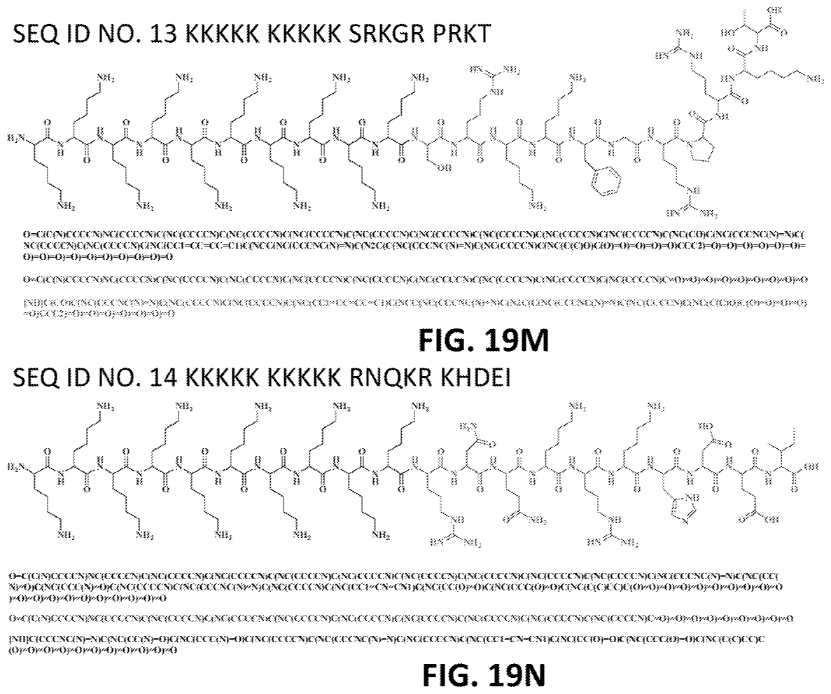

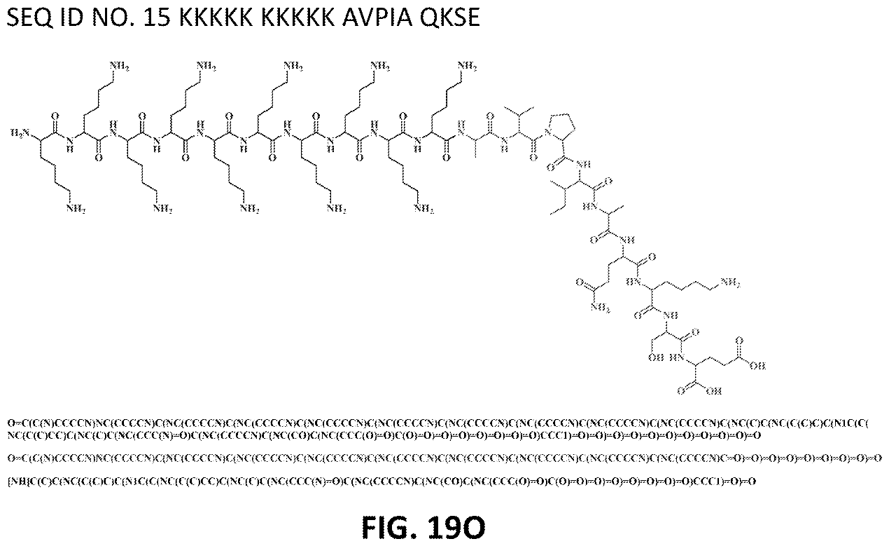

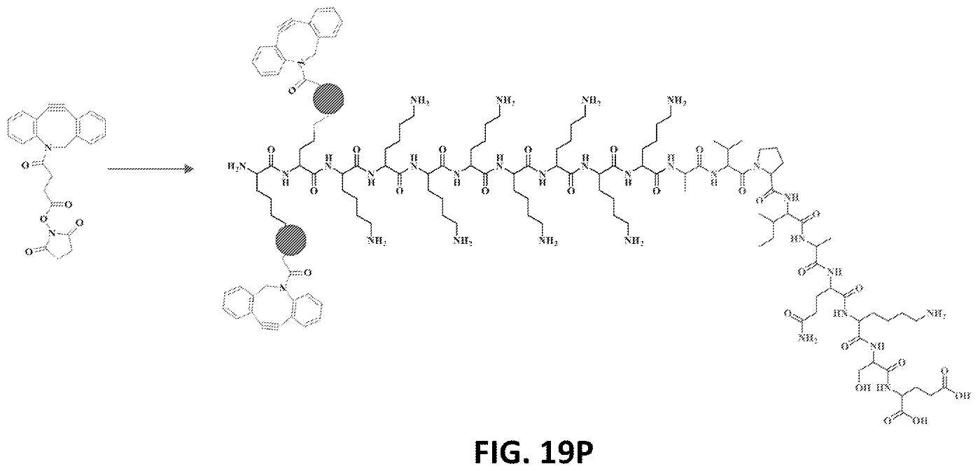

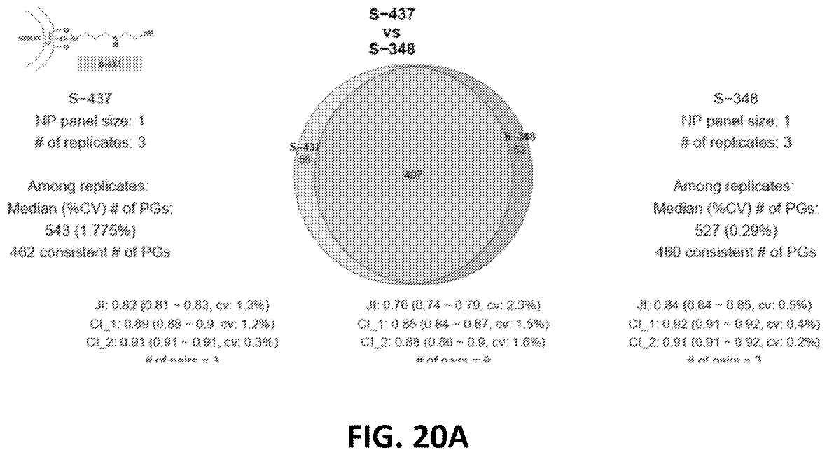

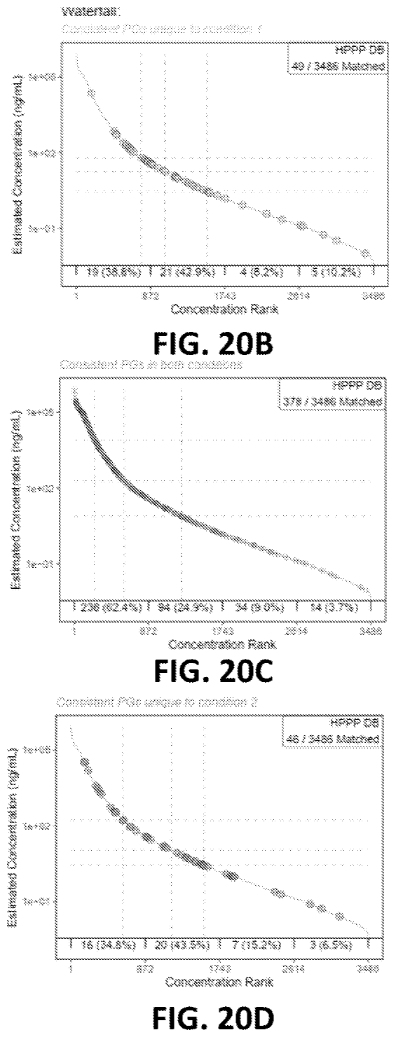

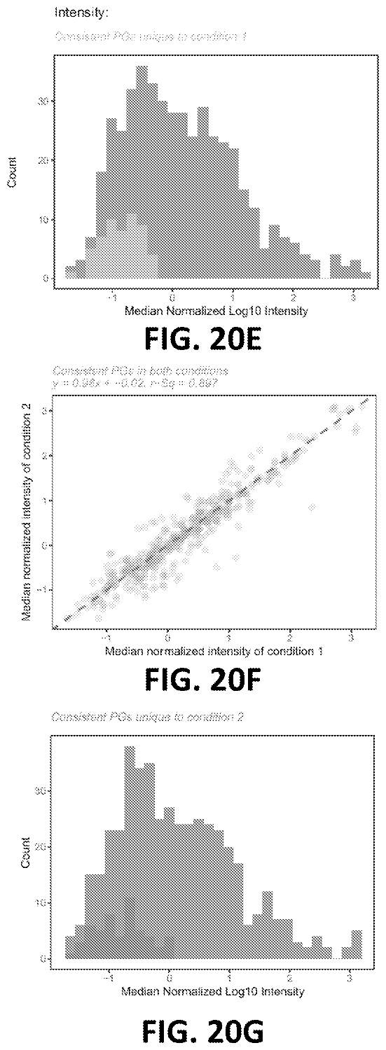

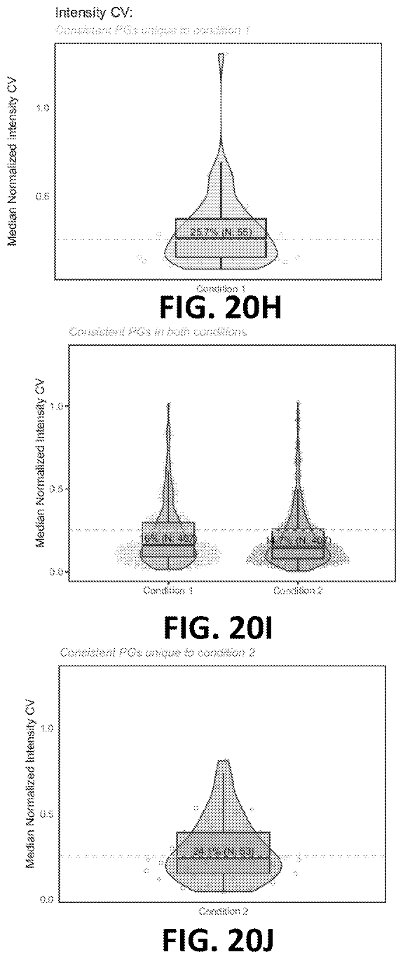

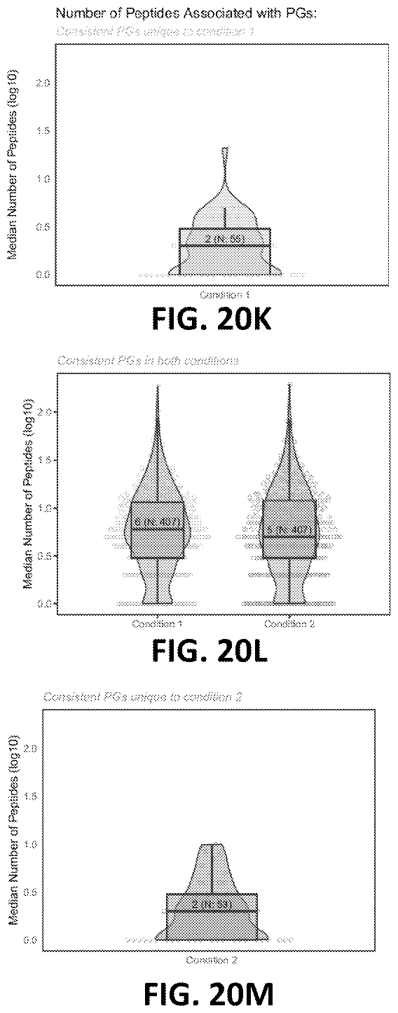

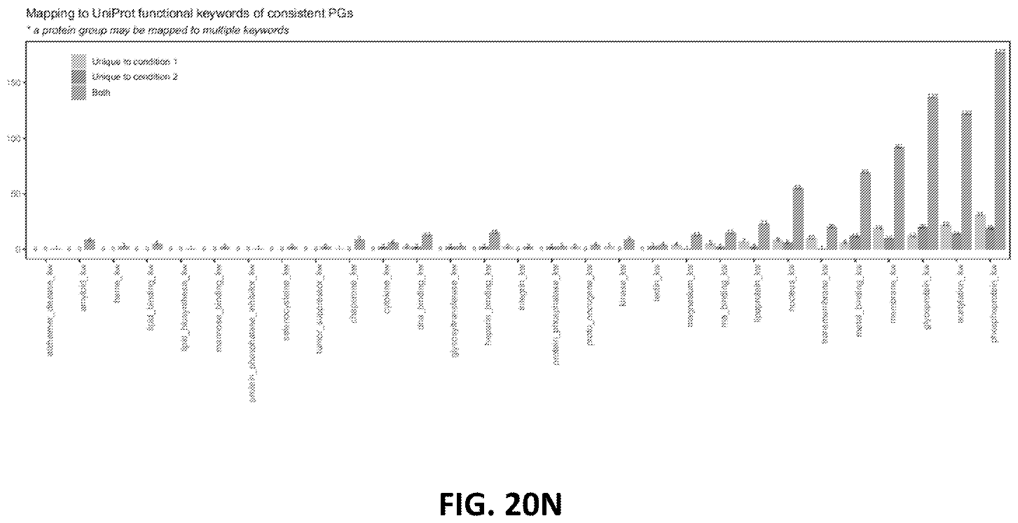

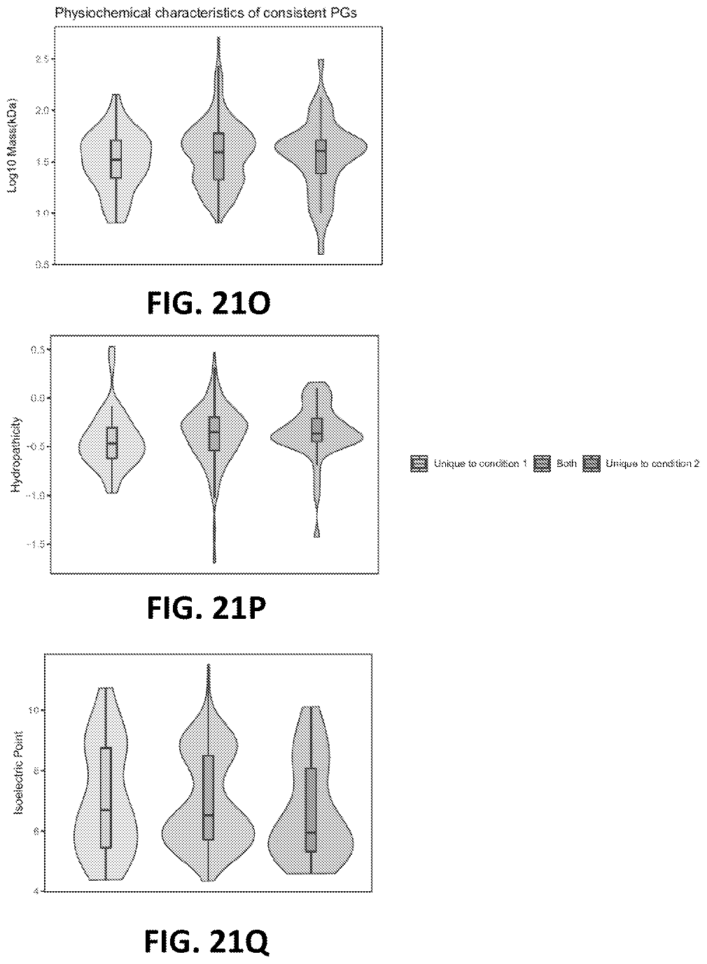

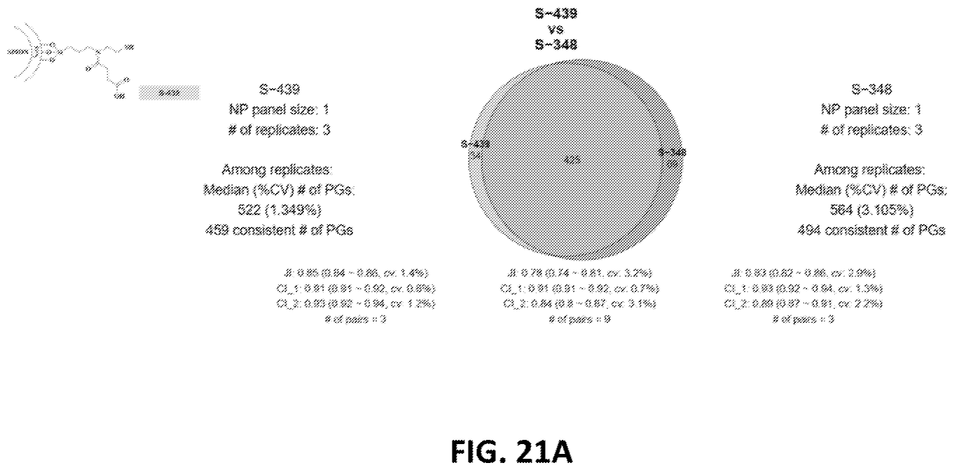

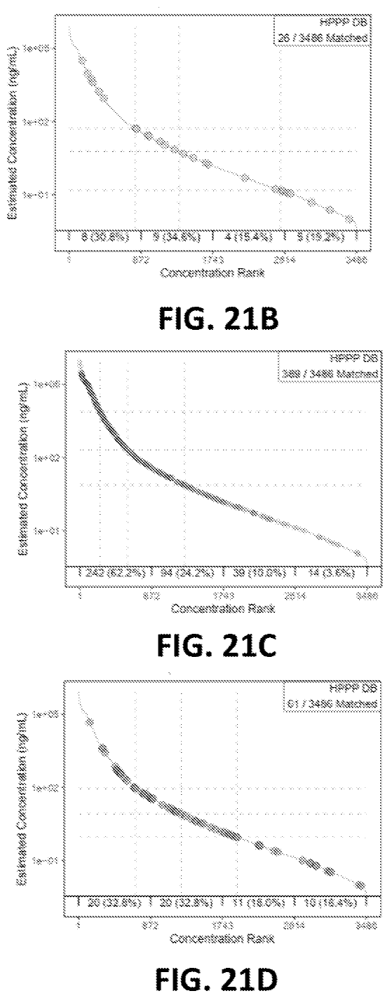

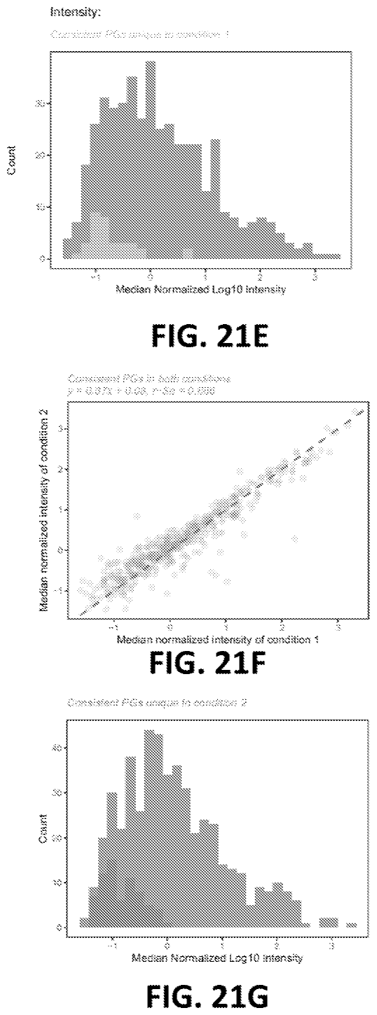

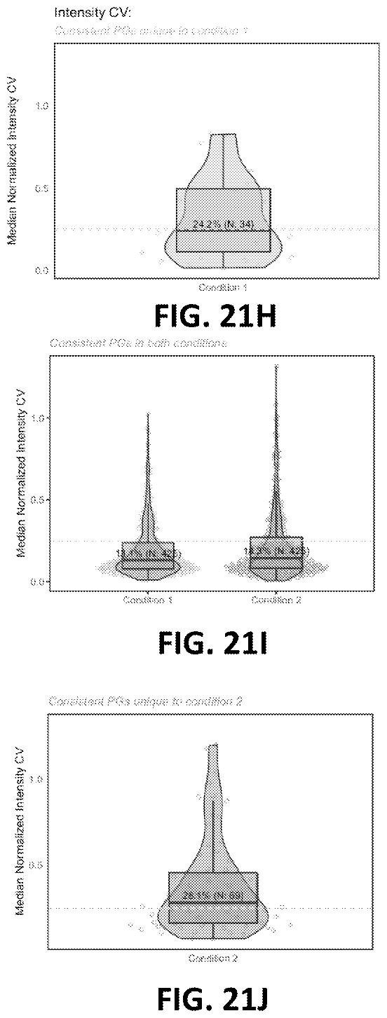

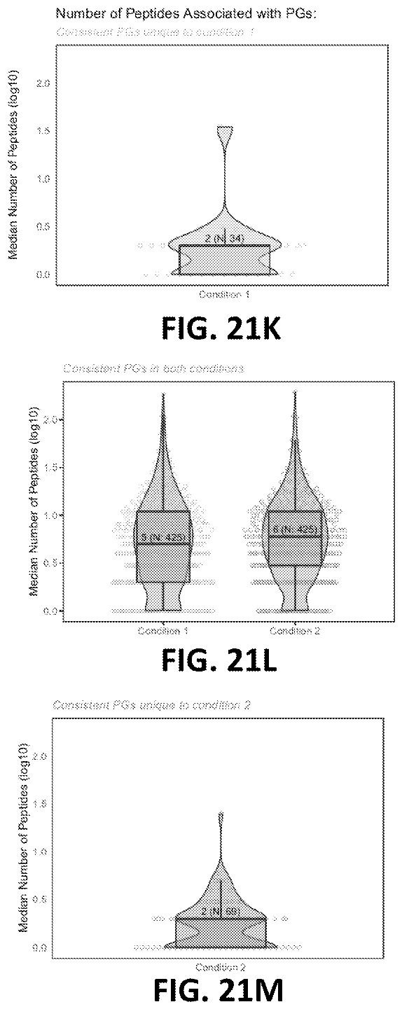

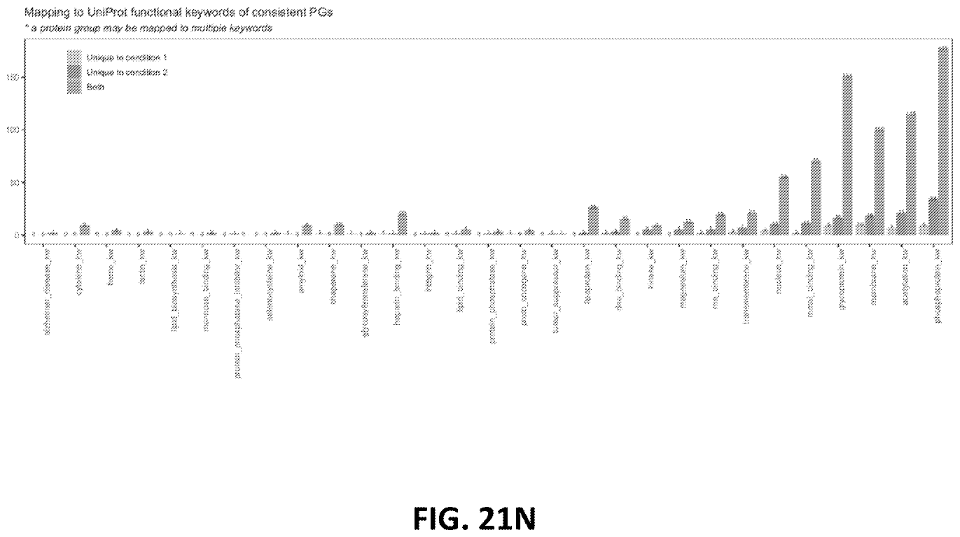

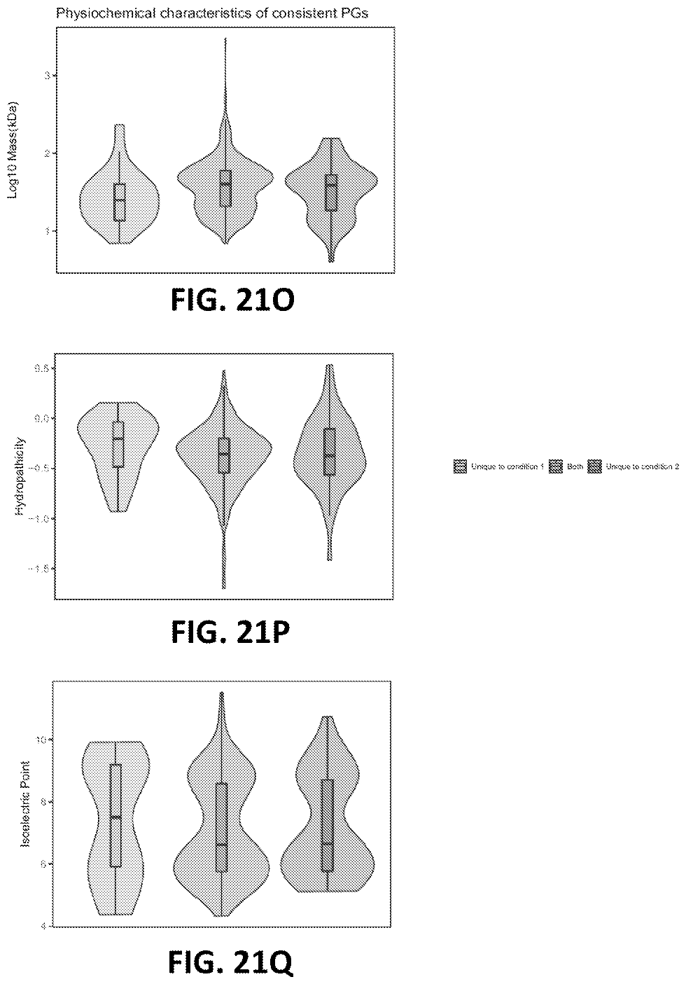

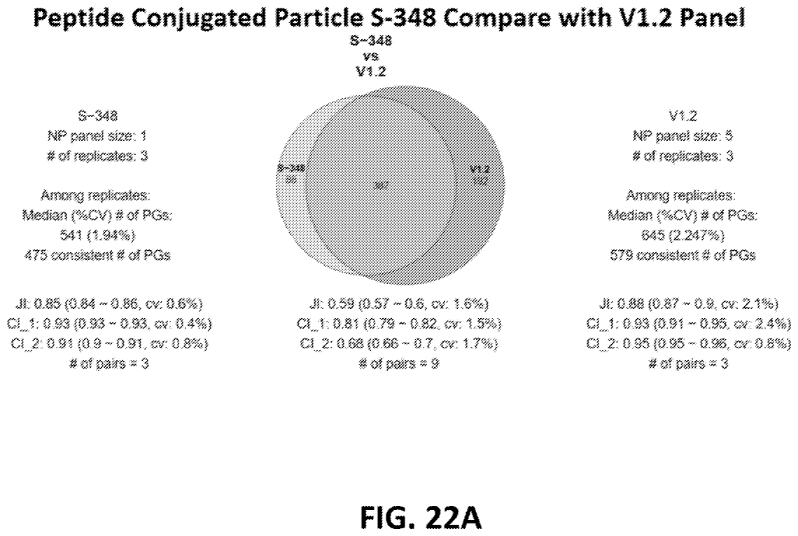

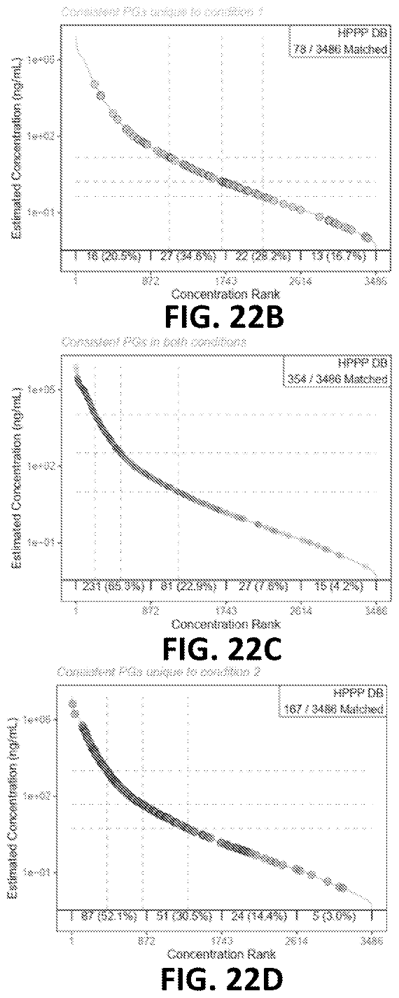

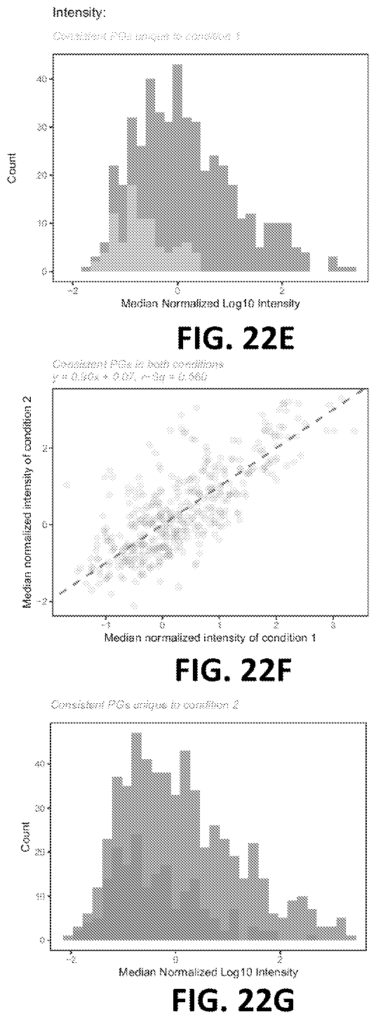

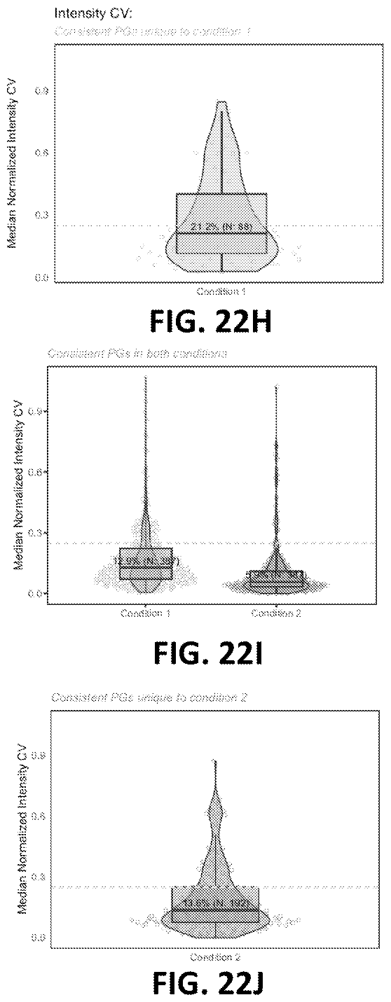

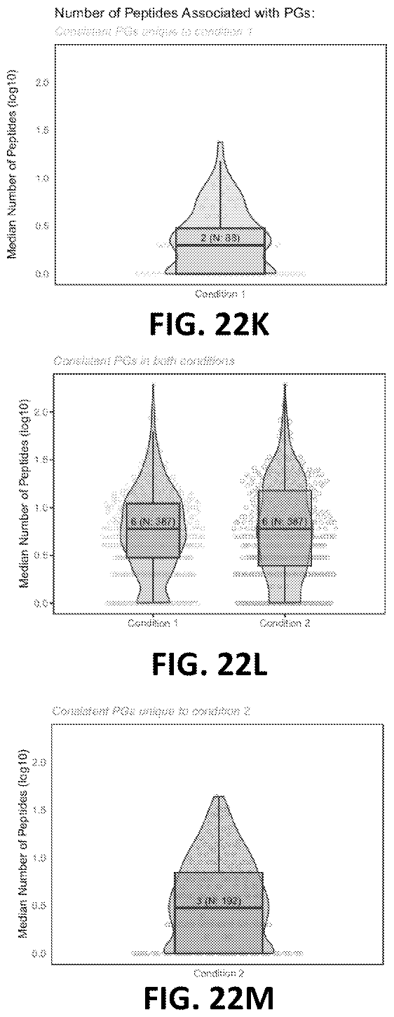

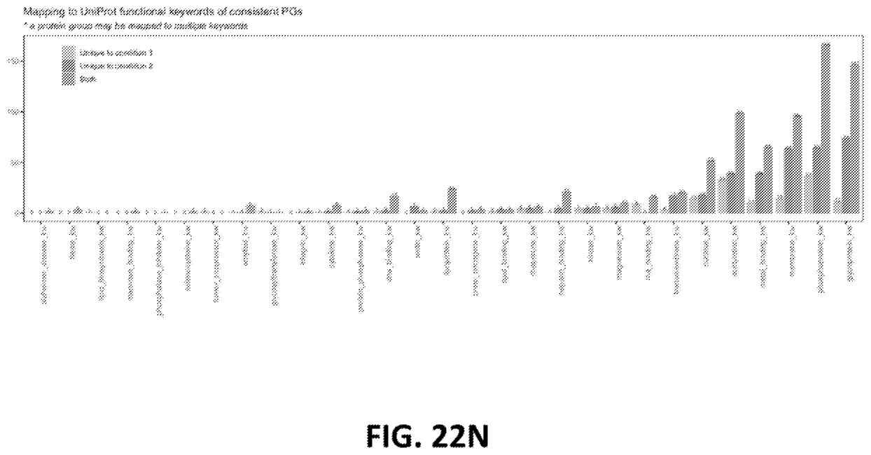

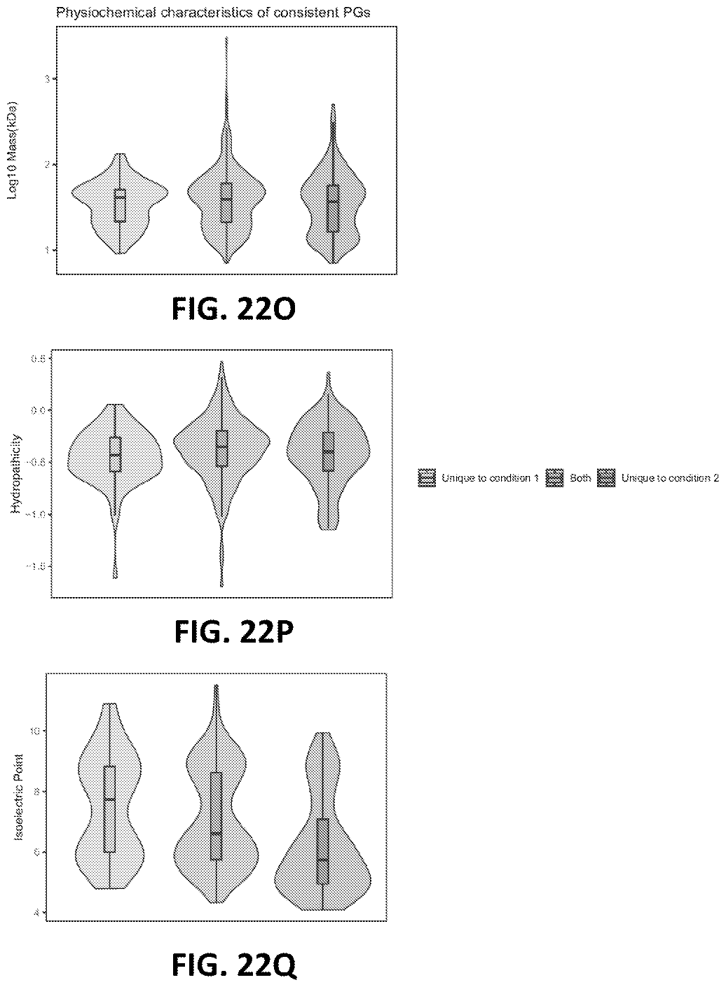

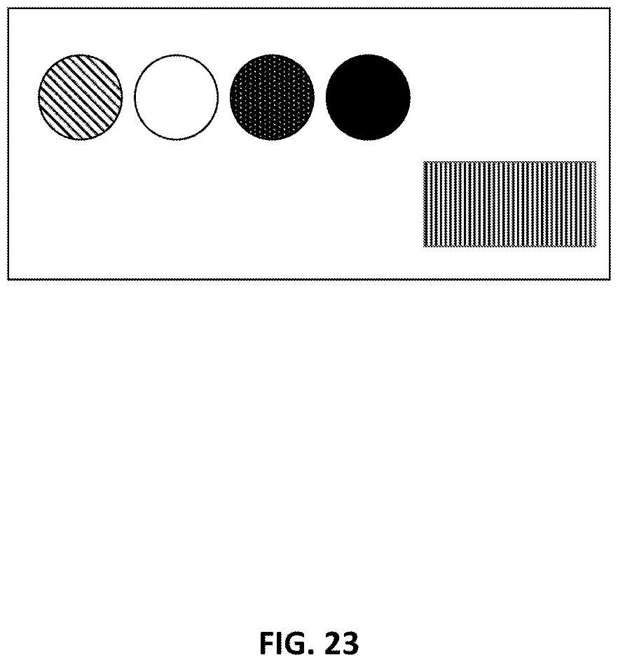

Proteograph and data analysis: The peptide conjugated NPs are used as designed in the Proteograph workflow without method modification. Briefly, NPs are incubated with biosample (plasma) for 30-60 minutes, washed to remove unbound proteins, and then subjected to tryptic digestion. Tryptic peptides are isolated and prepared for LC-MS/MS. The data is then processed to evaluate the specific proteins, with FIG. 17 representing the total protein groups detected for each NP. The number of protein groups identified ranged between 400 and 600. Each particle was able to capture a distinct biomolecule from another, as shown in FIGS. 20-22.

### Example 3: Prophetic Example for Peptide Design

This example provides a prophetic example for designing a peptide sequence.

One can begin by selected a protein target of interest. The protein target of interest may be, for example, a protein group associated with or suspected of being associated with a cancer.

In various individuals, proteins in the protein group may be expressed differently. For instance, between individuals, the proteins may have mutations, splicing variations, post-translational modifications, misfolding, or any other change.

One can search a protein database to find a protein or peptide that is known to bind to one protein in the protein group. The appropriate database may be Protein Data Bank.

Variations in the sequence of the protein or peptide may be considered for screening. Variations may include: truncating, extending, mutating, or chemically modifying the protein or peptide sequence. The protein or peptide may be altered at or in proximity to the binding site for binding. The protein or peptide may be at a location that is far from the binding site for binding. The variations may be screened using an in silico screening to select those that have the highest expected performance for binding multiple proteins in the protein group.

The in silico screening may be performed using, for example, molecular dynamics simulations. Docking simulations may be performed (e.g., thermodynamic integration) between bound and unbound states to estimate the free energy of binding. A peptide design may be screened out, for instance, if the free energy of binding between the peptide and a protein in the protein is too specific, for example, as depicted in FIG. 11B. A peptide design may be screened out, for instance, if the free energy of binding between the peptide and a protein in the protein is too small in comparison to the thermal energy.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Numbered Embodiments

Embodiments contemplated herein include embodiments 1 to 260.

Embodiment 1. A system comprising: (a) a surface; (b) a peptide coupled to the surface, wherein the peptide comprises a binding site; and (c) at least three different biomolecules bound to the peptide at the binding site, wherein the at least three different biomolecules comprise at least three different proteins or at least five different biomolecules.

Embodiment 2. The system of embodiment 1, wherein the at least three different biomolecules are bound to a single instance of the peptide.

Embodiment 3. The system of embodiment 1, wherein the at least three different biomolecules are individually bound to different instances of the peptide.

Embodiment 4. The system of any one of embodiments 1-3, wherein the peptide comprises a synthetic sequence.

Embodiment 5. The system of any one of embodiments 1-4, wherein the peptide comprises one or more non-natural amino acids.

Embodiment 6. The system of any one of embodiments 1-5, wherein the peptide comprises a variant of a given peptide configured to bind to a different biomolecule in the at least three different proteins.

Embodiment 7. The system of any one of embodiments 1-6, wherein the peptide comprises a portion of a given binding site of the given peptide.

Embodiment 8. The system of any one of embodiments 1-7, wherein the peptide comprises a mutation of the given peptide such that the peptide comprises a modified form of the given binding site of the given peptide.

Embodiment 9. The system of embodiment 8, wherein the modified form comprises a different geometry than the given binding site of the given peptide

Embodiment 10. The system of embodiment 8 or 9, wherein the modified form comprises different vibrational modes, as measured by IR spectra or Raman spectroscopy.

Embodiment 11. The system of any one of embodiments 6-10, wherein the peptide comprises less specificity than a given binding site of the given peptide.

Embodiment 12. The system of any one of embodiments 1-11, wherein a first free energy of binding between the peptide and a first protein in the at least three different proteins is substantially equal to a second free energy of binding between the peptide and a second protein in the at least three different proteins, as measured in an aqueous solution comprising an ITC buffer.

Embodiment 13. The system of embodiment 12, wherein the first free energy of binding between the peptide and the first protein in the at least three different proteins is substantially equal to a third free energy of binding between the peptide and a third protein in the at least three different proteins, as measured in the aqueous solution comprising the ITC buffer.

Embodiment 14. The system of any one of embodiments 1-13, wherein a first equilibrium constant of binding between the peptide and a first protein in the at least three different proteins is substantially equal to a second equilibrium constant of binding between the peptide and a second protein in the at least three different proteins, as measured in an aqueous solution comprising an ITCbuffer.

Embodiment 15. The system of embodiment 14, wherein the first equilibrium constant of binding between the peptide and the first protein in the at least three different proteins is substantially equal to a third equilibrium constant of binding between the peptide and a third protein in the at least three different proteins, as measured in an aqueous solution comprising an ITC buffer.

Embodiment 16. The system of any one of embodiments 1-15, wherein the at least three different proteins comprise at least 4, 5, 6, 7, 8, 9, or 10 different proteins

Embodiment 17. The system of any one of embodiments 1-16, wherein the at least three different proteins comprise at least 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 different proteins.

Embodiment 18. The system of any one of embodiments 1-17, wherein a first amount of a first protein in the at least three different proteins bound to the peptide and a second amount of a second protein in the at least three different proteins bound to the peptide are within 1 magnitude of each other.

Embodiment 19. The system of embodiment 18, wherein the first amount of the first protein in the at least three different proteins bound to the peptide and a third amount of a third protein in the at least three different proteins bound to the peptide are within 1 magnitude of each other.

Embodiment 20. The system of any one of embodiments 1-19, wherein the peptide is coupled to the surface at a density of at least about 1 peptide per 5 nanometers, 1 peptide per 50 nanometers squared, or 1 peptide per 500 nanometers.

Embodiment 21. The system of any one of embodiments 1-20, wherein the peptide comprises at least 20 amino acids, at most 40 amino acids, or both.

Embodiment 22. The system of any one of embodiments 1-21, wherein the peptide comprises a plurality of modular units.

Embodiment 23. The system of any one of embodiments 1-22, wherein the peptide comprises a substantially linear domain.

Embodiment 24. The system of any one of embodiments 1-23, wherein the at least three different proteins are specifically bound to the peptide.

Embodiment 25. The system of any one of embodiments 1-24, wherein the surface further comprises a plurality of peptides coupled thereto, wherein each peptide in the plurality of peptides is configured to bind to at least three different proteins.

Embodiment 26. The system of embodiment 25, wherein a first peptide in the plurality of peptides is configured to bind to a first set of at least three different proteins, and a second peptide in the plurality of peptides is configured to bind to a second set of at least three different proteins, wherein the first set and the second set are different.

Embodiment 27. The system of any one of embodiments 1-26, wherein the at least three different proteins comprise the same epitope.

Embodiment 28. The system of any one of embodiments 1-27, wherein the at least three different proteins comprise at least two proteoforms expressed at least partially from the same locus of exons.

Embodiment 29. The system of any one of embodiments 1-28, wherein the system further comprises a plurality of biomolecules adsorbed on the surface, wherein the plurality of biomolecules comprises the at least three different proteins.

Embodiment 30. The system of embodiment 29, wherein the plurality of biomolecules comprises at least about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 proteins not specifically bound to the peptide.

Embodiment 31. The system of embodiment 29 or 30, wherein the plurality of biomolecules comprises a dynamic range of at least about 5, 6, 7, 8, 9, 10, 11, 12, 13, 14, 15, 16, 17, 18, 19, or 20.

Embodiment 32. The system of any one of embodiments 1-31, wherein the surface is provided in a solution with at least about 0.05, 0.1, 0.2, 0.3, 0.4, or 0.5 cm² in surface area of the surface per μL of the solution; or wherein the surface is provided in the solution with at most about 0.1, 0.2, 0.3, 0.4, 0.5, or 1.0 in surface area of the surface per μL of the solution; or both.

Embodiment 33. The system of any one of embodiments 1-31, wherein the surface is provided in a solution with at least about 1, 2, 3, 4, 5, or 10 μg in mass of a substance comprising the surface per μL of the solution; or wherein the surface is provided in the solution with at most about 2, 3, 4, 5, or 20 μg in mass of the substance comprising the surface per μL of the solution; or both.

Embodiment 34. The system of any one of embodiments 1-31, wherein the surface is provided in a solution with at least about 1, 2, 3, 4, 5, or 10 μg in mass of a substance comprising the surface per mg of the solution; or wherein the surface is provided in the solution with at most about 2, 3, 4, 5, or 20 μg in mass of the substance comprising the surface per mg of the solution; or both.

Embodiment 35. The system of any one of embodiments 1-34, wherein the surface is disposed in a porous material.

Embodiment 36. The system of any one of embodiments 1-35, wherein the surface is disposed on a particle.

Embodiment 37. The system of embodiment 36, wherein the particle comprises a paramagnetic material.

Embodiment 37. The system of embodiment 36 or 37, wherein the particle comprises a core comprising the paramagnetic material.

Embodiment 38. The system of embodiment 37, wherein the paramagnetic material comprises iron oxide.

Embodiment 39. The system of any one of embodiments 36-38, wherein the particle comprises a nanoparticle or a microparticle.

Embodiment 40. The system of any one of embodiments 36-39, wherein the particle forms a biomolecule corona comprising a plurality of biomolecules.

Embodiment 41. The system of any one of embodiments 29-40, wherein the plurality of biomolecules is non-specifically bound to the surface.

Embodiment 42. The system of any one of embodiments 29-41, wherein the plurality of biomolecules is captured on the surface such that a ratio of abundance between at least two biomolecules in the plurality of biomolecules in solution is changed for the at least two biomolecules in the plurality of biomolecules captured on the surface.

Embodiment 43. The system of any one of embodiments 29-42, wherein a plurality of biomolecules is increased in visibility in a downstream assay.

Embodiment 44. The system of embodiment 43, wherein the visibility of a biomolecule in the plurality of biomolecules is measurable by an intensity as measured by mass spectrometry.

Embodiment 45. The system of any one of embodiments 1-44, wherein the peptide is covalently coupled to the surface.

Embodiment 46. The system of any one of embodiments 1-45, wherein the surface comprises a silica layer comprising the surface.

Embodiment 47. The system of embodiment 46, wherein the silica layer comprises a linker that covalently couples the peptide to the surface

Embodiment 48. The system of embodiment 47, wherein the linker comprises at least one of: a silanization chemistry, a PEGylation chemistry, a maleimide chemistry, a succinimidyl ester chemistry, an isothiocyanate chemistry, a click chemistry, a thiol chemistry, a trans-4-(Maleimidomethyl)cyclohexanecarboxylic Acid-NHS (SMCC) chemistry, a biorthogonal chemistry, or any combination thereof.

Embodiment 49. The system of embodiment 47 or 48, wherein the linker comprises a density of at least about 1 linker per 1 nanometer squared, 1 linker per 3 nanometers squared, or 1 linker per 30 nanometers squared on the surface.

Embodiment 50. The system of any one of embodiments 47-49, wherein the linker comprises a length of at least about 8, 16, 32, 64, 128, 256, 512, 1024 covalent bonds from the surface to the peptide.

Embodiment 51. The system of any one of embodiments 47-49, wherein the linker comprises dimensions sufficient for the peptide to extend at least about 2 nm away from the surface.

Embodiment 52. The system of any one of embodiments 1-51, wherein the surface comprises a surface zeta potential of about 13-35 mV.

Embodiment 53. The system of any one of embodiments 1-52, wherein the surface comprises a surface zeta potential of at least about 0.01 mV in magnitude.

Embodiment 54. The system of any one of embodiments 1-53, wherein the surface comprises a surface zeta potential of at least about 0.1 mV in magnitude, wherein the surface comprises a surface zeta potential of at least about 1 mV in magnitude.

Embodiment 55. The system of any one of embodiments 1-49, wherein the surface comprises a surface zeta potential of at least about 10, 100, or 1000 mV in magnitude.

Embodiment 56. The system of any one of embodiments 1-55, wherein the surface comprises a hydrophobic surface or a hydrophilic surface.

Embodiment 57. The system of any one of embodiments 1-56, wherein the surface comprises an elemental carbon fraction of about 10-43% as measured by XPS.

Embodiment 58. The system of any one of embodiments 1-57, wherein the surface comprises an elemental oxygen fraction of about 40-60% as measured by XPS.

Embodiment 59. The system of any one of embodiments 1-58, wherein the surface comprises an elemental nitrogen fraction of about 1-4% as measured by XPS.

Embodiment 60. The system of any one of embodiments 1-59, wherein the surface comprises an elemental sulfur composition of about 0.5-2% as measured by XPS.

Embodiment 61. The system of any one of embodiments 1-60, wherein the surface comprises an elemental silicon composition of about 24-31% as measured by XPS.

Embodiment 62. The system of any one of embodiments 1-55, wherein the surface comprises an elemental iron composition of about 0% as measured by XPS.

Embodiment 63. The system of any one of embodiments 57-62, wherein the XPS is performed up to a depth of at most about 10 nm.

Embodiment 64. The system of any one of embodiments 57-63, wherein the XPS is performed up to a depth of at most about 1 nm or 5 nm.

Embodiment 65. A system comprising an N number of surfaces, wherein an n-th surface in the N number of surface comprises: (a) an n-th peptide coupled to the n-th surface; and (b) an n-th plurality of distinct biomolecules non-specifically bound to the n-th peptide, wherein N is at least 2, and n ranges from 1 to N.

Embodiment 66. The system of embodiment 65, wherein N is at least 3, 4, 5, 6, 7, 8, 9, or 10.

Embodiment 67. The system of embodiment 65 or 66, wherein at least one n-th plurality of distinct biomolecules comprises at least 3, 4, 5, 6, 7, 8, 9, or 10 different biomolecules.

Embodiment 68. The system of embodiment 67, wherein at least 2, 3, 4, 5, 6, 7, 8, 9, or 10 n-th plurality of distinct biomolecules each comprise at least 3, 4, 5, 6, 7, 8, 9, or 10 different biomolecules.

Embodiment 69. The system of any one of embodiments 65-68, wherein a first amount of a first biomolecule in at least one n-th plurality of distinct biomolecules and a second amount of a second biomolecule in the at least one n-th plurality of distinct biomolecules are within 1 magnitude of each other.

Embodiment 70. The system of embodiments 69, wherein the first amount of the first biomolecule in the at least one n-th plurality of distinct biomolecules and a third amount of a third biomolecule in the at least one n-th plurality of distinct biomolecules are within 1 magnitude of each other.

Embodiment 71. The system of any one of embodiments 65-70, wherein at least one n-th peptide comprises a variant of a given peptide configured to bind to a different biomolecule in the at least three different proteins.

Embodiment 72. The system of any one of embodiments 65-71, wherein the at least one n-th peptide comprises a portion of a given binding site of the given peptide.

Embodiment 73. The system of any one of embodiments 65-72, wherein the at least one n-th peptide comprises a mutation of the given peptide such that the at least one n-th peptide comprises a modified form of the given binding site of the given peptide.

Embodiment 74. The system of embodiment 73, wherein the modified form comprises a different geometry than the given binding site of the given peptide

Embodiment 75. The system of embodiment 73 or 74, wherein the modified form comprises different vibrational modes, as measured by IR spectra or Raman spectroscopy.

Embodiment 76. The system of any one of embodiments 71-75, wherein the at least one n-th peptide comprises less specificity than a given binding site of the given peptide.

Embodiment 77. The system of any one of embodiments 65-76, wherein a first free energy of binding between the at least one n-th peptide and a first biomolecule in at least one n-th plurality of distinct biomolecules and a second free energy of binding between the at least one n-th peptide and a second biomolecule in the at least one n-th plurality of distinct biomolecules are within 1 magnitude of each other.

Embodiment 78. The system of embodiment 77, wherein the first free energy of binding between the at least one n-th peptide and the first biomolecule in at least one n-th plurality of distinct biomolecules and a third free energy of binding between the at least one n-th peptide and a third biomolecule in the at least one n-th plurality of distinct biomolecules are within 1 magnitude of each other.

Embodiment 79. The system of any one of embodiments 65-78, wherein a first equilibrium constant of binding between the at least one n-th peptide and a first biomolecule in at least one n-th plurality of distinct biomolecules is substantially equal to a second equilibrium constant of binding between the at least one n-th peptide and a second biomolecule in the at least one n-th plurality of distinct biomolecules, as measured in an aqueous solution comprising an ITC buffer.

Embodiment 80. The system of embodiment 79, wherein the first free energy of binding between the at least one n-th peptide and the first biomolecule in at least one n-th plurality of distinct biomolecules is substantially equal to a third free energy of binding between the at least one n-th peptide and a third biomolecule in the at least one n-th plurality of distinct biomolecules, as measured in an aqueous solution comprising an ITC buffer.

Embodiment 81. The system of any one of embodiments 65-80, wherein the at least one n-th peptide is coupled to the surface at a density of at least about 1 peptide per 50 nanometers squared.

Embodiment 82. The system of any one of embodiments 65-80, wherein the peptide is coupled to the surface at a density of at most about 1 peptide per 5 nanometers squared.

Embodiment 83. The system of any one of embodiments 65-80, wherein the peptide is coupled to the surface at a density of at most about 1 peptide per 500 nanometers squared.

Embodiment 84. The system of any one of embodiments 65-83, wherein the at least one n-th peptide comprises at least 20 amino acids, at most 40 amino acids, or both.

Embodiment 85. The system of any one of embodiments 65-84, wherein the at least one n-th peptide comprises a substantially linear domain.

Embodiment 86. The system of any one of embodiments 65-85, wherein at least one n-th surface further comprises a plurality of peptides coupled thereto, wherein each peptide in the plurality of peptides is configured to bind to at least three different proteins.

Embodiment 87. The system of any one of embodiments 65-86, wherein the at least three different proteins in the at least one n-th plurality of distinct biomolecules comprise the same epitope.

Embodiment 88. The system of embodiment 87, wherein the at least three different proteins comprise at least two proteoforms expressed at least partially from the same locus of exons.

Embodiment 89. A method comprising: (a) contacting a biological sample with a surface in the system of any one of embodiments 1-88 to bind a plurality of biomolecules to a peptide coupled to the surface; (b) releasing the plurality of biomolecules or a portion thereof from the surface; (c) identifying at least the plurality of biomolecules or the portion thereof, wherein the plurality of biomolecules or the portion thereof comprises one or more biomolecules in the at least three different proteins in the biological sample.

Embodiment 90. The method of embodiment 89, wherein the identifying comprises performing mass spectrometry or nucleic acid sequencing on the plurality of biomolecules or the portion thereof released in (b).

Embodiment 91. A composition comprising a particle having on its surface less than 5 distinct types of binding molecules, said binding molecules having binding specificity for a subset of proteins in a sample, wherein said subset of proteins comprises about 10-500 proteins.

Embodiment 92. The composition of embodiment 91, wherein said binding molecule comprises a peptide comprising at least 20 amino acids.

Embodiment 93. The composition of embodiment 92, wherein said peptide comprises at most 40 amino acids.

Embodiment 94. The composition of embodiment 91, wherein said particle comprises a single type of binding molecule.

Embodiment 95. The composition of embodiment 91, wherein said particle comprises 2-5 distinct types of binding molecules.

Embodiment 96. The composition of any one of embodiments 91-95, wherein said peptide comprises between 7 and 20 amino acids.

Embodiment 97. The composition of embodiment 96, wherein said peptide comprises between 7 and 15 amino acids.

Embodiment 98. The composition of embodiment 97, wherein said peptide comprises between 8 and 12 amino acids.

Embodiment 99. The composition of any one of embodiments 91-98, wherein said peptide comprises amino acids selected from the group consisting of alanine, arginine, aspartic acid, glutamic acid, glutamine, glycine, histidine, isoleucine, leucine, lysine, proline, serine, asparagine, threonine, and valine.

Embodiment 100. The composition of any one of embodiments 91-99, wherein said peptide is free of one or more of cysteine, methionine, tryptophan, tyrosine, and phenylalanine.

Embodiment 101. The composition of any one of embodiments 91-100, wherein said peptide comprises a non-proteinogenic amino acid.

Embodiment 102. The composition of embodiment 101, wherein said non-proteinogenic amino acid is a chemically modified proteinogenic amino acid.

Embodiment 103. The composition of embodiment 102, wherein said chemical modification comprises acylation, alkylation, amidation, deamidation, carbamylation, carbonylation, carboxylation, decarboxylation, citrullination, flavination, glycosylation, halogenation, hydroxylation, nitrosylation, oxidation, phosphorylation, prenylation, racemization, reduction, succinylation, sulfation, or any combination thereof.

Embodiment 104. The composition of any one of embodiments 91-103, wherein said peptide comprises an isoelectric point (pI) of between 5 and 10.

Embodiment 105. The composition of any one of embodiments 91-104, wherein said particle comprises an isoelectric point of within 0.5 of said peptide.

Embodiment 106. The composition of any one of embodiments 91-105, wherein a conformation of said peptide comprises a dependence on pH, temperature, ionic strength, dielectric constant, viscosity, or any combination thereof.

Embodiment 107. The composition of any one of embodiments 91-106, wherein said peptide is coupled to said surface of said particle.

Embodiment 108. The composition of embodiment 107, wherein said peptide is coupled directly to said surface of said particle.

Embodiment 109. The composition of embodiment 108, wherein a C-terminus of said peptide is coupled to said surface of said particle.

Embodiment 110. The composition of embodiment 108, wherein an N-terminus of said peptide is coupled to said surface of said particle.

Embodiment 111. The composition of embodiment 108, wherein an internal amino acid of said peptide is coupled to said surface of said particle.

Embodiment 112. The composition of embodiment 107, wherein said peptide is coupled to a chemical linker coupled to said surface of said particle.

Embodiment 113. The composition of embodiment 112, wherein a C-terminus of said peptide is coupled to said chemical linker.

Embodiment 114. The composition of embodiment 112, wherein an N-terminus of said peptide is coupled to said chemical linker.

Embodiment 115. The composition of embodiment 112, wherein an internal amino acid of said peptide is coupled to said chemical linker.

Embodiment 116. The composition of embodiment 114 or 115, wherein said N-terminus or said internal amino acid comprises an amide bond to said chemical linker.

Embodiment 117. The composition of any one of embodiments 112-116, wherein said chemical linker comprises a maleimide group.

Embodiment 118. The composition of any one of embodiments 91-117, wherein said particle comprises 2 to 5 distinct types of peptides.

Embodiment 119. The composition of embodiment 118, wherein said 2 to 5 distinct peptides comprise at least two peptides having different lengths.

Embodiment 120. The composition of embodiment 118 or 119, wherein said 2 to 5 distinct peptides have substantially similar isoelectric points.

Embodiment 121. The composition of embodiment 118 or 119, wherein said 2 to 5 distinct peptides have at least two different isoelectric points.

Embodiment 122. The composition of any one of embodiments 118-121, wherein said particle comprises specific molar ratios of said 2 to 5 distinct peptides.

Embodiment 123. The composition of any one of embodiments 91-122, wherein said sample is a biological sample.

Embodiment 124. The composition of embodiment 123, wherein said biological sample comprises whole blood, plasma, buffy coat, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 125. The composition of embodiment 123 or 124, wherein said biological sample comprises plasma or serum.

Embodiment 126. The composition of any one of embodiments 123-125, wherein said biological sample comprises a liquid.

Embodiment 127. The composition of any one of embodiments 91-126, wherein said biological sample comprises greater than 1000 types of proteins.

Embodiment 128. The composition of any one of embodiments 91-127, wherein said biological sample comprises a protein concentration dynamic range of at least 8 orders of magnitude.

Embodiment 129. The composition of any one of embodiments 91-128, wherein said subset of proteins comprises 60-200 proteins.

Embodiment 130. The composition of any one of embodiments 91-129, wherein said subset of proteins comprises 80-160 proteins.

Embodiment 131. The composition of any one of embodiments 91-130, wherein said subset of proteins comprises 100-140 proteins.

Embodiment 132. The composition of any one of embodiments 91-131, wherein said subset of proteins comprises 10-50 proteins.

Embodiment 133. The composition of any one of embodiments 91-132, wherein said subset of proteins comprises 10-30 proteins.

Embodiment 134. The composition of any one of embodiments 91-133, wherein said subset of proteins comprises a dynamic range of at least 4 orders of magnitude.

Embodiment 135. The composition of any one of embodiments 91-134, wherein said subset of proteins comprises a dynamic range of at least 6 orders of magnitude.

Embodiment 136. The composition of any one of embodiments 91-135, wherein said subset of proteins comprises a high abundance protein.

Embodiment 137. The composition of embodiment 136, wherein said high abundance protein comprises at least 0.1% of the protein mass of said sample.

Embodiment 138. The composition of embodiment 136 or 137, wherein said high abundance protein comprises a concentration of at least 1 micromolar (μM).

Embodiment 139. The composition of any one of embodiments 91-138, wherein said composition comprises a plurality of particles comprising said particle.

Embodiment 140. The composition of embodiment 139, wherein individual particles of said plurality of particles comprise the same binding molecules.

Embodiment 141. The composition of any one of embodiments 91-140, wherein said binding specificity comprises at most 1 micromolar (μM) dissociation constants.

Embodiment 142. The composition of any one of embodiments 91-141, wherein said binding specificity comprises at most 1 nanomolar (nM) dissociation constants.

Embodiment 143. The composition of any one of embodiments 91-142, wherein said binding molecules comprise an average spacing of at least 4 nanometers (nm) on said surface of said particle.

Embodiment 144. The composition of any one of embodiments 91-143, wherein said binding molecules comprise an average spacing of at least 10 nanometers (nm) on said surface of said particle.

Embodiment 145. A method for enriching a subset of proteins in a sample, comprising: (a) contacting said sample with a composition comprising a particle, said particle having on its surface less than 5 distinct types of binding molecules which have binding specificity for said subset of proteins in said sample; and (b) capturing said subset of proteins using said particle, to thereby enrich said subset of proteins, wherein said subset of proteins comprises about 10-500 proteins.

Embodiment 146. The method of embodiment 145, wherein said contacting comprises adding between 100 picomolar (pM) and 100 nanomolar (nM) of said particle to said sample.

Embodiment 147. The method of either of embodiments 145 or 146, wherein said capturing comprises incubating said particle in said sample for at least 1 hour.

Embodiment 148. The method of any one of embodiments 145-147, wherein said capturing comprises incubating said particle in said sample at a temperature of at least 37° C.

Embodiment 149. The method of any one of embodiments 145-148, wherein said capturing comprises collecting at least 10⁻⁹ mg of said subset of said proteins per square millimeter (mm²) of surface area of said particle.

Embodiment 150. The method of any one of embodiments 145-149, wherein said enriching comprises narrowing a dynamic range of said subset of proteins.

Embodiment 151. The method of embodiment 145, wherein said particle comprises a single type of binding molecule.

Embodiment 152. The method of embodiment 145, wherein said particle comprises 2-5 distinct types of binding molecules.

Embodiment 153. The method of embodiment 145, wherein said binding molecules comprise a peptide.

Embodiment 154. The method of embodiment 153, wherein said peptide comprises between 7 and 20 amino acids.

Embodiment 155. The method of either of embodiments 153 or 154, wherein said peptide is free of one or more of cysteine, methionine, tryptophan, tyrosine, and phenylalanine.

Embodiment 156. The method of any one of embodiments 153-155, wherein said peptide comprises an isoelectric point (pI) of between 5 and 10.

Embodiment 157. The method of any one of embodiments 153-156, wherein said particle comprises an isoelectric point of within 0.5 of said peptide.

Embodiment 158. The method of any one of embodiments 153-157, wherein a conformation of said peptide comprises a dependence on pH, temperature, ionic strength, dielectric constant, viscosity, or any combination thereof.

Embodiment 159. The method of any one of embodiments 153-158, wherein said peptide comprises 2 to 5 distinct types of peptides.

Embodiment 160. The method of embodiment 159, wherein said 2 to 5 distinct peptides comprise at least two different isoelectric points.

Embodiment 161. The method of embodiment 159 or 160, wherein said particle comprises specific molar ratios of said 2 to 5 distinct peptides.

Embodiment 162. The method of any one of embodiment 153-161, wherein said sample is a biological sample.

Embodiment 163. The method of embodiment 162, wherein said biological sample comprises whole blood, plasma, buffy coat, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 164. The method of embodiment 163, wherein said biological sample comprises plasma or serum.

Embodiment 165. The method of any one of embodiments 162-164, wherein said biological sample comprises a liquid.

Embodiment 166. The method of any one of embodiments 162-165, wherein said biological sample comprises greater than 1000 types of proteins.

Embodiment 167. The method of embodiment 166, wherein said biological sample comprises greater than 5000 types of proteins.

Embodiment 168. The method of any one of embodiments 153-167, wherein said subset of proteins comprises 60-200 proteins.

Embodiment 169. The method of any one of embodiments 153-168, wherein said subset of proteins comprises 10-50 proteins.

Embodiment 170. The method of any one of embodiments 153-169, wherein said subset of proteins comprises a high abundance protein.

Embodiment 171. The method of any one of embodiments 153-170, wherein said composition comprises a plurality of particles comprising said particle.

Embodiment 172. A composition for enriching proteins from a sample, said composition comprising at least two distinct particle types each comprising surface bound binding molecules having binding specificity for a different set of proteins of said sample, wherein a set of proteins of a first particle type and a second set of proteins of a second particle type have between 10%-85% overlap.

Embodiment 173. The composition of embodiment 172, wherein said first set of proteins and said second set of proteins comprise at most 70% overlap.

Embodiment 174. The composition of embodiment 172 or 173, wherein said first set of proteins or said second set of proteins has a dynamic range of at least 4 orders of magnitude.

Embodiment 175. The composition of embodiment 174, wherein said dynamic range is at most 6 orders of magnitude.

Embodiment 176. The composition of any one of embodiments 172-175, wherein said surface bound binding molecules comprise no more than 5 distinct types of binding molecules.

Embodiment 177. The composition of any one of embodiments 172-176, wherein said surface bound binding molecules comprise peptides.

Embodiment 178. The composition of embodiment 177, wherein said peptides comprise between 7 and 20 amino acids.

Embodiment 179. The composition of embodiment 177 or 178, wherein said peptides comprise between 7 and 15 amino acids.

Embodiment 180. The composition of any one of embodiments 177-179, wherein said peptides comprise between 8 and 12 amino acids.

Embodiment 181. The composition of any one of embodiments 177-180, wherein said peptides are free of one or more of cysteine, methionine, tryptophan, tyrosine, and phenylalanine.

Embodiment 182. The composition of any one of embodiments 177-181, wherein said peptides comprise a non-proteinogenic amino acid.

Embodiment 183. The composition of any one of embodiments 177-182, wherein said peptides comprise isoelectric points between 5 and 10.

Embodiment 184. The composition of any one of embodiments 177-183, wherein said peptides comprise 2 to 5 types of peptides per particle type of said at least two distinct particle types.

Embodiment 185. The composition of embodiment 184, wherein said 2 to 5 distinct peptides comprise substantially similar isoelectric points.

Embodiment 186. The composition of embodiment 184, wherein said 2 to 5 distinct peptides comprise at least two different isoelectric points.

Embodiment 187. The composition of any one of embodiments 172-186, wherein said given sets of proteins each comprise between 5 and 500 proteins.

Embodiment 188. The composition of any one of embodiments 172-187, wherein said given sets of proteins each comprise between 10 and 500 proteins.

Embodiment 189. The composition of any one of embodiments 172-188, wherein said given sets of proteins each comprise between 10 and 200 proteins.

Embodiment 190. The composition of any one of embodiments 172-189, wherein said given sets of proteins each comprise between 10 and 50 proteins.

Embodiment 191. The composition of any one of embodiments 172-190, wherein said given sets of proteins each comprise between 10 and 30 proteins.

Embodiment 192. The composition of any one of embodiments 174-191, wherein dynamic ranges of first set of proteins and said second set of proteins substantially overlap with each other.

Embodiment 193. A method for assaying a sample, comprising: (a) contacting said sample with a composition comprising a first surface-modified particle configured to enrich for a first subset of proteins in said sample, and a second surface-modified particle configured to enrich for a second subset of proteins in said sample; and (b) enriching for said first subset and said second subset of proteins in said sample using said composition, wherein said first subset of proteins and said second subset of proteins each comprises no more than three proteins that have a concentration of greater than or equal to about 10 μg/mL in said sample, and differ from each other in at least one of such proteins.

Embodiment 194. The method of embodiment 193, wherein said composition comprises between 100 picomolar (pM) and 100 nanomolar (nM) of said first surface-modified particle and said second surface-modified particle.

Embodiment 195. The method of either of embodiments 193 or 194, wherein said enriching comprises incubating said first surface-modified particle and said second surface-modified particle in said sample for at least 1 hour.

Embodiment 196. The method of any one of embodiments 193-195, wherein said enriching comprises incubating said first surface-modified particle and said second surface-modified particle in said sample at a temperature of at least 37° C.

Embodiment 197. The method of any one of embodiments 193-196, wherein said enriching comprises collecting at least 10⁻⁹ mg of said subsets of proteins per square millimeter (mm²) of surface area of said first surface-modified particle and/or said second surface-modified particle.

Embodiment 198. The method of any one of embodiments 193-197, wherein said first surface-modified particle and said second surface-modified particle each comprise between 1 and 5 distinct types of surface bound binding molecules.

Embodiment 199. The method of any one of embodiments 193-198, wherein said first surface-modified particle and said second surface-modified particle each comprise between 2 and 5 distinct types of surface bound binding molecules.

Embodiment 200. The method of any one of embodiments 193-199, wherein said first surface-modified particle and said second surface-modified particle each comprise at most 2 distinct types of surface bound binding molecules.

Embodiment 201. The method of any one of embodiments 193-200, wherein said first surface-modified particle or said second surface-modified particle comprises exactly 1 type of surface bound binding molecule.

Embodiment 202. The method of any one of embodiments 193-201, wherein said first surface-modified particle or said second surface-modified particle comprises surface-bound peptides.

Embodiment 203. The method of embodiment 202, wherein said surface-bound peptides comprise between 7 and 20 amino acids.

Embodiment 204. The method of either of embodiments 202 or 03, wherein said surface-bound peptides are free of one or more of cysteine, methionine, tryptophan, tyrosine, and phenylalanine.

Embodiment 205. The method of any one of embodiments 202-204, wherein said surface-bound peptides comprise isoelectric points (pI) between 5 and 10.

Embodiment 206. The method of any one of embodiments 202-205, wherein said first surface-modified particle and said second surface-modified particle comprise different peptides.

Embodiment 207. The method of embodiment 206, wherein said first surface-modified particle and said second surface-modified particle comprise different isoelectric points.

Embodiment 208. The method of any one of embodiments 202-207, wherein said first surface-modified particle and said second surface-modified particle each comprise between 1 and 5 different peptides.

Embodiment 209. The method of any one of embodiments 193-208, wherein said sample is a biological sample.

Embodiment 210. The method of embodiment 209, wherein said biological sample comprises whole blood, plasma, buffy coat, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 211. The method of embodiment 210, wherein said biological sample comprises plasma or serum.

Embodiment 212. The method of any one of embodiments 209-211, wherein said biological sample comprises greater than 1000 types of proteins.

Embodiment 213. A composition comprising a plurality of particles having surfaces comprising oligopeptides having 7 or more amino acid residues, said oligopeptides having binding specificity for a subset of proteins in a sample, wherein said subset of proteins comprises less than or equal to about 500 proteins.

Embodiment 214. The composition of embodiment 213, wherein said oligopeptides comprise between 7 and 20 amino acid residues, at least 20 amino acid residues, or at most 40 amino acid residues.

Embodiment 215. The composition of embodiment 214 or 215, wherein said oligopeptides comprise between 7 and 15 amino acid residues.

Embodiment 216. The composition of any one of embodiments 213-216, wherein said binding specificity is at most 100 micromolar (μM).

Embodiment 217. The composition of any one of embodiments 213-217, wherein said binding specificity is at least 1 micromolar (μM).

Embodiment 218. The composition of any one of embodiments 213-218, wherein said binding specificity has a pH dependence.

Embodiment 219. The composition of any one of embodiments 213-218, wherein said plurality of particles comprises at least a first particle having on its surface a first set of oligopeptides and a second particle having on its surface a second set of oligopeptides different from said first set of oligopeptides.

Embodiment 220. The composition of embodiment 219, wherein an isoelectric point of said first particle differs from an isoelectric point of said second particle.

Embodiment 221. The composition of embodiment 219 or 220, wherein said first set and second set of oligopeptides have binding specificity for a first subset of proteins and a second subset of proteins in said sample, respectively, and wherein said first subset of proteins is different from said second subset of proteins.

Embodiment 222. The composition of embodiment 221, wherein said first subset of proteins and said second subset of proteins comprise at most 85% overlap.

Embodiment 223. The composition of any one of embodiments 213-222, wherein said subset of proteins comprises a high abundance protein.

Embodiment 224. The composition of embodiment 223, wherein said first subset of proteins and said second subset of proteins comprise at most 85% overlap between their high abundance proteins.

Embodiment 225. The composition of any one of embodiments 213-224, wherein said subset of proteins comprises less than or equal to about 200 proteins.

Embodiment 226. A method for enriching a subset of proteins in a sample, comprising: (a) contacting said sample with a composition comprising a plurality of particles having surfaces comprising oligopeptides having 7 or more amino acid residues, said oligopeptides having binding specificity for said subset of proteins in said sample; and (b) capturing said subset of proteins using said plurality of particles to thereby enrich said subset of proteins, wherein said subset of proteins comprises less than or equal to about 500 proteins.

Embodiment 227. The method of embodiment 226, wherein said contacting comprises adding between 100 picomolar (pM) and 100 nanomolar (nM) of said plurality of particles to said sample.

Embodiment 228. The method of either of embodiments 226 or 227, wherein said capturing comprises incubating said plurality of particles in said sample for at least 1 hour.

Embodiment 229. The method of any one of embodiments 226-228, wherein said capturing comprises incubating said plurality of particles in said sample at a temperature of at least 37° C.

Embodiment 230. The method of any one of embodiments 226-229, wherein said capturing comprises collecting at least 10⁻⁹ mg of said subset of said proteins per square millimeter (mm²) of surface area of said plurality of particles.

Embodiment 231. The method of any one of embodiments 226-230, wherein said oligopeptides comprise between 1 and 5 distinct types of oligopeptides.

Embodiment 232. The method of any one of embodiments 226-231, wherein said oligopeptides comprise between 7 and 20 amino acids.

Embodiment 233. The method of any one of embodiments 226-232, wherein said oligopeptides are free of one or more of cysteine, methionine, tryptophan, tyrosine, and phenylalanine.

Embodiment 234. The method of any one of embodiments 226-233, wherein said sample is a biological sample.

Embodiment 235. The method of embodiment 234, wherein said biological sample comprises plasma or serum.

Embodiment 236. The method of any one of embodiments 226-235, wherein said subset of proteins comprises less than about 200 proteins.

Embodiment 237. The method of any one of embodiments 226-235, wherein said subset of proteins comprises greater than about 60 proteins.

Embodiment 238. The method of any one of embodiments 226-235, wherein said subset of proteins comprises about 10 to about 100 proteins.

Embodiment 239. The method of embodiment 238, wherein said subset of proteins comprises about 10 to about 50 proteins.

Embodiment 240. A surface modified particle comprising one or more binding molecules attached to a surface thereof, said one or more binding molecules having a plurality of segments each of which is independently addressable.

Embodiment 241. The surface modified particle of embodiment 240, wherein individual segments of said plurality of segments are separated from one another by a linking element.

Embodiment 242. The surface modified particle of embodiment 241, wherein said linking element is not a peptide-derived linking element.

Embodiment 243. The surface modified particle of any one of embodiments 240-242, wherein individual segments of at least a subset of said plurality of segments are interchangeable.

Embodiment 244. The surface modified particle of any one of embodiments 240-243, wherein individual segments of said plurality of segments are capable of being removed, modified, or substituted.

Embodiment 245. The surface modified particle of any one of embodiments 240-244, wherein said one or more binding molecules comprise polymers, peptides, or a combination thereof.

Embodiment 246. The surface modified particle of embodiment 240, wherein said polymers are biodegradable.

Embodiment 247. The surface modified particle of embodiment 245 or 246, wherein said peptides comprise oligopeptides having about 7-20 amino acid residues.

Embodiment 248. The surface modified particle of any one of embodiments 240-247, wherein said plurality of segments comprise oligopeptides having about 1-6 amino acid residues.

Embodiment 249. The surface modified particle of any one of embodiments 240-248, wherein said particle is a nanoparticle having a diameter between 10 nanometers (nm) and 500 nm.

Embodiment 250. A kit comprising a substance comprising a surface and a peptide coupled thereto, wherein the peptide comprises a binding site configured to bind to a set of biomolecules, wherein the set of biomolecules comprises at least three proteins or at least five biomolecules.

Embodiment 251. The kit of embodiment 250, further comprising a second substance comprising a second surface and a second peptide coupled thereto, wherein the second peptide comprises a binding site configured to bind to a second set of biomolecules, wherein the second set of biomolecules is different from the first set of biomolecules.

Embodiment 252. The kit of embodiment 250 or 251, further comprising a proteolytic enzyme.

Embodiment 253. The kit of embodiment 252, wherein the proteolytic enzyme is trypsin or lysin.

Embodiment 254. The kit of any one of embodiments 250-253, further comprising a buffer.

Embodiment 255. The kit of embodiment 254, wherein the buffer comprises a lyse buffer.

Embodiment 256. The kit of any one of embodiments 250-255, wherein the substance is disposed in a chamber comprising a first cap, and the second substance is disposed in a chamber comprising a second cap, wherein the first cap and the second cap are different in color.

Embodiment 257. The kit of any one of embodiments 250-256, wherein the substance is disposed in a chamber comprising a first barcode, and the second substance is disposed in a chamber comprising a second barcode, wherein the first barcode and the second barcode are different.

Embodiment 258. The kit of any one of embodiments 250-257, comprising a container for containing the substance, wherein the container comprises a barcode.

Embodiment 259. A method comprising: (a) contacting a biological sample with the composition of any one of embodiments 91-144, 172-192, and 213-225 to capture a plurality of biomolecules to the composition; (b) releasing the plurality of biomolecules or a portion thereof from the composition; (c) identifying at least the plurality of biomolecules or the portion thereof, wherein the plurality of biomolecules or the portion thereof comprises one or more biomolecules in the at least three different proteins in the biological sample.

Embodiment 260. The method of embodiment 259, wherein the identifying comprises performing mass spectrometry or nucleic acid sequencing on the plurality of biomolecules or the portion thereof released in (b).

What is claimed is:

1. A system comprising:
(a) a surface;
(b) a peptide coupled to the surface, wherein the peptide comprises a binding site; and
(c) at least three different proteins bound to the peptide at the binding site.

2. The system of claim 1, wherein the at least three different proteins comprise at least 4, 5, 6, 7, 8, 9, or 10 different proteins.

3. The system of claim 1, wherein the at least three different proteins are bound to a single instance of the peptide.

4. The system of claim 1, wherein the at least three different proteins are individually bound to different instances of the peptide.

5. The system of claim 1, wherein a first amount of a first protein in the at least three different proteins bound to the peptide and a second amount of a second protein in the at least three different proteins bound to the peptide are within 1 magnitude of each other.

6. The system of claim 5, wherein the first amount of the first protein in the at least three different proteins bound to the peptide and a third amount of a third protein in the at least three different proteins bound to the peptide are within 1 magnitude of each other.

7. The system of claim 1, wherein the peptide is coupled to the surface at a density of at least about 1 peptide per 5 nanometers, 1 peptide per 50 nanometers squared, or 1 peptide per 50 nanometers squared.

8. The system of claim 1, wherein the peptide comprises at most about 40 amino acids.

9. The system of claim 8, wherein the peptide comprises at least about 20 amino acids.

10. The system of claim 1, wherein the peptide comprises a synthetic sequence.

11. The system of claim 1, wherein the peptide comprises non-natural amino acids.

12. The system of claim 1, wherein the surface further comprises a plurality of peptides coupled thereto, wherein each peptide in the plurality of peptides is configured to bind to at least three different proteins.

13. The system of claim 1, wherein at least a subset of proteins in the at least three different proteins comprise the same epitope.

14. The system of claim 1, wherein at least a subset of proteins in the at least three different proteins comprise at least two proteoforms expressed at least partially from the same locus of exons.

15. The system of claim 1, wherein the at least three different proteins are specifically bound to the peptide.

16. The system of claim 1, wherein the system further comprises a plurality of biomolecules adsorbed on the surface.

17. The system of claim 16, wherein the plurality of biomolecules comprises at least about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 proteins not specifically bound to the peptide.

18. The system of claim 16, wherein the plurality of biomolecules comprises a dynamic range of at least about 5, 6, 7, 8, 9, 10, 11, 12, 13, 14, 15, 16, 17, 18, 19, or 20.

19. The system of claim 1, wherein the surface is provided in a solution with at least about 0.05, 0.1, 0.2, 0.3, 0.4, or 0.5 cm² in surface area of the surface per μL of the solution.

20. The system of claim 1, wherein a plurality of biomolecules are captured on the surface such that a ratio of abundance between at least two biomolecules in the plurality of biomolecules in solution is changed for the at least two biomolecules in the plurality of biomolecules captured on the surface.

21. The system of claim 1, wherein a plurality of biomolecules is increased in visibility in a downstream assay.

22. The system of claim 21, wherein the visibility of a biomolecule in the plurality of biomolecules is measurable by an intensity as measured by mass spectrometry.

23. A system comprising an N number of surfaces, wherein an n-th surface in the N number of surface comprises:
(a) an n-th peptide coupled to the n-th surface; and
(b) an n-th plurality of distinct biomolecules non-specifically bound to the n-th peptide, wherein Nis at least 2, and n ranges from 1 to N.

24. The system of claim 23, wherein N is at least 3, 4, 5, 6, 7, 8, 9, or 10.

25. The system of claim 23, wherein at least one n-th plurality of distinct biomolecules comprises at least 3, 4, 5, 6, 7, 8, 9, or 10 different biomolecules.

26. The system of claim 25, wherein at least 2, 3, 4, 5, 6, 7, 8, 9, or 10 n-th plurality of distinct biomolecules each comprise at least 3, 4, 5, 6, 7, 8, 9, or 10 different biomolecules.

27. The system of claim any one of claims 23-26, wherein a first amount of a first biomolecule in at least one n-th plurality of distinct biomolecules and a second amount of a second biomolecule in the at least one n-th plurality of distinct biomolecules are within 1 magnitude of each other.

28. The system of claim 27, wherein the first amount of the first biomolecule in the at least one n-th plurality of distinct biomolecules and a third amount of a third biomolecule in the at least one n-th plurality of distinct biomolecules are within 1 magnitude of each other.

29. The system of claim 23, wherein the n-th plurality of distinct biomolecules comprises one or more proteins.

30. A system comprising:
(a) a surface;
(b) a peptide covalently coupled to the surface, wherein the peptide comprises a binding site; and
(c) at least five different biomolecules bound to the peptide at the binding site.

31. The system of claim 30, wherein the at least five different biomolecules comprise at least 6, 7, 8, 9, or 10 different proteins.

32. The system of claim 30, wherein a first amount of a first biomolecule in the at least five different biomolecules bound to the peptide and a second amount of a second biomolecule in the at least five different biomolecules bound to the peptide are within 1 magnitude of each other.

33. The system of claim 32, wherein the first amount of the first biomolecule in the at least five different biomolecules bound to the peptide and a third amount of a third biomolecule in the at least five different biomolecules bound to the peptide are within 1 magnitude of each other.

34. The system of claim 30, wherein the peptide is coupled to the surface at a density of at least about 1 peptide per 50 nanometers squared.

35. The system of claim 34, wherein the peptide comprises at most about 40 amino acids.

36. The system of claim 35, wherein the peptide comprises at least about 20 amino acids.

37. A method comprising:
(a) contacting a biological sample with a surface to bind a plurality of biomolecules to a peptide coupled to the surface, wherein the peptide is configured to bind to at least three different proteins;
(b) releasing the plurality of biomolecules or a portion thereof from the surface; and
(c) identifying at least the plurality of biomolecules or the portion thereof, wherein the plurality of biomolecules or the portion thereof comprises one or more biomolecules in the at least three different proteins in the biological sample.

38. The method of claim 37, wherein the identifying in (c) further comprising identifying the plurality of biomolecules, thereby determining interactions between at least a first portion of the three different proteins and at least a second portion of the plurality of biomolecules.

39. The method of claim 38, wherein the at least three different proteins are proteoforms expressed at least partially from the same locus of exons.

40. The method of claim 37, wherein the identifying comprises performing mass spectrometry on the plurality of biomolecules.

41. The method of claim 37, wherein the identifying comprises performing nucleic acid sequencing on the plurality of biomolecules.

42. A method comprising:
(a) contacting a biological sample with a surface to bind a plurality of biomolecules to a peptide coupled to the surface, wherein the peptide is configured to bind to at least five different biomolecules;
(b) releasing the plurality of biomolecules or a portion thereof from the surface; and
(c) identifying at least the plurality of biomolecules or the portion thereof, wherein the plurality of biomolecules or the portion thereof comprises one or more biomolecules in the at least five different biomolecules in the biological sample.

43. The method of claim 42, wherein the identifying in (c) further comprising identifying the plurality of biomolecules, thereby determining interactions between at least a first portion of the five different biomolecules and at least a second portion of the plurality of biomolecules.

44. The method of claim 42, wherein the at least five different biomolecules are proteoforms expressed at least partially from the same locus of exons.

45. The method of claim 42, wherein the identifying comprises performing mass spectrometry on the plurality of biomolecules.

46. The method of claim 42, wherein the identifying comprises performing nucleic acid sequencing on the plurality of biomolecules.

47. A method comprising:
(a) contacting a surface with a first composition, wherein:
i. the surface comprises a peptide coupled thereto, wherein the peptide is configured to specifically bind to at least three different target biomolecules; and
ii. the first composition comprises a plurality of biomolecules, wherein the plurality of biomolecules comprises at least one target biomolecule in the at least three different target biomolecules;
such that:
i. the peptide binds to the at least one target biomolecule in the at least three different target biomolecules; and
ii. the plurality of biomolecules adsorbs on the surface;
(b) contacting the surface with a proteolytic enzyme to release:
i. the at least one target biomolecule;
ii. at least a subset of the plurality of biomolecules adsorbed on the surface; and
iii. at least a portion of the peptide;
thereby producing a second composition;
(c) performing mass spectrometry on the second composition to identify:
i. the at least one target biomolecule;
ii. the at least the subset of the plurality of biomolecules; and
iii. the at least the portion of the peptide;
thereby generating a composition measurement for the first composition; and
(d) removing, from the composition measurement, one or more signals originating from the at least the portion of the peptide, thereby generating a refined composition measurement for the first composition.

48. A method of synthesizing a peptide functionalized surface of the form X-Y-Z, wherein X comprises a surface, Y comprises a linker, and Z comprises a peptide, wherein the synthesizing comprises:
(a) providing the surface;
(b) functionalizing the surface with a linker, then coupling the peptide to the linker; or
(c) coupling the peptide to the linker, then coupling the linker to the surface;
wherein the peptide comprises a binding site configured to bind to at least five different biomolecules.

49. A method of synthesizing a peptide functionalized surface of the form X-Y-Z, wherein X comprises a surface, Y comprises a linker, and Z comprises a peptide, wherein the synthesizing comprises:
(a) providing the surface;
(b) functionalizing the surface with a linker, then coupling the peptide to the linker; or
(c) coupling the peptide to the linker, then coupling the linker to the surface;
wherein the peptide comprises a binding site configured to bind to at least three different proteins.
